SEQUENCE LISTING (207 sequences)

SEQ ID NO: 1 - DNA, 336 bp, Mus sp.
gatattgtgctgacccaatctccactctccctgcctgtcagtcttggagatcaagcctccatctcttgcagatctagtcagagccttgtacacagtaatggaaacacctatttacattggtacctgcagaagccaggccagtctccaaacctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 2 - protein, 112 aa, Mus sp.
DIVLTQSPLSLPVSLGDQASISCRSSQSLVHSNGNTYLHWYLQKPGQSPNLLIYKVSNRFSGVPDRFSGSGSGTDFTLKISRVEAEDLGVYFCSQSTHVPWTFGGGTKLEIK

SEQ ID NO: 3 - DNA, 33 bp, Mus sp.
cagagccttgtacacagtaatggaaacacctat

SEQ ID NO: 4 - DNA, 9 bp, Mus sp.
aaagtttcc

SEQ ID NO: 5 - DNA, 27 bp, Mus sp.
tctcaaagtacacatgttccgtggacg

SEQ ID NO: 6 - protein, 11 aa, Mus sp.
QSLVHSNGNTY

SEQ ID NO: 7 - protein, 3 aa, Mus sp.
KVS

SEQ ID NO: 8 - protein, 9 aa, Mus sp.
SQSTHVPWT

SEQ ID NO: 9 - DNA, 78 bp, Mus sp.
gatattgtgctgacccaatctccactctccctgcctgtcagtcttggagatcaagcctccatctcttgcagatctagt

SEQ ID NO: 10 - DNA, 51 bp, Mus sp.
ttacattggtacctgcagaagccaggccagtctccaaacctcctgatctac

SEQ ID NO: 11 - DNA, 108 bp, Mus sp.
aaccgattttctggggtcccagacaggttcagtggcagtggatcagggacagatttcacactcaagatcagcagagtggaggctgaggatctgggagtttatttctgc

SEQ ID NO: 12 - DNA, 30 bp, Mus sp.
ttcggtggaggcaccaagctggaaatcaaa

SEQ ID NO: 13 - DNA, 358 bp, Mus sp.
gaggtgcagcttgttgagtctggtggaggattggtgcagcctaaaggatcattgaaactctcatgtgccgcctctggtttcgccttcaattcctatgccatgcactgggtctgccaggctccaggaaagggtttggaatgggttgctcgcataagaattaaaagtggtaattatgcaacatcttatgccggttcagtgacagacagattcaccgtctccagagatgattcacaaaacttgttctatctgcaaatgaacaacctgaaaactgaggacacagccatgtattactgtgtgagagagtgggaaggggctatggactactggggtcaaggaacctcagtcaccgtctcctcag

SEQ ID NO: 14 - protein, 119 aa, Mus sp.
EVQLVESGGGLVQPKGSLKLSCAASGFAFNSYAMHWVCQAPGKGLEWVARIRIKSGNYATSYAGSVTDRFTVSRDDSQNLFYLQMNNLKTEDTAMYYCVREWEGAMDYWGQGTSVTVSS

SEQ ID NO: 15 - DNA, 24 bp, Mus sp.
ggtttcgccttcaattcctatgcc

SEQ ID NO: 16 - DNA, 30 bp, Mus sp.
ataagaattaaaagtggtaattatgcaaca

SEQ ID NO: 17 - DNA, 30 bp, Mus sp.
gtgagagagtgggaaggggctatggactac

SEQ ID NO: 18 - protein, 8 aa, Mus sp.
GFAFNSYA

SEQ ID NO: 19 - protein, 10 aa, Mus sp.
IRIKSGNYAT

SEQ ID NO: 20 - protein, 10 aa, Mus sp.
VREWEGAMDY

SEQ ID NO: 21 - DNA, 75 bp, Mus sp.
gaggtgcagcttgttgagtctggtggaggattggtgcagcctaaaggatcattgaaactctcatgtgccgcctct

SEQ ID NO: 22 - DNA, 51 bp, Mus sp.
atgcactgggtctgccaggctccaggaaagggtttggaatgggttgctcgc

SEQ ID NO: 23 - DNA, 114 bp, Mus sp.
tcttatgccggttcagtgacagacagattcaccgtctccagagatgattcacaaaacttgttctatctgcaaatgaacaacctgaaaactgaggacacagccatgtattactgt

SEQ ID NO: 24 - DNA, 34 bp, Mus sp.
tggggtcaaggaacctcagtcaccgtctcctcag

SEQ ID NO: 25 - DNA, 337 bp, Mus sp.
gatgttgtgatgacccagactcccctcactttgtcggttaccattggacagccagcctccatctcttgcacgtcaagtcagagcctcttaaatagtgatggggagacatatttgaattggttgttacagaggccaggccagtctccaaagcgcctaatccatctggtgtctaaactggactctggagtccctgacaggatcactggcagtggatctgggacagatttcacactgaaaatcaacagagtggaggctgaggatttgggaatttattattgctggcaaggtacacattttccgtggacgttcggtggaggcaccaagctggaaatcaaac

SEQ ID NO: 26 - protein, 112 aa, Mus sp.
DVVMTQTPLTLSVTIGQPASISCTSSQSLLNSDGETYLNWLLQRPGQSPKRLIHLVSKLDSGVPDRITGSGSGTDFTLKINRVEAEDLGIYYCWQGTHFPWTFGGGTKLEIK

SEQ ID NO: 27 - DNA, 33 bp, Mus sp.
cagagcctcttaaatagtgatggggagacatat

SEQ ID NO: 28 - DNA, 9 bp, Mus sp.
ctggtgtct

SEQ ID NO: 29 - DNA, 27 bp, Mus sp.
tggcaaggtacacattttccgtggacg

SEQ ID NO: 30 - protein, 11 aa, Mus sp.
QSLLNSDGETY

SEQ ID NO: 31 - protein, 3 aa, Mus sp.
LVS

SEQ ID NO: 32 - protein, 9 aa, Mus sp.
WQGTHFPWT

SEQ ID NO: 33 - DNA, 78 bp, Mus sp.
gatgttgtgatgacccagactcccctcactttgtcggttaccattggacagccagcctccatctcttgcacgtcaagt

SEQ ID NO: 34 - DNA, 51 bp, Mus sp.
ttgaattggttgttacagaggccaggccagtctccaaagcgcctaatccat

SEQ ID NO: 35 - DNA, 108 bp, Mus sp.
aaactggactctggagtccctgacaggatcactggcagtggatctgggacagatttcacactgaaaatcaacagagtggaggctgaggatttgggaatttattattgc

SEQ ID NO: 36 - DNA, 31 bp, Mus sp.
ttcggtggaggcaccaagctggaaatcaaac

SEQ ID NO: 37 - DNA, 349 bp, Mus sp.
caggtccagttgcagcagtctggaactgagctggtaaggcctgggacttcagtgaagatatcctgcaaggcttctggatacgacttcactaacttttggctaggttggataaagcagaggcctggacatggacttgaatggattggagatatttaccctggaggtgataatacttactacaatgagaagttcaagggcaaagtcacgctgactgcagacaaatcctcgaacacagcctatatgcagttcagtagcctgacatctgaggactctgctgtctatttctgttttatgattctctatactttggactactggggtcaaggaacctcagtcaccgtctcctcag

SEQ ID NO: 38 - protein, 116 aa, Mus sp.
QVQLQQSGTELVRPGTSVKISCKASGYDFTNFWLGWIKQRPGHGLEWIGDIYPGGDNTYYNEKFKGKVTLTADKSSNTAYMQFSSLTSEDSAVYFCFMILYTLDYWGQGTSVTVSS

SEQ ID NO: 39 - DNA, 24 bp, Mus sp.
ggatacgacttcactaacttttgg

SEQ ID NO: 40 - DNA, 24 bp, Mus sp.
atttaccctggaggtgataatact

SEQ ID NO: 41 - DNA, 27 bp, Mus sp.
tttatgattctctatactttggactac

SEQ ID NO: 42 - protein, 8 aa, Mus sp.
GYDFTNFW

SEQ ID NO: 43 - protein, 8 aa, Mus sp.
IYPGGDNT

SEQ ID NO: 44 - protein, 9 aa, Mus sp.
FMILYTLDY

SEQ ID NO: 45 - DNA, 75 bp, Mus sp.
caggtccagttgcagcagtctggaactgagctggtaaggcctgggacttcagtgaagatatcctgcaaggcttct

SEQ ID NO: 46 - DNA, 51 bp, Mus sp.
ctaggttggataaagcagaggcctggacatggacttgaatggattggagat

SEQ ID NO: 47 - DNA, 114 bp, Mus sp.
tactacaatgagaagttcaagggcaaagtcacgctgactgcagacaaatcctcgaacacagcctatatgcagttcagtagcctgacatctgaggactctgctgtctatttctgt

SEQ ID NO: 48 - DNA, 34 bp, Mus sp.; identical to SEQ ID NO: 24

SEQ ID NO: 49 - DNA, 337 bp, Mus sp.
gatgttttgatgacccaaactccactctccctgcctgtcagtcttggagatcaagcctccatctcttgcagatctagtcagagcattgtacatagtaatggagacacctatttagaatggtacctgcagaaaccaggccagtctccaaagctcctgatctacaaagtttccaaccgattttctggggtcccagacaggttcagtggcagtggatcagggacagatttcacactcaggatcagcagagtggaggctgaggatctgggagtttattactgctttcaaggttcacattttccgtggacgttcggtggaggcaccaagctggaaatcaaac

SEQ ID NO: 50 - protein, 112 aa, Mus sp.
DVLMTQTPLSLPVSLGDQASISCRSSQSIVHSNGDTYLEWYLQKPGQSPKLLIYKVSNRFSGVPDRFSGSGSGTDFTLRISRVEAEDLGVYYCFQGSHFPWTFGGGTKLEIK

SEQ ID NO: 51 - DNA, 33 bp, Mus sp.
cagagcattgtacatagtaatggagacacctat

SEQ ID NO: 52 - DNA, 9 bp, Mus sp.
aaagtttcc

SEQ ID NO: 53 - DNA, 27 bp, Mus sp.
tttcaaggttcacattttccgtggacg

SEQ ID NO: 54 - protein, 11 aa, Mus sp.
QSIVHSNGDTY

SEQ ID NO: 55 - protein, 3 aa, Mus sp.
KVS

SEQ ID NO: 56 - protein, 9 aa, Mus sp.
FQGSHFPWT

SEQ ID NO: 57 - DNA, 78 bp, Mus sp.
gatgttttgatgacccaaactccactctccctgcctgtcagtcttggagatcaagcctccatctcttgcagatctagt

SEQ ID NO: 58 - DNA, 51 bp, Mus sp.
ttagaatggtacctgcagaaaccaggccagtctccaaagctcctgatctac

SEQ ID NO: 59 - DNA, 108 bp, Mus sp.
aaccgattttctggggtcccagacaggttcagtggcagtggatcagggacagatttcacactcaggatcagcagagtggaggctgaggatctgggagtttattactgc

SEQ ID NO: 60 - DNA, 31 bp, Mus sp.; identical to SEQ ID NO: 36

SEQ ID NO: 61 - DNA, 358 bp, Mus sp.
gatgtgcagctggtggagtctgggggaggcttagtgcagcctggagggtcccggaaactctcctgtacagcctctggattcactttcagtagctttggaatgcactgggttcgtcaggctccagagaaggggctggagtgggtcgcatacattagtagtggcagtagtaaaatctactatgcagacacggtgaagggccgattcaccatctccagagacaatcccaagaacaccctgttcctgcaaatgaccagtctaaggtctgaggacacggccatgtattactgtgcaagagggtggtacgagggggcctggtttgcttactggggccaagggactctggtcactgtctctgcag

SEQ ID NO: 62 - protein, 119 aa, Mus sp.
DVQLVESGGGLVQPGGSRKLSCTASGFTFSSFGMHWVRQAPEKGLEWVAYISSGSSKIYYADTVKGRFTISRDNPKNTLFLQMTSLRSEDTAMYYCARGWYEGAWFAYWGQGTLVTVSA

SEQ ID NO: 63 - DNA, 24 bp, Mus sp.
ggattcactttcagtagctttgga

SEQ ID NO: 64 - DNA, 24 bp, Mus sp.
attagtagtggcagtagtaaaatc

SEQ ID NO: 65 - DNA, 36 bp, Mus sp.
gcaagagggtggtacgagggggcctggtttgcttac

SEQ ID NO: 66 - protein, 8 aa, Mus sp.
GFTFSSFG

SEQ ID NO: 67 - protein, 8 aa, Mus sp.
ISSGSSKI

SEQ ID NO: 68 - protein, 12 aa, Mus sp.
ARGWYEGAWFAY

SEQ ID NO: 69 - DNA, 75 bp, Mus sp.
gatgtgcagctggtggagtctgggggaggcttagtgcagcctggagggtcccggaaactctcctgtacagcctct

SEQ ID NO: 70 - DNA, 51 bp, Mus sp.
atgcactgggttcgtcaggctccagagaaggggctggagtgggtcgcatac

SEQ ID NO: 71 - DNA, 114 bp, Mus sp.
tactatgcagacacggtgaagggccgattcaccatctccagagacaatcccaagaacaccctgttcctgcaaatgaccagtctaaggtctgaggacacggccatgtattactgt

SEQ ID NO: 72 - DNA, 34 bp, Mus sp.
tggggccaagggactctggtcactgtctctgcag

SEQ ID NO: 73 - DNA, 334 bp, Mus sp.
gacattgtgctgacccaatctccagcttctttggctgtgtctctagggcagagggcctccatctcctgcaaggccagccaaagtgttgatcatgatggtgatagttatatgaactggtaccaacagaaaccaggacagccacccaaactcctcatctatgctacatccaatctagaatctgggatcccagccaggtttagtggcagtgggtctgggacagacttcaccctcaacatccatcctgtggaggaggaggatgctgcaacctattactgtcagcagagttatgaggttccgctcacgttcggtgctgggaccaagctggagctgaaac

SEQ ID NO: 74 - protein, 111 aa, Mus sp.
DIVLTQSPASLAVSLGQRASISCKASQSVDHDGDSYMNWYQQKPGQPPKLLIYATSNLESGIPARFSGSGSGTDFTLNIHPVEEEDAATYYCQQSYEVPLTFGAGTKLELK

SEQ ID NO: 75 - DNA, 30 bp, Mus sp.
caaagtgttgatcatgatggtgatagttat

SEQ ID NO: 76 - DNA, 9 bp, Mus sp.
gctacatcc

SEQ ID NO: 77 - DNA, 27 bp, Mus sp.
cagcagagttatgaggttccgctcacg

SEQ ID NO: 78 - protein, 10 aa, Mus sp.
QSVDHDGDSY

SEQ ID NO: 79 - protein, 3 aa, Mus sp.
ATS

SEQ ID NO: 80 - protein, 9 aa, Mus sp.
QQSYEVPLT

SEQ ID NO: 81 - DNA, 78 bp, Mus sp.
gacattgtgctgacccaatctccagcttctttggctgtgtctctagggcagagggcctccatctcctgcaaggccagc

SEQ ID NO: 82 - DNA, 51 bp, Mus sp.
atgaactggtaccaacagaaaccaggacagccacccaaactcctcatctat

SEQ ID NO: 83 - DNA, 108 bp, Mus sp.
aatctagaatctgggatcccagccaggtttagtggcagtgggtctgggacagacttcaccctcaacatccatcctgtggaggaggaggatgctgcaacctattactgt

SEQ ID NO: 84 - DNA, 31 bp, Mus sp.
ttcggtgctgggaccaagctggagctgaaac

SEQ ID NO: 85 - DNA, 343 bp, Mus sp.
gaggtccagctgcaacagtctggacctgagctggtgaagcctggggcttcagtgaagatatcctgcaggacttctggatacacattcactgaatacaccattcactgggtgaagcagagccgtggaaagagccttgagtggattggaggtattaatcctaaccatggtggtactctctacaaccagaagttcaaggtcaaggccacattgactgtagacaagtcctccagcacagcctacatggagctccgcagcctgacatctgaggattctgcagtctattactgtgcaagatttacttacgactactggggccaaggcaccactctcacagtctcctcag

SEQ ID NO: 86 - protein, 114 aa, Mus sp.
EVQLQQSGPELVKPGASVKISCRTSGYTFTEYTIHWVKQSRGKSLEWIGGINPNHGGTLYNQKFKVKATLTVDKSSSTAYMELRSLTSEDSAVYYCARFTYDYWGQGTTLTVSS

SEQ ID NO: 87 - DNA, 24 bp, Mus sp.
ggatacacattcactgaatacacc

SEQ ID NO: 88 - DNA, 24 bp, Mus sp.
attaatcctaaccatggtggtact

SEQ ID NO: 89 - DNA, 21 bp, Mus sp.
gcaagatttacttacgactac

SEQ ID NO: 90 - protein, 8 aa, Mus sp.
GYTFTEYT

SEQ ID NO: 91 - protein, 8 aa, Mus sp.
INPNHGGT

SEQ ID NO: 92 - protein, 7 aa, Mus sp.
ARFTYDY

SEQ ID NO: 93 - DNA, 75 bp, Mus sp.
gaggtccagctgcaacagtctggacctgagctggtgaagcctggggcttcagtgaagatatcctgcaggacttct

SEQ ID NO: 94 - DNA, 51 bp, Mus sp.
attcactgggtgaagcagagccgtggaaagagccttgagtggattggaggt

SEQ ID NO: 95 - DNA, 114 bp, Mus sp.
ctctacaaccagaagttcaaggtcaaggccacattgactgtagacaagtcctccagcacagcctacatggagctccgcagcctgacatctgaggattctgcagtctattactgt

SEQ ID NO: 96 - DNA, 34 bp, Mus sp.
tggggccaaggcaccactctcacagtctcctcag

SEQ ID NO: 97 - DNA, 319 bp, Mus sp.
gaaaatgttctcacccagtctccagcaatcatgtctgcatctccaggggaaaaggtcaccatgacctgcagtgccagctcaagtgtaacttacatgcactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttattactgttttcaggggagtgggtacccgtacacgttcggaggggggaccaagctggaaataaaac

SEQ ID NO: 98 - protein, 106 aa, Mus sp.
ENVLTQSPAIMSASPGEKVTMTCSASSSVTYMHWYQQKSSTSPKLWIYDTSKLASGVPGRFSGSGSGNSYSLTISSMEAEDVATYYCFQGSGYPYTFGGGTKLEIK

SEQ ID NO: 99 - DNA, 21 bp, Mus sp.
gccagctcaagtgtaacttac

SEQ ID NO: 100 - DNA, 9 bp, Mus sp.
gacacatcc

SEQ ID NO: 101 - DNA, 27 bp, Mus sp.
tttcaggggagtgggtacccgtacacg

SEQ ID NO: 102 - protein, 7 aa, Mus sp.
ASSSVTY

SEQ ID NO: 103 - protein, 3 aa, Mus sp.
DTS

SEQ ID NO: 104 - protein, 9 aa, Mus sp.
FQGSGYPYT

SEQ ID NO: 105 - DNA, 72 bp, Mus sp.
gaaaatgttctcacccagtctccagcaatcatgtctgcatctccaggggaaaaggtcaccatgacctgcagt

SEQ ID NO: 106 - DNA, 51 bp, Mus sp.
atgcactggtaccagcagaagtcaagcacctcccccaaactctggatttat

SEQ ID NO: 107 - DNA, 108 bp, Mus sp.
aaactggcttctggagtcccaggtcgcttcagtggcagtgggtctggaaactcttactctctcacgatcagcagcatggaggctgaagatgttgccacttattactgt

SEQ ID NO: 108 - DNA, 31 bp, Mus sp.
ttcggaggggggaccaagctggaaataaaac

SEQ ID NO: 109 - DNA, 349 bp, Mus sp.
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggaaagccaacatatgttgatgacttcaagggacggtttgccttctctttggaaacctctgccaacactgcctatttgcagatcaacaacctcaaaaatgaggacacggctacatatttctgtgaaagtggaggttacgacgaggactactggggccaaggcaccactctcacagtctcctcag

SEQ ID NO: 110 - protein, 116 aa, Mus sp.
QIQLVQSGPELKKPGETVKISCKASGYTFTNYGMNWVKQAPGKGLKWMGWINTYTGKPTYVDDFKGRFAFSLETSANTAYLQINNLKNEDTATYFCESGGYDEDYWGQGTTLTVSS

SEQ ID NO: 111 - DNA, 24 bp, Mus sp.
gggtataccttcacaaactatgga

SEQ ID NO: 112 - DNA, 24 bp, Mus sp.
ataaacacctacactggaaagcca

SEQ ID NO: 113 - DNA, 27 bp, Mus sp.
gaaagtggaggttacgacgaggactac

SEQ ID NO: 114 - protein, 8 aa, Mus sp.
GYTFTNYG

SEQ ID NO: 115 - protein, 8 aa, Mus sp.
INTYTGKP

SEQ ID NO: 116 - protein, 9 aa, Mus sp.
ESGGYDEDY

SEQ ID NO: 117 - DNA, 75 bp, Mus sp.
cagatccagttggtgcagtctggacctgagctgaagaagcctggagagacagtcaagatctcctgcaaggcttct

SEQ ID NO: 118 - DNA, 51 bp, Mus sp.
atgaactgggtgaagcaggctccaggaaagggtttaaagtggatgggctgg

SEQ ID NO: 119 - DNA, 114 bp, Mus sp.
acatatgttgatgacttcaagggacggtttgccttctctttggaaacctctgccaacactgcctatttgcagatcaacaacctcaaaaatgaggacacggctacatatttctgt

SEQ ID NO: 120 - DNA, 34 bp, Mus sp.; identical to SEQ ID NO: 96

SEQ ID NO: 121 - DNA, 337 bp, Mus sp.
gatgttttgatgacccaaactccactctccctgcctgtcagtcttggagatcaagcctccatctcttgcagatctagtcagaacattgtacatagtaatggaaacacctatttagaatggtacctgcagaaatcaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacattttccgtggacgttcggtggaggcaccaagctggaaatcaaac

SEQ ID NO: 122 - protein, 112 aa, Mus sp.
DVLMTQTPLSLPVSLGDQASISCRSSQNIVHSNGNTYLEWYLQKSGQSPKLLIYKVSNRFSGVPDRFSGSGSGTDFTLKISRVEAEDLGVYYCFQGSHFPWTFGGGTKLEIK

SEQ ID NO: 123 - DNA, 33 bp, Mus sp.
cagaacattgtacatagtaatggaaacacctat

SEQ ID NO: 124 - DNA, 9 bp, Mus sp.
aaagtttcc

SEQ ID NO: 125 - DNA, 27 bp, Mus sp.
tttcaaggttcacattttccgtggacg

SEQ ID NO: 126 - protein, 11 aa, Mus sp.
QNIVHSNGNTY

SEQ ID NO: 127 - protein, 3 aa, Mus sp.
KVS

SEQ ID NO: 128 - protein, 9 aa, Mus sp.
FQGSHFPWT

SEQ ID NO: 129 - DNA, 78 bp, Mus sp.; identical to SEQ ID NO: 57

SEQ ID NO: 130 - DNA, 51 bp, Mus sp.
ttagaatggtacctgcagaaatcaggccagtctccaaagctcctgatctac

SEQ ID NO: 131 - DNA, 108 bp, Mus sp.
aaccgattttctggggtcccagacaggttcagtggcagtggatcagggacagatttcacactcaagatcagcagagtggaggctgaggatctgggagtttattactgc

SEQ ID NO: 132 - DNA, 31 bp, Mus sp.; identical to SEQ ID NO: 36

SEQ ID NO: 133 - DNA, 358 bp, Mus sp.
caggtgcagctgaaggagtcaggacctggcctggtggcgccctcacagagcctgtccatcacatgcactgtctcagggttctccttaaccgactatggtataacctggattcgccagcctccaggaaagggtctggagtggctgggagtaatatggggtggtggaagcgcatactataatttagttctcaaatccagactgagcatcagcaaggacaactccaagagtcaagttttcttaaaaatgaacagtctgcaaactgatgacacagccatgtactactgtgccaaacatcggactttgattacgactgctatggactactggggtcaaggaatttcagtcaccgtctcctcag

SEQ ID NO: 134 - protein, 119 aa, Mus sp.
QVQLKESGPGLVAPSQSLSITCTVSGFSLTDYGITWIRQPPGKGLEWLGVIWGGGSAYYNLVLKSRLSISKDNSKSQVFLKMNSLQTDDTAMYYCAKHRTLITTAMDYWGQGISVTVSS

SEQ ID NO: 135 - DNA, 24 bp, Mus sp.
gggttctccttaaccgactatggt

SEQ ID NO: 136 - DNA, 21 bp, Mus sp.
atatggggtggtggaagcgca

SEQ ID NO: 137 - DNA, 39 bp, Mus sp.
gccaaacatcggactttgattacgactgctatggactac

SEQ ID NO: 138 - protein, 8 aa, Mus sp.
GFSLTDYG

SEQ ID NO: 139 - protein, 7 aa, Mus sp.
IWGGGSA

SEQ ID NO: 140 - protein, 13 aa, Mus sp.
AKHRTLITTAMDY

SEQ ID NO: 141 - DNA, 75 bp, Mus sp.
caggtgcagctgaaggagtcaggacctggcctggtggcgccctcacagagcctgtccatcacatgcactgtctca

SEQ ID NO: 142 - DNA, 51 bp, Mus sp.
ataacctggattcgccagcctccaggaaagggtctggagtggctgggagta

SEQ ID NO: 143 - DNA, 114 bp, Mus sp.
tactataatttagttctcaaatccagactgagcatcagcaaggacaactccaagagtcaagttttcttaaaaatgaacagtctgcaaactgatgacacagccatgtactactgt

SEQ ID NO: 144 - DNA, 34 bp, Mus sp.
tggggtcaaggaatttcagtcaccgtctcctcag

SEQ ID NO: 145 - DNA, 337 bp, Mus sp.
gatgttttgatgacccaaactccactctccctgcctgtcagtcttggagatcaagcctccatctcttgcagatctagtcagaacattgtacatagtgatggaaacacctatttagaatggtacctgcagaaaccaggccagtctccaaagctcctgatctacaaattttccaaccgattttctggggtcccagacaggttcagtggcagtggatcagggacagatttcacactcaagatcagcagagtggaggctgaggatctgggagtttattactgctttcaaggttcacatgttcctcccacgttcggtgctgggaccaagctggagctgaaac

SEQ ID NO: 146 - protein, 112 aa, Mus sp.
DVLMTQTPLSLPVSLGDQASISCRSSQNIVHSDGNTYLEWYLQKPGQSPKLLIYKFSNRFSGVPDRFSGSGSGTDFTLKISRVEAEDLGVYYCFQGSHVPPTFGAGTKLELK

SEQ ID NO: 147 - DNA, 33 bp, Mus sp.
cagaacattgtacatagtgatggaaacacctat

SEQ ID NO: 148 - DNA, 9 bp, Mus sp.
aaattttcc

SEQ ID NO: 149 - DNA, 27 bp, Mus sp.
tttcaaggttcacatgttcctcccacg

SEQ ID NO: 150 - protein, 11 aa, Mus sp.
QNIVHSDGNTY

SEQ ID NO: 151 - protein, 3 aa, Mus sp.
KFS

SEQ ID NO: 152 - protein, 9 aa, Mus sp.
FQGSHVPPT

SEQ ID NO: 153 - DNA, 78 bp, Mus sp.; identical to SEQ ID NO: 57

SEQ ID NO: 154 - DNA, 51 bp, Mus sp.; identical to SEQ ID NO: 58

SEQ ID NO: 155 - DNA, 108 bp, Mus sp.; identical to SEQ ID NO: 131

SEQ ID NO: 156 - DNA, 31 bp, Mus sp.; identical to SEQ ID NO: 84

SEQ ID NO: 157 - DNA, 364 bp, Mus sp.
gaagtgaagctggtggagtctgggggaggcttagtgaagtctggagggtccctgaaactctcctgtgcagtctctggattcactttcagtacctatgccatgtcttgggttcgccagattccggagaagaggctggagtgggtcgcaaccattagtaatggtggtagttatatctactattcagacagtgtgaagggtcgattcaccatctccagagacaatgccaagaacaccctgtacctgcaaatgagcagtctgaggtctgaggacacggccatgtattactgtgcacgacatagggagtcctataggtacgactggtttgcttactggggccaagggactctggtcactgtctctgcag

SEQ ID NO: 158 - protein, 121 aa, Mus sp.
EVKLVESGGGLVKSGGSLKLSCAVSGFTFSTYAMSWVRQIPEKRLEWVATISNGGSYIYYSDSVKGRFTISRDNAKNTLYLQMSSLRSEDTAMYYCARHRESYRYDWFAYWGQGTLVTVSA

SEQ ID NO: 159 - DNA, 24 bp, Mus sp.
ggattcactttcagtacctatgcc

SEQ ID NO: 160 - DNA, 24 bp, Mus sp.
attagtaatggtggtagttatatc

SEQ ID NO: 161 - DNA, 42 bp, Mus sp.
gcacgacatagggagtcctataggtacgactggtttgcttac

SEQ ID NO: 162 - protein, 8 aa, Mus sp.
GFTFSTYA

SEQ ID NO: 163 - protein, 8 aa, Mus sp.
ISNGGSYI

SEQ ID NO: 164 - protein, 14 aa, Mus sp.
ARHRESYRYDWFAY

SEQ ID NO: 165 - DNA, 75 bp, Mus sp.
gaagtgaagctggtggagtctgggggaggcttagtgaagtctggagggtccctgaaactctcctgtgcagtctct

SEQ ID NO: 166 - DNA, 51 bp, Mus sp.
atgtcttgggttcgccagattccggagaagaggctggagtgggtcgcaacc

SEQ ID NO: 167 - DNA, 114 bp, Mus sp.
tactattcagacagtgtgaagggtcgattcaccatctccagagacaatgccaagaacaccctgtacctgcaaatgagcagtctgaggtctgaggacacggccatgtattactgt

SEQ ID NO: 168 - DNA, 34 bp, Mus sp.; identical to SEQ ID NO: 72

SEQ ID NO: 169 - protein, 468 aa, Artificial Sequence
  Synthetic Marburg virus Ravn GPe delta muc
MKTIYFLISLILIQSIKTLPVLEIASNSQPQDVDSVCSGTLQKTEDVHLMGFTLSGQKVADSPLEASKRWAFRTGVPPKNVEYTEGEEAKTCYNISVTDPSGKSLLLDPPSNIRDYPKCKTVHHIQGQNPHAQGIALHLWGAFFLYDRVASTTMYRGKVFTEGNIAAMIVNKTVHRMIFSRQGQGYRHMNLTSTNKYWTSSNETQRNDTGCFGILQEYNSTNNQTCPPSLKPPSLPTVTPSIHSTNTQINTAKSGTMRPPIYFRKKRSIFWKEGDIFPFLDGLINTEIDFDPIPNTETIFDESPSFNTSTNEEQHTPPNISLTFSYFPDKNGDTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSKNISEQIDKIRKDEQKEET

SEQ ID NO: 170 - protein, 468 aa, Artificial Sequence
  Synthetic Marburg virus Angola GPe delta muc
MKTTCLLISLILIQGVKTLPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKKPLPLPTAHPEVKLTSTSTDATKLNTTQHLVYFRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGT

SEQ ID NO: 171 - protein, 468 aa, Artificial Sequence
  Synthetic Marburg virus Musoke GPe delta muc
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGT

SEQ ID NO: 172 - protein, 468 aa, Artificial Sequence
  Synthetic Marburg virus Ci67 GPe delta muc
MKTTCLFISLILIQGIKTLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGRVFTEGNIAAMIVNKTVHKMIFSRQGQGYRHMNLTSTNKYWTSNNGTQTNDTGCFGALQEYNSTKNQTCAPSKIPSPLPTARPEIKPTSTPTDATTLNTTQHLVYFRKKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSRNISEQIDQIKKDEQKEGT

SEQ ID NO: 173 - protein, 681 aa, Ravn virus - Ravn, Kenya, 1987
MKTIYFLISLILIQSIKTLPVLEIASNSQPQDVDSVCSGTLQKTEDVHLMGFTLSGQKVADSPLEASKRWAFRTGVPPKNVEYTEGEEAKTCYNISVTDPSGKSLLLDPPSNIRDYPKCKTVHHIQGQNPHAQGIALHLWGAFFLYDRVASTTMYRGKVFTEGNIAAMIVNKTVHRMIFSRQGQGYRHMNLTSTNKYWTSSNETQRNDTGCFGILQEYNSTNNQTCPPSLKPPSLPTVTPSIHSTNTQINTAKSGTMNPSSDDEDLMISGSGSGEQGPHTTLNVVTEQKQSSTILSTPSLHPSTSQHEQNSTNPSRHAVTEHNGTDPTTQPATLLNNTNTTPTYNTLKYNLSTPSPPTRNITNNDTQRELAESEQTNAQLNTTLDPTENPTTAQDTNSTTNIIMTTSDITSKHPTNSSPDSSPTTRPPIYFRKKRSIFWKEGDIFPFLDGLINTEIDFDPIPNTETIFDESPSFNTSTNEEQHTPPNISLTFSYFPDKNGDTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSKNISEQIDKIRKDEQKEETGWGLGGKWWTSDWGVLTNLGILLLLSIAVLIALSCICRIFTKYIG

SEQ ID NO: 174 - protein, 681 aa, Lake Victoria marburgvirus - Angola2005
MKTTCLLISLILIQGVKTLPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKKPLPLPTAHPEVKLTSTSTDATKLNTTDPNSDDEDLTTSGSGSGEQEPYTTSDAATKQGLSSTMPPTPSPQPSTPQQGGNNTNHSQGVVTEPGKTNTTAQPSMPPHNTTTISTNNTSKHNLSTPSVPIQNATNYNTQSTAPENEQTSAPSKTTLLPTENPTTAKSTNSTKSPTTTVPNTTNKYSTSPSPTPNSTAQHLVYFRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 175 - protein, 681 aa, Marburg virus - Musoke, Kenya, 1980
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWGVLTNLGILLLLSIAVLIALSCICRIFTKYIG

SEQ ID NO: 176 - protein, 681 aa, Lake Victoria marburgvirus - Ci67
MKTTCLFISLILIQGIKTLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGRVFTEGNIAAMIVNKTVHKMIFSRQGQGYRHMNLTSTNKYWTSNNGTQTNDTGCFGALQEYNSTKNQTCAPSKIPSPLPTARPEIKPTSTPTDATTLNTTDPNNDDEDLITSGSGSGEQEPYTTSDAVTKQGLSSTMPPTPSPQPSTPQQEGNNTDHSQGTVTEPNKTNTTAQPSMPPHNTTAISTNNTSKNNFSTLSVSLQNTTNYDTQSTATENEQTSAPSKTTLPPTGNLTTAKSTNNTKGPTTTAPNMTNGHLTSPSPTPNPTTQHLVYFRKKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 177 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 178 - protein, 676 aa, Ebola virus - Zaire (1995)
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSPQPPTTKPGPDNSTHNTPVYKLDISEATQVEQHHRRTDNDSTASDTPPATTAAGPLKAENTNTSKGTDLLDPATTTSPQNHSETAGNNNTHHQDTGEESASSGKLGLITNTIAGVAGLITGGRRARREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 179 - protein, 676 aa, Sudan ebolavirus
MEGLSLLQLPRDKFRKSSFFVWVIILFQKAFSMPLGVVTNSTLEVTEIDQLVCKDHLASTDQLKSVGLNLEGSGVSTDIPSATKRWGFRSGVPPQVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAANYTENTSSYYATSYLEYEIENFGAQHSTTLFKINNNTFVLLDRPHTPQFLFQLNDTIQLHQQLSNTTGKLIWTLDANINADIGEWAFWENKKNLSEQLRGEELSFETLSLNETEDDDATSSRTTKGRISDRATRKYSDLVPKDSPGMVSLHVPEGETTLPSQNSTEGRRVDVNTQETITETTATIIGTNGNNMQISTIGTGLSSSQILSSSPTMAPSPETQTSTTYTPKLPVMTTEEPTTPPRNSPGSTTEAPTLTTPENITTAVKTVWAQESTSNGLITSTVTGILGSLGLRKRSRRQVNTRATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 180 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSVTKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQPPTTKTGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 181 - protein, 487 aa, Zaire ebolavirus
MGVTGILQLPRDRFKRTSFFLWVIILFQRTFSIPLGVIHNSTLQVSDVDKLVCRDKLSSTNQLRPVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLEPRFTPQFLLQLNETIYTSGKRSNTTGKLIWKVNPEIDTTIGEWAFWETKKNLTRKIRSEELSFTVVSNTHHQDTGEESASSGKLGLITNTIAGVAGLITGGRRTRREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPD

SEQ ID NO: 182 - protein, 487 aa, Zaire ebolavirus
MGVTGILQLPRDRFKRTSFFLWVIILFQRTFSIPLGVIHNSTLQVSEVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTAVSNTHHQDTGEESASSGKLGLITNTIAGVAGLITGGRRARREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPD

SEQ ID NO: 183 - protein, 487 aa, Sudan ebolavirus
MGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSNITTAVKTVLPQESTSNGLITSTVTGILGSLGLRKRSRRQTNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPN

SEQ ID NO: 184 - protein, 15 aa, Ravn virus - Ravn, Kenya, 1987
LINTEIDFDPIPNTE

SEQ ID NO: 185 - protein, 15 aa, Ravn virus - Ravn, Kenya, 1987
IDFDPIPNTETIFDE

SEQ ID NO: 186 - protein, 15 aa, Ravn virus - Ravn, Kenya, 1987
IDFDPIPNTETIFDE

SEQ ID NO: 187 - protein, 15 aa, Ravn virus - Ravn, Kenya, 1987
IPNTETIFDESPSFN

SEQ ID NO: 188 - protein, 15 aa, Ravn virus - Ravn, Kenya, 1987
PNLDGLINTEIDFDP

SEQ ID NO: 189 - protein, 15 aa, Ravn virus - Ravn, Kenya, 1987
LINTEIDFDPIPNTE

SEQ ID NO: 190 - protein, 15 aa, Ravn virus - Ravn, Kenya, 1987
IDFDPIPNTETIFDE

SEQ ID NO: 191 - protein, 15 aa, Ravn virus - Ravn, Kenya, 1987
LINTEIDFDPIPNTE

SEQ ID NO: 192 - protein, 15 aa, Ravn virus - Ravn, Kenya, 1987
IDFDPIPNTETIFDE

SEQ ID NO: 193 - protein, 15 aa, Ravn virus - Ravn, Kenya, 1987
IPNTETIFDESPSEN

SEQ ID NO: 194 - protein, 15 aa, Ravn virus - Ravn, Kenya, 1987
LINTEIDFDPIPNTE

SEQ ID NO: 195 - protein, 15 aa, Ravn virus - Ravn, Kenya, 1987
IDFDPIPNTETIFDE

SEQ ID NO: 196 - protein, 15 aa, Ravn virus - Ravn, Kenya, 1987
LINTEIDFDPIPNTE

SEQ ID NO: 197 - protein, 15 aa, Ravn virus - Ravn, Kenya, 1987
IDFDPIPNTETIFDE

SEQ ID NO: 198 - protein, 23 aa, Artificial Sequence
  Synthetic Marburg virus Ravn GPe delta muc
DGLINTEIDFDPIPNTETIFDES

SEQ ID NO: 199 - protein, 23 aa, Artificial Sequence
  Synthetic Marburg virus Ci67 GPe delta muc
DGLINAPIDFDPVPNTKTIFDES

SEQ ID NO: 200 - protein, 23 aa, Artificial Sequence
  Synthetic Marburg virus Musoke GPe delta muc
DGLINAPIDFDPVPNTKTIFDES

SEQ ID NO: 201 - protein, 23 aa, Artificial Sequence
  Synthetic Marburg virus Angola GPe delta muc
DGLINAPIDFDPVPNTKTIFDES

SEQ ID NO: 202 - DNA, 337 bp, Mus sp.
gatattgtgctgacccaatctccactctccctgcctgtcagtcttggagatcaagcctccatctcttgcagatctagtcagagccttgtacacagtaatggaaacacctatttacattggtacctgcagaagccaggccagtctccaaacctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaac

SEQ ID NO: 203 - DNA, 358 bp, Mus sp.; identical to SEQ ID NO: 13

SEQ ID NO: 204 - DNA, 334 bp, Mus sp.
gacattgtgctgacccaatctccagcttctttggctgtgtctctagggcagagggcctccatctcctgcaaggccagccaaagtgttgatcatgatggtgatagttatatgaactggtaccaacagaaaccaggacagccacccaaactcctcatctatgctacatccaatctagaatctgggatcccagccaggtttagtggcagtgggtctgggacagacttcaccctcaacatccatcctgtggaggaggaggatgctgcaacctattactgtcagcaaagttatgaggttccgctcacgttcggtgctgggaccaagctggagctgaaac

SEQ ID NO: 205 - DNA, 343 bp, Mus sp.; identical to SEQ ID NO: 85

SEQ ID NO: 206 - DNA, 337 bp, Mus sp.; identical to SEQ ID NO: 25

SEQ ID NO: 207 - DNA, 349 bp, Mus sp.; identical to SEQ ID NO: 37

(19) United States
(12) Patent Application Publication
(10) Pub. No.: US 2017/0065702 A1
(43) Pub. Date: Mar. 9, 2017

(54) METHODS OF MODULATING AN IMMUNE RESPONSE

(71) Applicant: CANGENE CORPORATION, WINNIPEG (CA)

(72) Inventors: Jody BERRY, Winnipeg (CA); Cory NYKIFORUK, Winnipeg (CA)

(21) Appl. No.: 15/120,249

(22) PCT Filed: Feb. 19, 2015
(86) PCT No.: PCT/CA2015/050128

§ 371 Date: Aug. 19, 2016

Related U.S. Application Data
(60) Provisional application No. 61/941,807, filed on Feb. 19, 2014.
(60) Provisional application No. 62/016,419, filed on Jun. 24, 2014.

(51) Int. Cl. A61K 39/12; A61K 39/08; C12N 7/00; C07K 16/10; A61K 39/42

(52) U.S. Cl. CPC A61K 39/12; C07K 16/10; A61K 39/42; C12N 7/00; A61K 39/08; C12N 2760/14234; C12N 2760/14222; C12N 2760/14134; C12N 2760/14122; C07K 2317/34; C07K 2317/76; A61K 2039/505

(57) ABSTRACT

The present invention relates to methods of modulating an immune response in a subject by using a binding protein (e.g., an antibody or an antigen-binding portion of an antibody) to mask or alter an epitope on an antigen that is administered to the subject. Such methods are useful for inducing an immune response (e.g., production of an antibody) in the subject to a non-immunodominant epitope on the antigen. The invention also encompasses an antigen-binding protein complex comprising at least one binding protein bound to at least one immunodominant epitope on an antigen.

This application claims priority to and benefit of U.S. Provisional Patent Application No. 61/941,807, filed on Feb. 19, 2014 and of U.S. Provisional Patent Application No. 62/016,419, filed on Jun. 24, 2014. The contents of both of these applications are herein incorporated by reference in their entirety.

### GOVERNMENT LICENSE RIGHTS

This invention was made in part with government support under Grant No. 1 R01 AI089498-01 awarded by the National Institutes of Health (NIH). The United States government may have certain rights in the invention.

### FIELD OF THE INVENTION

The present invention relates to methods of modulating an immune response in a subject by using a binding protein (e.g., an antibody or an antigen-binding portion of an antibody) to mask an epitope on an antigen that is administered to the subject. The invention also encompasses an antigen-binding protein complex comprising at least one binding protein bound to at least one immunodominant epitope on an antigen.

### BACKGROUND OF THE INVENTION

While the immune system is capable of powerful defenses, some infectious organisms overwhelm a host's immune response. For instance, highly adaptive viruses readily escape cytotoxic T-cell and neutralizing antibody responses and evade the adaptive immune response. Despite more than three decades of research, there is no useful vaccine for the human immunodeficiency virus-1 (HIV-1), in part because of the extraordinary mutation rate arising from an imprecise polymerase and high propensity for recombination between genes and between viral clades. Similarly, other infectious organisms and many cancers do not elicit an effective neutralizing immune response.

Thus, there is a need for new prophylactic and therapeutic approaches that can induce a broad immune response to a specific antigen in a host.

### SUMMARY OF THE INVENTION

The invention encompasses methods and compositions used to induce an immune response to a non-dominant epitope on an immunogenic antigen in a subject. In some embodiments, the invention encompasses a method of inducing an immune response in a subject comprising: (i) administering to the subject an immunogenic composition at least one time wherein the immunogenic composition masks at least one immunodominant epitope on an immunogenic antigen, and (ii) inducing an immune response in the subject to at least one non-immunodominant epitope on the immunogenic antigen. A method may further comprise administering the immunogenic antigen to a subject at least one time.

An immunogenic antigen may comprise at least one immunodominant epitope and at least one non-immunodominant epitope.

An immunogenic composition may comprise an antigen-binding protein complex. A binding protein may be an antibody or an antigen-binding portion thereof and may bind to an immunodominant epitope on the immunogenic antigen. An antibody or antigen-binding portion used in the current invention may be (a) a whole immunoglobulin; (b) an scFv; (c) a Fab fragment; (d) an F(ab′)₂; or (e) a disulfide linked Fv.

An immunogenic antigen suitable in the present invention may be an infectious organism antigen or a tumor cell antigen. An infectious organism antigen may be a viral antigen (e.g, a filovirus antigen) or a bacterial antigen (e.g., a Clostridium difficile antigen). In some embodiments, the filovirus antigen is a Marburg virus antigen or an Ebola virus antigen. For example, the filovirus antigen may be a filovirus glycoprotein. In certain embodiments, the filovirus glycoprotein comprises the GP2 subunit or the GP1 subunit of the Marburg virus glycoprotein. In some embodiments, the Clostridium difficile antigen is C. difficile toxin A or C. difficile toxin B. In some aspects the antigen can be a polypeptide corresponding to a domain or subdomain of the target antigen.

In one aspect, an immune response induced by the methods and compositions described herein is a B-cell response (e.g, the production of an antibody specific to a non-immunodominant epitope on the immunogenic antigen). In some embodiments, the invention further comprises harvesting the antibody specific to a non-immunodominant epitope on the immunogenic antigen from the subject. The harvested antibody may be a neutralizing antibody.

The methods and compositions of the present invention may be used to treat or immunize human and non-human subjects.

In one aspect, the invention encompasses use of an immunogenic composition that masks at least one immunodominant epitope on an immunogenic antigen for inducing an immune response to at least one non-immunodominant epitope on the immunogenic antigen in a subject.

The invention further includes an antigen-binding protein complex comprising at least one binding protein bound to at least one immunodominant epitope on an immunogenic antigen. The binding protein may be an antibody or an antigen-binding portion thereof. For example, the binding protein may be (a) a whole immunoglobulin; (b) an scFv; (c) a Fab fragment; (d) an F(ab′)₂; or (e) a disulfide linked Fv.

The invention also encompasses an antibody (e.g., neutralizing or non-neutralizing but protective) that binds specifically to a non-immunodominant epitope on an immunogenic antigen, wherein the antibody is produced by any of the methods described herein.

These and other embodiments and/or other aspects of the invention will become evident upon reference to the following detailed description of the invention and the attached drawings.

### BRIEF DESCRIPTION OF THE DRAWINGS

FIG. 1 is a schematic showing the domains of the marburgvirus and ebolavirus glycoprotein (GP) and construct design for the antigens described in Example 1. Marburg (MARV) GP and mucin-deleted constructs are shown at the top; Ebola (EBOV) GP and mucin-deleted constructs are shown at the bottom. Dashed lines represent deleted regions. SS, signal sequence; IFL, internal fusion loop; TM, transmembrane. GP ectodomain constructs (GPe) lack the transmembrane (TM) domain and consist of residues 1-637. GP ectodomain mucin-deleted constructs (GPeΔmuc) also lack the mucin-like domain: Δ257-425 for all MARV strains, Δ314-463 for EBOV (Ebola virus), SUDV (Sudan virus), BDBV (Bundibugyo virus) and Δ316-470 for RESTV (Reston virus). As a control for epitope-mapping experiments, an additional MARV GP construct was purified from S2 cells lacking both the GP1 mucin domain (Δ257-425) and the GP2-wing (Δ436-483), termed GPeΔmucΔw.

FIG. 2 is a table showing the results of characterization studies of the CAN30, CAN54 and CAN40 series mAbs. In some of the studies, these antibodies were tested for binding to different strains of MARV and EBOV engineered GPs (Glycoprotein ectodomain, GPe; glycoprotein ectodomain and mucin domain deleted, GPeΔmuc; glycoprotein enzymatically cleaved to resemble the GP core after endosomal cleavage, GPcl). Filovirus species are listed as: M, Musoke; C, Ci67; A, Angola R, Ravn; E, EBOV. Positive binding with the ELISA assay is represented by (+) when mAb concentrations at 10 μg/ml demonstrated OD450>1.0 units above background, and (++) represents stronger binding if values were achieved at 0.5 μg/ml or below. Antibodies considered to have neutralized pseudovirus by a reduction of infectivity <60% of control are marked with a P for partial.

FIG. 3 is a bar graph showing the results of ELISA experiments measuring binding of mAbs CAN30G1 (G1), CAN30G3 (G3), CAN30G4 (G4), CAN30G5 (G5) and CAN30G6 (G6) to GPΔmucΔtm (GPdmuc) of MARV-Ravn (left bar in each set), MARV-Angola (center bar in each set), and MARV-Popp (right bar in each set).

FIG. 4 is a bar graph showing the results of ELISA experiments measuring binding of mAb CAN40G1 (40G1) to various MARV strains. The control mAb was anti-MARV Musoke.

FIG. 5A is a bar graph showing the results of ELISA experiments measuring binding of mAb CAN40G1 (40G1) to various MARV strain and ebolavirus antigens. ELISA plates were coated with each of the ten antigens shown on the x-axis, and CAN40G1 or a control mAb (anti-MARV Musoke) bound at 5 μg/ml. Experiments were performed in triplicate, and standard deviations are displayed.

FIG. 5B shows ELISA binding curves determined by binding CAN40G1 (40G1) or a control mAb (anti-MARV Musoke) to the indicated antigens at a starting concentration of 25 μg/ml, then diluting down by ten to a concentration of 2.5×10⁻⁵ μg/ml. Note that antibody binding affinity for MARV GPeΔmuc, MARV GPcl, and EBOV GPcl is similar causing the curves to overlay.

FIG. 6A is a bar graph showing the results of pseudovirus neutralization assays with mucin-deleted MARV GP after treatment with anti-MARV mAbs (as indicated on the x-axis). Vero cell infectivity of mucin-deleted MARV GP-pseudotyped VSV at an MOI of 0.1 is shown after treatment with 50 μg/ml mAb. Percent infectivity is shown on the y-axis. Grp30polyAb is pooled polyclonal sera from immunized mice. NON is negative control; no antibody added.

FIG. 6B is a bar graph showing the results of pseudovirus neutralization assays with full-length (mucin-containing) MARV GP after treatment with anti-MARV mAbs (as indicated on the x-axis). Vero cell infectivity of mucin-containing MARV GP-pseudotyped VSV at an MOI of 1.0 is shown after treatment with 50 μg/ml mAb. Percent infectivity is shown on the y-axis. Grp30polyAb is pooled polyclonal sera from immunized mice. NON is negative control; no antibody added.

FIG. 7 shows filovirus GP schematics and sequence alignment of mAb epitopes. SS, signal sequence; IFL, internal fusion loop; TM, transmembrane. The furin cleavage site is indicated with an arrowhead where indicated. FIG. 7A shows the Ebola virus GP schematic and construct design. Dashed lines represent deleted regions. GPeΔmuc constructs remove 314-463 from EBOV, BDBV, SUDV and 316-470 from RESTV. FIG. 7B shows the Marburg virus GP schematic. FIG. 7C shows the pepscan defined epitopes for anti-GP2 wing mAbs. This region has four residues unique to strain Ravn. The epitope sequences in this figure are assigned the following SEQ ID NOs: Ravn (SEQ ID NO:198); Ci67 (SEQ ID NO:199); Musoke (SEQ ID NO:200); Angola (SEQ ID NO:201). FIG. 7D shows the results of an experiment where GP2 wing mAb reactivity to Ravn GPeΔmuc wt or E465K was evaluated by ELISA. Positive binding with the ELISA assay is represented by (+) when mAb concentrations at 10 μg/ml demonstrated OD450>1.0 units above background, and (++) represents stronger binding if values were achieved at 0.5 μg/ml or below.

FIG. 8 is a Kaplan Meier plot showing the results of assays examining in vivo protection using mice challenged with a lethal dose of mouse-adapted MARV Ravn. Mice were treated one hour post-exposure with anti-GP antibody; 30G3 (CAN30G3), 30G4 (CAN30G4), 30G5 (CAN30G5), 54G1 (CAN54G1), 54G2 (CAN54G2), 40G1 (CAN40G1), 54G3 (CAN54G3) or PBS alone. Percent survival is shown on the y-axis.

FIG. 9 shows variable (V) gene sequencing results for murine CAN30G5 that include VH and VL sequences from the murine CAN30G5 parental clone.

FIG. 10 shows variable (V) gene sequencing results for murine CAN40G1 that includes VH and VL sequences from the murine CAN40G1 parental clone.

FIG. 11 shows variable (V) gene sequencing results for murine CAN54G2 that includes VH and VL sequences from the murine CAN54G2 parental clone.

FIG. 12 is a diagram of an antigen-antibody complex that may be used for immunization to mask an immunodominant epitope (see, e.g., Example 1).

FIG. 13 shows microcrystals of a complex between Ravn Marburg virus GP and CAN54G1 Fab obtained by microfluidic free interface diffusion.

FIG. 14 is a diagram of the epitopes recognized by anti-C. difficile antibodies that may be used for blocking immunodominant epitopes, thereby eliciting an immune response directed towards the non-immunodominant epitopes.

### DETAILED DESCRIPTION OF THE INVENTION

The invention provides methods of inducing an immune response in a subject to at least one non-immunodominant epitope on an immunogenic antigen by administering to the subject an immunogenic composition that masks at least one immunodominant, non-neutralizing, or ineffective epitope on the immunogenic antigen. The induced immune response may include production of an antibody that binds to the non-immunodominant epitope. The invention encompasses an antibody produced by the induction of an immune response by the methods described herein. In one aspect, the invention provides an antigen-binding protein complex comprising at least one binding protein bound to at least one immunodominant epitope on an immunogenic antigen.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±20% of the indicated range, value, or structure, unless otherwise indicated or apparent from context. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

The invention encompasses a method of inducing or eliciting an immune response in a subject to at least one non-immunodominant epitope on an immunogenic antigen. Immunogenicity is the property enabling a substance (e.g., an antigen) to provoke an immune response, or the degree to which a substance possesses this property. For humoral immunogenicity, immunodominance may be defined as the capacity of certain portions (e.g., epitopes) of the antigen to elicit a larger amount of antibody or more antibodies than other (non-immunodominant) portions of the antigen. B-cell immunodominance may be defined experimentally by characterizing those surfaces of an antigen that elicit the greatest number and/or titer of antibody responses in comparison to those that elicit reduced or absent responses. Focusing B-cell responses from immunodominant regions of an antigen towards immunorecessive regions of the antigen can be determined empirically using the disclosed invention.

In one embodiment, the invention encompasses a method of inducing an immune response in a subject comprising: (i) administering to the subject an immunogenic composition at least one time wherein the immunogenic composition masks at least one immunodominant epitope on an immunogenic antigen, and (ii) inducing an immune response in the subject to at least one non-immunodominant epitope on the immunogenic antigen. The immunogenic composition may be administered to the subject at least two, at least three, at least four, at least five, at least six, at least seven, at least eight, at least nine, or at least ten times, or indefinitely, as long as the desired immune response is induced. In some embodiments, the method may further comprise administering an immunogenic antigen to a subject at least one time. In such embodiments, the immunogenic antigen may be administered before, after, and/or at the same time as the immunogenic composition is administered. In certain embodiments, different immunogenic compositions (antigen-binding protein complexes) may be administered to a subject sequentially or simultaneously.

The invention encompasses an immunogenic antigen. An immunogenic antigen may contain one or more epitopes. For example, an antigen may comprise at least one immunodominant epitope and at least one non-immunodominant epitope. The epitopes may be linear or conformational and may become exposed after binding of a binding protein to the antigen. An immunodominant epitope is defined as an epitope present on an immunogenic antigen wherein the immunization of a subject with the immunogenic antigen elicits an immune response. The immune response generated from the immunization with the immunogenic antigen would result in a high titer of antibodies to the immunodominant epitope. A non-immunodominant epitope or immunorecessive epitope is defined as an epitope present on an immunogenic antigen wherein the immunization of a subject with the immunogenic antigen elicits an immune response with higher titer of antibodies to an immunodominant epitope compared to the immunorecessive or non-immunodominant epitope. In one embodiment, the titer of antibodies generated against the immunodominant epitope would be reduced by immunizing with an antigen-binding protein complex. In another embodiment, the titer of antibodies generated against a non-immunodominant epitope would be increased by immunizing with an antigen-binding protein complex. A non-neutralizing or ineffective epitope may also be masked by the methods and compositions of the invention (for example, identical epitopes on both soluble GP and trimeric GP on the surface of virus to promote immune response to surface GP).

The invention also encompasses a method of exposing a hidden epitope in a conformational antigen comprising complexing a binding protein with the conformational antigen wherein the complexing of the binding protein to the antigen results in a change from the original conformation. The binding protein can be specific to one or more epitopes on the conformational antigen. The change in conformation can be a result of steric hindrance. In certain instances, the binding of a binding protein (e.g., antibody) to one or more epitopes on an antigen may cause the antigen to change conformation thereby exposing epitopes that were hidden in the unbound antigen. Changing the confirmation of the antigen through binding with the binding protein and immunizing a subject with the antigen-antibody binding protein complex can lead to the development of antibodies and an immune response specific to epitopes that would not have been accessible had the subject been immunized with the unbound antigen. Masking of an immunodominant epitope with a binding protein (e.g., Fab) may reduce the creation of neoisotopes as compared to other immunization strategies (e.g., deletion of domains which can introduce new surfaces at the cutting zones as well as altered surfaces around the deleted region) as the antigen remains in a more native state. In the event that a conformation strain is induced on the antigen particle or intact (e.g., viral) target, the opportunity toward development of oligoclonal (or cocktail) antibody approach is also available as passive immunotherapy. In passive immunotherapy, one antibody may be used to induce a conformational change and/or expose an epitope, combined with an additional antibody to target the neoepitope and neutralize, block propagation, or activate classic complementation.

In some embodiments, the instant masking method offers the benefit of retaining the structures of envelope protein or surface molecules in an essentially native state (as monomers, trimers, binding cell surface receptors, membrane fusing moieties, etc.) and avoiding disadvantages of recombinant expression and purification of recombinant surface proteins.

The instant immunomodulating methods provide an advantage over glycan masking, which can lead to reduced immunogenicity in vivo and decrease protein expression levels, suggesting a negative impact on folding efficiency (see, e.g., Bosques et al., 2004, J Am Chem Soc, 126:8421-8425; Rudd et al., 1995, Biochim Biophys Acta, 1248:1-10; Wormald et al., 1999, Structure, 7:R155-160). Glycan masking can be used only at certain protein sites, while the instant methods can be used at a wider variety of sites. In one embodiment, the methods and compositions of the instant invention may be used in combination with glycan masking. For instance, such combination treatments may generate an immune response with a broader neutralization across viral clades.

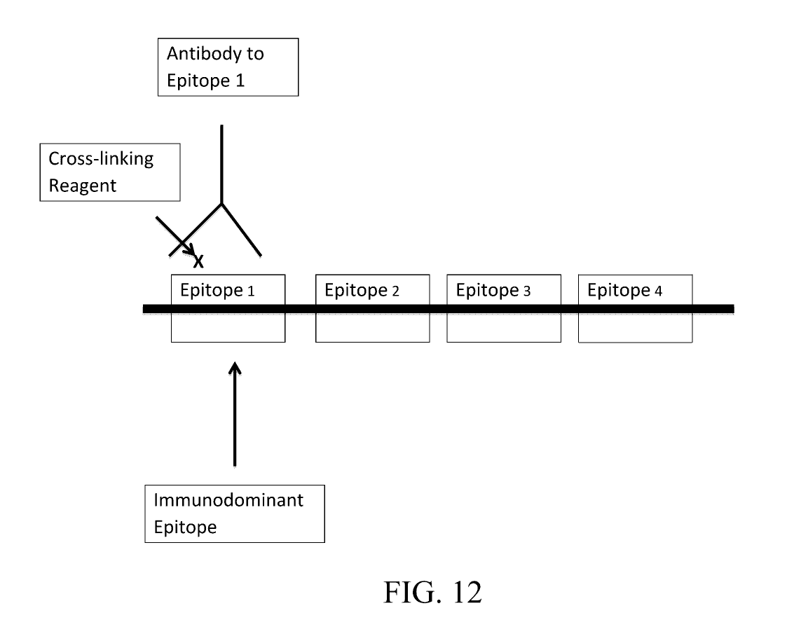

In one aspect, the invention encompasses an antigen-binding protein complex comprising at least one binding protein bound to at least one immunodominant epitope on an immunogenic antigen. Similarly, an immunogenic composition used in the methods of the invention may be an antigen-binding protein complex. A binding protein may mask or alter one or more immunodominant epitopes on the antigen. Binding protein-antigen complexes used in the methods and compositions of the invention can be prepared where the binding protein is directed at one or more epitopes found in the antigen (see FIG. 12 for a diagram where an antibody is shown as an example of a binding protein). The method of preparing an antigen-binding protein complex can also be defined as masking. Binding of the binding protein to a particular epitope or epitopes in an antigen can mask, block, inhibit or reduce the development of an antibody response to that particular epitope when the antigen-binding protein complex is administered to a subject. In certain cases, the epitope is immunodominant when compared with other epitopes in the antigen. Blocking the immune response to the immunodominant epitope (which can include, but is not limited to, the development of antibodies, i.e., IgM and IgG) can allow for the development of an immune response to epitopes other than the immunodominant epitope, herein termed non-immunodominant epitopes. This blockade of the immune response is illustrated in FIG. 12.

A binding protein may be chemically cross-linked with an antigen to form a stable complex. Cross-linking may be accomplished using hetero- or homo-bifunctional cross-linking reagents (Pierce reagents), or other chemical reagents known to generate covalent bonds between molecules (e.g. linkages between intramolcular or intermolecular amino acid residues of two or more polypeptides) also sometimes referred to as bioconjugation. Cross linking reagents known to those in the art include but are not limited to glutaraldehyde, dimethyl adipimaidate, dimethyl adipimidate, dimethyl suberimidate, dimethyl pimelimidate for homo-biofunctional reagents and maleimide, Bis[2-(4-azidosalicylamido)ethyl)] disulfide, succinimidyl 3-(2-pyridyldithio)priopionate], succinimdyl trans-4-(maleimidylmethyl)cyclohexane-1-carboxylate] for hetero-functional reagents. Alternatively chemical oxidation could be used to cross link free sulhydryl groups in close proximity with reagents such as copper (II) chloride, ferric salts, etc. engineered into the binding protein to cross link with antigen.

A binding protein used in the methods of the invention is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, a “polypeptide” or “polypeptide chain” is a single, linear and contiguous arrangement of covalently linked amino acids. Polypeptides can have or form one or more intrachain disulfide bonds. With regard to polypeptides as described herein, reference to amino acid residues corresponding to those specified by SEQ ID NO includes post-translational modifications of such residues. The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence, but is not necessarily at the carboxyl-terminus of the complete polypeptide.

A binding protein used in the methods and compositions of the invention may be an antibody (e.g., neutralizing or non-neutralizing) or an antigen-binding portion thereof. A binding protein may be a whole immunoglobulin, Fab, F(ab′)₂, Fab′, F(ab)′, Fv, single chain Fv (scFv), bivalent scFv (bi-scFv), trivalent scFv (tri-scFv), disulfide linked Fv, Fc, Fd, dAb fragment (e.g., Ward et al., Nature, 341:544-546 (1989)), an isolated CDR, an affibody, a diabody, a triabody, a tetrabody, a linear antibody, a single-chain molecule, a bispecific molecule, a multispecific molecule, or variants, derivatives, combinations and/or mixtures of any of the above. In one embodiment, an immunogenic composition is a Fab-antigen complex. In another embodiment, an immunogenic composition is a F(ab′)₂-antigen complex. As used herein, the term “derivative” refers to a modification of one or more amino acid residues of a peptide by chemical or biological means, either with or without an enzyme, e.g., by glycosylation, alkylation, acylation, ester formation, or amide formation. As used herein, the term “variant” or “variants” refers to a nucleic acid or polypeptide differing from a reference nucleic acid or polypeptide, but retaining essential properties thereof. Generally, variants are overall closely similar, and, in many regions, identical to the reference nucleic acid or polypeptide. For instance, a variant may exhibit at least about 70%, at least about 80%, at least about 90%, at least about 95%, at least about 96%, at least about 97%, at least about 98% or at least about 99% sequence identity compared to the active portion or full length reference nucleic acid or polypeptide.

In some embodiments, a binding protein may be an scFv-scFv dimer, a SMIP, a SCORPION molecule, a BiTE® (Bispecific T-cell Engager) or a diabody. As used herein, the term “SMIP” is used to refer to protein scaffolds as generally disclosed in, for example, in US Patent Application Publication Nos. 2003/0133939, 2003/0118592, and 2005/0136049. A SMIP protein can comprise a polypeptide chain having a binding domain, a hinge region and an immunoglobulin constant region. A “SMIP molecule” should be understood to be a binding protein comprising SMIP scaffolding, e.g., in order from amino to carboxyl-terminus or carboxyl-terminus to amino-terminus, a first binding domain, a hinge region, and an immunoglobulin constant constant region. As used herein, the term “PIMS” is used to refer to protein scaffolds as generally disclosed in, for example, in US Patent Application Publication No. 2009/0148447. A “PIMS molecule” should be understood to be a binding protein comprising PIMS scaffolding, e.g., in order from amino to carboxyl-terminus or carboxyl-terminus to amino-terminus, an immunoglobulin constant region, a hinge region and a first binding domain. As used herein, “SCORPION” is a term used to refer to a multi-specific binding protein scaffold. Multi-specific binding proteins and polypeptides are disclosed, for instance, in PCT Application Publication No. WO 2007/146968, U.S. Patent Application Publication No. 2006/0051844, PCT Application Publication No. WO 2010/040105, PCT Application Publication No. WO 2010/003108, U.S. Pat. No. 7,166,707 and U.S. Pat. No. 8,409,577. A SCORPION polypeptide comprises two binding domains (the domains can be designed to specifically bind the same or different targets), two hinge regions, and an immunoglobulin constant region. SCORPION proteins are homodimeric proteins comprising two identical, disulfide-bonded SCORPION polypeptides. A “SCORPION molecule” should be understood to be a binding protein comprising SCORPION scaffolding, e.g., two binding domains (the domains can be designed to specifically bind the same or different targets), two hinge regions, and an immunoglobulin constant region. BiTE® molecules typically comprise or consist of an anti-antigen scFv linked to an anti-CD3 scFv and typically do not include other sequences such as an immunoglobulin constant region.

A binding protein used in an immunogenic composition of the invention may comprise a binding domain or binding region. As used herein, the term “binding domain” or “binding region” refers to the domain, region, portion, or site of a protein, polypeptide, oligopeptide, or peptide or antibody or binding domain derived from an antibody that possesses the ability to specifically recognize and bind to a target molecule, such as an antigen. Exemplary binding domains include single-chain antibody variable regions (e.g., domain antibodies, sFv, scFv, scFab), receptor ectodomains, and ligands (e.g., cytokines, chemokines). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains that specifically bind a particular target, including Western blot, ELISA, phage display library screening, and BIACORE® interaction analysis.

In addition, it should be understood that the polypeptides comprising the various combinations of the components (e.g., domains or regions) and substituents described herein, are disclosed by the present application to the same extent as if each polypeptide was set forth individually. Thus, selection of particular components of individual polypeptides is within the scope of the present disclosure. As used herein, a binding protein can have a “first binding domain” and, optionally, a “second binding domain.” In certain embodiments, the “first binding domain” binds to an infectious organism antigen or a tumor antigen and the format is an antibody or antibody-like protein or domain. In certain embodiments comprising both the first and second binding domains, the second binding domain is a T-cell binding domain such as a scFv derived from a mouse monoclonal antibody (e.g., CRIS-7) or phage display (e.g., I2C) that binds to a T-cell surface antigen (e.g., CD3).

A whole (or full length) immunoglobulin may be a tetrameric molecule. A tetramer may be composed of two identical pairs of polypeptide chains, each pair having one “light” and one “heavy” chain. The amino-terminal portion of each chain includes a variable region of about 100 to 110 or more amino acids primarily responsible for antigen recognition. The carboxy-terminal portion of each chain defines a constant region primarily responsible for effector function. Human light chains are classified as K and A light chains. Heavy chains are classified as μ, δ, γ, α, or ε, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 or more amino acids. See generally, Fundamental Immunology, Ch. 7 (Paul, W., ed., 2nd ed. Raven Press, N.Y. (1989)). The variable regions of each light/heavy chain pair form the antibody binding site such that an intact immunoglobulin has two binding sites. The terms “light chain variable region” (also referred to as “light chain variable domain” or “VL” or V_{L}) and “heavy chain variable region” (also referred to as “heavy chain variable domain” or “VH” or V_{H}) refer to the variable binding region from an antibody light and heavy chain, respectively. The variable binding regions are made up of discrete, well-defined sub-regions known as “complementarity determining regions” (CDRs) and “framework regions” (FRs). A light chain CDR may be referred to as “LCDR” or “K, CDR.” A heavy chain CDR may be referred to as “HCDR” or “H, CDR.” The heavy chain variable region (or light chain variable region) contains three CDRs and four framework regions (FRs), arranged from amino-terminus to carboxyl-terminus in the following order: FR1, CDR1, FR2, CDR2, FR3, CDR3, FR4. Kabat, E. A., et al. Sequences of Proteins of Immunological Interest, Fifth Edition, U.S. Department of Health and Human Services, NIH Publication No. 91-3242, 1991. Chothia, C. et al., J. Mol. Biol. 196:901-917, 1987. In one embodiment, the FRs are humanized. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 domain of the heavy chain linked to the light chain via an inter-chain disulfide bond.

A binding domain or protein “specifically binds” a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains can be classified as “high affinity” binding domains and “low affinity” binding domains. “High affinity” binding domains refer to those binding domains with a Kₐ of at least about 10⁷ M⁻¹, at least about 10⁸ M⁻¹, at least about 10⁹ M⁻¹, at least about 10¹⁰ M⁻¹, at least about 10¹¹ M⁻¹, at least about 10¹² M⁻¹, or at least about 10¹³ M⁻¹. “Low affinity” binding domains refer to those binding domains with a Kₐ of up to about 10⁷ M⁻¹, up to about 10⁶ M⁻¹, up to about 10⁵ M⁻¹. Alternatively, affinity can be defined as an equilibrium dissociation constant (K_{d}) of a particular binding interaction with units of M (e.g., about 10⁻⁵ M to about 10⁻¹³ M). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent). In some embodiments, the binding protein (e.g., Fab or F(ab′)₂) may possess a submicromolar affinity constant to retain binding and/or masking of the epitope during administration to the subject.

A binding protein or domain can comprise a conservative substitution compared to a known sequence. As used herein, a “conservative substitution” is recognized in the art as a substitution of one amino acid for another amino acid that has similar properties. Exemplary conservative substitutions are well-known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, Mass. (1990), p. 8). In certain embodiments, a conservative substitution includes a leucine to serine substitution.

A binding protein or domain can be derived from an antibody, e.g., a Fab, F(ab′)₂, Fab′, scFv, single domain antibody (sdAb), etc. As used herein, a polypeptide or amino acid sequence “derived from” a designated polypeptide or protein refers to the origin of the polypeptide. In certain embodiments, the polypeptide or amino acid sequence which is derived from a particular sequence (sometimes referred to as the “starting” or “parent” or “parental” sequence) has an amino acid sequence that is essentially identical to the starting sequence or a portion thereof, wherein the portion consists of at least 10-20 amino acids, at least 20-30 amino acids, or at least 30-50 amino acids, or at least 50-150 amino acids, or which is otherwise identifiable to one of ordinary skill in the art as having its origin in the starting sequence.

Polypeptides derived from another polypeptide can have one or more mutations relative to the starting polypeptide, e.g., one or more amino acid residues which have been substituted with another amino acid residue or which has one or more amino acid residue insertions or deletions. The polypeptide can comprise an amino acid sequence which is not naturally occurring. Such variations necessarily have less than 100% sequence identity or similarity with the starting polypeptide. In one embodiment, the variant will have an amino acid sequence from about 60% to less than 100% amino acid sequence identity or similarity with the amino acid sequence of the starting polypeptide. In another embodiment, the variant will have an amino acid sequence from about 75% to less than 100%, from about 80% to less than 100%, from about 85% to less than 100%, from about 90% to less than 100%, from about 95% to less than 100% amino acid sequence identity or similarity with the amino acid sequence of the starting polypeptide.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. The comparison window for nucleic acid sequences can be, for instance, at least about 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least about 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least about 80%, at least about 85%, at least about 90%, at least about 95%, at least about 96%, at least about 97%, at least about 98%, or at least about 99% sequence identity relative to each other.

As used herein, unless otherwise provided, a position of an amino acid residue in a variable region of an immunoglobulin molecule is numbered according to the Kabat numbering convention (Kabat, Sequences of Proteins of Immunological Interest, 5^{th} ed. Bethesda, Md.: Public Health Service, National Institutes of Health (1991)), and a position of an amino acid residue in a constant region of an immunoglobulin molecule is numbered according to EU nomenclature (Ward et al., 1995 Therap. Immunol. 2:77-94).

In some embodiments, a binding protein used in the methods and compositions of the invention is a dimer. As used herein, the term “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis). A “heterodimer” or “heterodimeric protein,” as used herein, refers to a dimer formed from two different polypeptides. A heterodimer does not include an antibody formed from four polypeptides (i.e., two light chains and two heavy chains). A “homodimer” or “homodimeric protein,” as used herein, refers to a dimer formed from two identical polypeptides.

A binding protein may comprise a peptide linker. As used herein, the term “peptide linker” refers to an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of five to about 35 amino acids, for instance, about 15 to about 25 amino acids.

A binding protein or an antibody produced after immunization with an antigen-binding protein complex may be a humanized antibody or antigen-binding portion thereof. As used herein, the term “humanized” refers to a process of making an antibody or immunoglobulin binding proteins and polypeptides derived from a non-human species (e.g., mouse or rat) less immunogenic to humans, while still retaining antigen-binding properties of the original antibody, using genetic engineering techniques. In some embodiments, the binding domain(s) of an antibody or an immunoglobulin binding protein or polypeptide (e.g., light and heavy chain variable regions, Fab, scFv) are humanized. Non-human binding domains can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994: 291-312). If derived from a non-human source, other regions of the antibody or immunoglobulin binding proteins and polypeptides, such as the hinge region and constant region domains, can also be humanized.

In some embodiments, a binding protein used in the methods and compositions of the invention comprises an immunoglobulin constant region. An “immunoglobulin constant region” or “constant region” is a term defined herein to refer to a peptide or polypeptide sequence that corresponds to or is derived from part or all of one or more constant region domains. In certain embodiments, the immunoglobulin constant region corresponds to or is derived from part or all of one or more constant region domains, but not all constant region domains of a source antibody. In certain embodiments, the constant region comprises IgG CH2 and CH3 domains, e.g., IgG1 CH2 and CH3 domains. In certain embodiments, the constant region does not comprise a CH1 domain. In certain embodiments, the constant region domains making up the constant region are human. In some embodiments, the constant region domains of a binding protein used in this invention lack or have minimal effector functions of antibody-dependent cell-mediated cytotoxicity (ADCC) and complement activation and complement-dependent cytotoxicity (CDC), while retaining the ability to bind some F_{c} receptors (such as FcRn, the neonatal Fc receptor) and retaining a relatively long half life in vivo. In other variations, a binding protein of this invention includes constant domains that retain such effector function of one or both of ADCC and CDC. In certain embodiments, a binding domain of this disclosure is fused to a human IgG1 constant region, wherein the IgG1 constant region has one or more of the following amino acids mutated: leucine at position 234 (L234), leucine at position 235 (L235), glycine at position 237 (G237), glutamate at position 318 (E318), lysine at position 320 (K320), lysine at position 322 (K322), or any combination thereof (numbering according to EU). For example, any one or more of these amino acids can be changed to alanine. In a further embodiment, an IgG1 Fc domain has each of L234, L235, G237, E318, K320, and K322 (according to EU numbering) mutated to an alanine (i.e., L234A, L235A, G237A, E318A, K320A, and K322A, respectively), and optionally an N297A mutation as well (i.e., essentially eliminating glycosylation of the CH2 domain).

“Fc region” or “Fc domain” refers to a polypeptide sequence corresponding to or derived from the portion of a source antibody that is responsible for binding to antibody receptors on cells and the C1q component of complement. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, CH2, and CH3 domains. However, more recently the term has been applied to a single chain consisting of CH3, CH2, and at least a portion of the hinge sufficient to form a disulfide-linked dimer with a second such chain. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

“Antibody-dependent cell-mediated cytotoxicity” and “ADCC,” as used herein, refer to a cell-mediated process in which nonspecific cytotoxic cells that express FcγRs (e.g., monocytic cells such as Natural Killer (NK) cells and macrophages) recognize bound antibody (or other protein capable of binding FcγRs) on a target cell and subsequently cause lysis of the target cell. In principle, any effector cell with an activating FcγR can be triggered to mediate ADCC. The primary cells for mediating ADCC are NK cells, which express only FcγRIII, whereas monocytes, depending on their state of activation, localization, or differentiation, can express FcγRI, FcγRII, and FcγRIII. For a review of FcγR expression on hematopoietic cells, see, e.g., Ravetch et al., 1991, Annu. Rev. Immunol., 9:457-92.

The term “having ADCC activity,” as used herein in reference to a polypeptide or protein, means that the polypeptide or protein (for example, one comprising an immunoglobulin hinge region and an immunoglobulin constant region having CH2 and CH3 domains, such as derived from IgG (e.g., IgG1)), is capable of mediating antibody-dependent cell-mediated cytotoxicity (ADCC) through binding of a cytolytic Fc receptor (e.g., FcγRIII) on a cytolytic immune effector cell expressing the Fc receptor (e.g., an NK cell).

“Complement-dependent cytotoxicity” and “CDC,” as used herein, refer to a process in which components in normal serum (“complement”), together with an antibody or other C1q-complement-binding protein bound to a target antigen, exhibit lysis of a target cell expressing the target antigen. Complement consists of a group of serum proteins that act in concert and in an orderly sequence to exert their effect.

The terms “classical complement pathway” and “classical complement system,” as used herein, are synonymous and refer to a particular pathway for the activation of complement. The classical pathway requires antigen-antibody complexes for initiation and involves the activation, in an orderly fashion, of nine major protein components designated C1 through C9. For several steps in the activation process, the product is an enzyme that catalyzes the subsequent step. This cascade provides amplification and activation of large amounts of complement by a relatively small initial signal.

The term “having CDC activity,” as used herein in reference to a polypeptide or protein, means that the polypeptide or protein (for example, one comprising an immunoglobulin hinge region and an immunoglobulin constant region having CH2 and CH3 domains, such as derived from IgG (e.g., IgG1)) is capable of mediating complement-dependent cytotoxicity (CDC) through binding of C1q complement protein and activation of the classical complement system.

As indicated herein, in certain embodiments, the binding proteins used in the methods and compositions of the invention comprise an immunoglobulin constant region (also referred to as a constant region) in a polypeptide chain. By mutations or other alterations, an immunoglobulin constant region further enables relatively easy modulation of dimeric polypeptide effector functions (e.g., ADCC, ADCP, CDC, complement fixation, and binding to Fc receptors), which can either be increased or decreased depending on the disease being treated, as known in the art and described herein. In certain embodiments, an immunoglobulin constant region of one or both of the polypeptide chains of the polypeptide homodimers and heterodimers of the present invention will be capable of mediating one or more of these effector functions In other embodiments, one or more of these effector functions are reduced or absent in an immunoglobulin constant region of one or both of the polypeptide chains of the polypeptide homodimers and heterodimers of the present disclosure, as compared to a corresponding wild-type immunoglobulin constant region. For example, for dimeric binding proteins designed to elicit RTCC, such as, e.g., via the inclusion of a CD3-binding domain, an immunoglobulin constant region preferably has reduced or no effector function relative to a corresponding wild-type immunoglobulin constant region.

An immunoglobulin constant region present in binding proteins of the present invention can comprise or be derived from part or all of: a CH2 domain, a CH3 domain, a CH4 domain, or any combination thereof. For example, an immunoglobulin constant region can comprise a CH2 domain, a CH3 domain, both CH2 and CH3 domains, both CH3 and CH4 domains, two CH3 domains, a CH4 domain, two CH4 domains, and a CH2 domain and part of a CH3 domain.

A CH2 domain that can form an immunoglobulin constant region of a binding protein of the present invention can be a wild type immunoglobulin CH2 domain or an altered immunoglobulin CH2 domain thereof from certain immunoglobulin classes or subclasses (e.g., IgG1, IgG2, IgG3, IgG4, IgA1, IgA2, or IgD) and from various species (including human, mouse, rat, and other mammals).

In certain embodiments, a CH2 domain is a wild type human immunoglobulin CH2 domain, such as wild type CH2 domains of human IgG1, IgG2, IgG3, IgG4, IgA1, IgA2, or IgD, as set forth in SEQ ID NOS:115, 199-201 and 195-197, respectively, of PCT Publication WO2011/090762 (said sequences incorporated by reference herein). In certain embodiments, the CH2 domain is a wild type human IgG1 CH2 domain as set forth in SEQ ID NO:115 of WO2011/090762 (said sequence incorporated by reference herein).

In certain embodiments, a CH2 domain is an altered immunoglobulin CH2 region (e.g., an altered human IgG1 CH2 domain) that comprises an amino acid substitution at the asparagine of position 297 (e.g., asparagine to alanine). Such an amino acid substitution reduces or eliminates glycosylation at this site and abrogates efficient Fc binding to FcγR and C1q. The sequence of an altered human IgG1 CH2 domain with an Asn to Ala substitution at position 297 is set forth in SEQ ID NO:324 of WO2011/090762 said (sequence incorporated by reference herein).

In certain embodiments, a CH2 domain is an altered immunoglobulin CH2 region (e.g., an altered human IgG1 CH2 domain) that comprises at least one substitution or deletion at positions 234 to 238. For example, an immunoglobulin CH2 region can comprise a substitution at position 234, 235, 236, 237 or 238, positions 234 and 235, positions 234 and 236, positions 234 and 237, positions 234 and 238, positions 234-236, positions 234, 235 and 237, positions 234, 236 and 238, positions 234, 235, 237, and 238, positions 236-238, or any other combination of two, three, four, or five amino acids at positions 234-238. In addition or alternatively, an altered CH2 region can comprise one or more (e.g., two, three, four or five) amino acid deletions at positions 234-238, for instance, at one of position 236 or position 237 while the other position is substituted. The above-noted mutation(s) decrease or eliminate the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 has been replaced with one or more alanine residues. In further embodiments, only one of the amino acid residues at positions 234-238 have been deleted while one or more of the remaining amino acids at positions 234-238 can be substituted with another amino acid (e.g., alanine or serine).

In certain other embodiments, a CH2 domain is an altered immunoglobulin CH2 region (e.g., an altered human IgG1 CH2 domain) that comprises one or more amino acid substitutions at positions 253, 310, 318, 320, 322, and 331. For example, an immunoglobulin CH2 region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. The above-noted mutation(s) decrease or eliminate the complement-dependent cytotoxicity (CDC) of a polypeptide heterodimer that comprises the altered CH2 domain.

In certain other embodiments, in addition to the amino acid substitution at position 297, an altered CH2 region (e.g., an altered human IgG1 CH2 domain) can further comprise one or more (e.g., two, three, four, or five) additional substitutions at positions 234-238. For example, an immunoglobulin CH2 region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, an altered CH2 region can comprise one or more (e.g., two, three, four or five) amino acid deletions at positions 234-238, such as at position 236 or position 237. The additional mutation(s) decreases or eliminates the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., alanine or serine).

In certain embodiments, in addition to one or more (e.g., 2, 3, 4, or 5) amino acid substitutions at positions 234-238, a mutated CH2 region (e.g., an altered human IgG1 CH2 domain) in a fusion protein of the present disclosure can contain one or more (e.g., 2, 3, 4, 5, or 6) additional amino acid substitutions (e.g., substituted with alanine) at one or more positions involved in complement fixation (e.g., at positions 1253, H310, E318, K320, K322, or P331). Examples of mutated immunoglobulin CH2 regions include human IgG1, IgG2, IgG4 and mouse IgG2a CH2 regions with alanine substitutions at positions 234, 235, 237 (if present), 318, 320 and 322. An exemplary mutated immunoglobulin CH2 region is mouse IGHG2c CH2 region with alanine substitutions at L234, L235, G237, E318, K320, and K322.

In still further embodiments, in addition to the amino acid substitution at position 297 and the additional deletion(s) or substitution(s) at positions 234-238, an altered CH2 region (e.g., an altered human IgG1 CH2 domain) can further comprise one or more (e.g., two, three, four, five, or six) additional substitutions at positions 253, 310, 318, 320, 322, and 331. For example, an immunoglobulin CH2 region can comprise a (1) substitution at position 297, (2) one or more substitutions or deletions or a combination thereof at positions 234-238, and one or more (e.g., 2, 3, 4, 5, or 6) amino acid substitutions at positions 1253, H310, E318, K320, K322, and P331, such as one, two, three substitutions at positions E318, K320 and K322. The amino acids at the above-noted positions can be substituted by alanine or serine.

In certain embodiments, an immunoglobulin CH2 region polypeptide comprises: (i) an amino acid substitution at the asparagines of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322.

Exemplary altered immunoglobulin CH2 regions with amino acid substitutions at the asparagine of position 297 include: human IgG1 CH2 region with alanine substitutions at L234, L235, G237 and N297 and a deletion at G236 (SEQ ID NO:325 of WO2011/090762, said sequence incorporated by reference herein), human IgG2 CH2 region with alanine substitutions at V234, G236, and N297 (SEQ ID NO:326 of WO2011/090762, said sequence incorporated by reference herein), human IgG4 CH2 region with alanine substitutions at F234, L235, G237 and N297 and a deletion of G236 (SEQ ID NO:322 of WO2011/090762, said sequence incorporated by reference herein), human IgG4 CH2 region with alanine substitutions at F234 and N297 (SEQ ID NO:343 of WO2011/090762, said sequence incorporated by reference herein), human IgG4 CH2 region with alanine substitutions at L235 and N297 (SEQ ID NO:344 of WO2011/090762, said sequence incorporated by reference herein), human IgG4 CH2 region with alanine substitutions at G236 and N297 (SEQ ID NO:345 of WO2011/090762, said sequence incorporated by reference herein), and human IgG4 CH2 region with alanine substitutions at G237 and N297 (SEQ ID NO:346 of WO2011/090762, said sequence incorporated by reference herein).

In certain embodiments, in addition to the amino acid substitutions described above, an altered CH2 region (e.g., an altered human IgG1 CH2 domain) can contain one or more additional amino acid substitutions at one or more positions other than the above-noted positions. Such amino acid substitutions can be conservative or non-conservative amino acid substitutions. For example, in certain embodiments, P233 can be changed to E233 in an altered IgG2 CH2 region (see, e.g., SEQ ID NO:326 of WO2011/090762, said sequence incorporated by reference herein). In addition or alternatively, in certain embodiments, the altered CH2 region can contain one or more amino acid insertions, deletions, or both. The insertion(s), deletion(s) or substitution(s) can be anywhere in an immunoglobulin CH2 region, such as at the N- or C-terminus of a wild type immunoglobulin CH2 region resulting from linking the CH2 region with another region (e.g., a binding domain or an immunoglobulin heterodimerization domain) via a hinge.

In certain embodiments, an altered CH2 region in a polypeptide of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin CH2 region, such as the CH2 region of wild type human IgG1, IgG2, or IgG4, or mouse IgG2a (e.g., IGHG2c).

An altered immunoglobulin CH2 region in a binding protein of the present invention can be derived from a CH2 region of various immunoglobulin isotypes, such as IgG1, IgG2, IgG3, IgG4, IgA1, IgA2, and IgD, from various species (including human, mouse, rat, and other mammals). In certain embodiments, an altered immunoglobulin CH2 region in a fusion protein of the present disclosure can be derived from a CH2 region of human IgG1, IgG2 or IgG4, or mouse IgG2a (e.g., IGHG2c), whose sequences are set forth in SEQ ID NOS:115, 199, 201, and 320 of WO2011/090762 (said sequences incorporated by reference herein).

In certain embodiments, an altered CH2 domain is a human IgG1 CH2 domain with alanine substitutions at positions 235, 318, 320, and 322 (i.e., a human IgG1 CH2 domain with L235A, E318A, K320A and K322A substitutions) (SEQ ID NO:595 of WO2011/090762, said sequence incorporated by reference herein), and optionally an N297 mutation (e.g., to alanine). In certain other embodiments, an altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO:596 of WO2011/090762, said sequence incorporated by reference herein), and optionally an N297 mutation (e.g., to alanine).

In certain embodiments, an altered CH2 domain is an altered human IgG1 CH2 domain with mutations known in the art that enhance immunological activities such as ADCC, ADCP, CDC, complement fixation, Fc receptor binding, or any combination thereof.

The CH3 domain that can form an immunoglobulin constant region of a binding protein of the present invention can be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain is a wild type human immunoglobulin CH3 domain, such as wild type CH3 domains of human IgG1, IgG2, IgG3, IgG4, IgA1, IgA2, IgD, IgE, or IgM as set forth in SEQ ID NOS:116, 208-210, 204-207, and 212, respectively of WO2011/090762 (said sequences incorporated by reference herein). In certain embodiments, the CH3 domain is a wild type human IgG1 CH3 domain as set forth in SEQ ID NO:116 of WO2011/090762 (said sequence incorporated by reference herein). In certain embodiments, a CH3 domain is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain can be a human IgG1 CH3 domain with one or two mutations at positions H433 and N434 (positions are numbered according to EU numbering). The mutations in such positions can be involved in complement fixation. In certain other embodiments, an altered CH3 domain can be a human IgG1 CH3 domain but with one or two amino acid substitutions at position F405 or Y407. The amino acids at such positions are involved in interacting with another CH3 domain. In certain embodiments, an altered CH3 domain can be an altered human IgG1 CH3 domain with its last lysine deleted. The sequence of this altered CH3 domain is set forth in SEQ ID NO:761 of WO2011/090762 (said sequence incorporated by reference herein).

In certain embodiments, binding proteins of the present invention forming a polypeptide heterodimer comprise a CH3 pair that comprises so called “knobs-into-holes” mutations (see, Marvin and Zhu, Acta Pharmacologica Sinica 26:649-58, 2005; Ridgway et al., Protein Engineering 9:617-21, 1966). More specifically, mutations can be introduced into each of the two CH3 domains of each polypeptide chain so that the steric complementarity required for CH3/CH3 association obligates these two CH3 domains to pair with each other. For example, a CH3 domain in one single chain polypeptide of a polypeptide heterodimer can contain a T366W mutation (a “knob” mutation, which substitutes a small amino acid with a larger one), and a CH3 domain in the other single chain polypeptide of the polypeptide heterodimer can contain a Y407A mutation (a “hole” mutation, which substitutes a large amino acid with a smaller one). Other exemplary knobs-into-holes mutations include (1) a T366Y mutation in one CH3 domain and a Y407T in the other CH3 domain, and (2) a T366W mutation in one CH3 domain and T366S, L368A and Y407V mutations in the other CH3 domain.

The CH4 domain that can form an immunoglobulin constant region of a binding protein of the present invention can be a wild type immunoglobulin CH4 domain or an altered immunoglobulin CH4 domain thereof from IgE or IgM molecules. In certain embodiments, the CH4 domain is a wild type human immunoglobulin CH4 domain, such as wild type CH4 domains of human IgE and IgM molecules as set forth in SEQ ID NOS:213 and 214, respectively, of WO2011/090762 (said sequences incorporated by reference herein). In certain embodiments, a CH4 domain is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, an immunoglobulin constant region of a binding protein of the present invention comprises a combination of CH2, CH3 or CH4 domains (i.e., more than one constant region domain selected from CH2, CH3 and CH4). For example, the immunoglobulin constant region can comprise CH2 and CH3 domains or CH3 and CH4 domains. In certain other embodiments, the immunoglobulin constant region can comprise two CH3 domains and no CH2 or CH4 domains (i.e., only two or more CH3). The multiple constant region domains that form an immunoglobulin constant region can be based on or derived from the same immunoglobulin molecule, or the same class or subclass immunoglobulin molecules. In certain embodiments, the immunoglobulin constant region is an IgG CH2CH3 (e.g., IgG1 CH2CH3, IgG2 CH2CH3, and IgG4 CH2CH3) and can be a human (e.g., human IgG1, IgG2, and IgG4) CH2CH3. For example, in certain embodiments, the immunoglobulin constant region comprises (1) wild type human IgG1 CH2 and CH3 domains, (2) human IgG1 CH2 with N297A substitution (i.e., CH2(N297A)) and wild type human IgG1 CH3, or (3) human IgG1 CH2(N297A) and an altered human IgG1 CH3 with the last lysine deleted.

Alternatively, the multiple constant region domains can be based on or derived from different immunoglobulin molecules, or different classes or subclasses immunoglobulin molecules. For example, in certain embodiments, an immunoglobulin constant region comprises both human IgM CH3 domain and human IgG1 CH3 domain. The multiple constant region domains that form an immunoglobulin constant region can be directly linked together or can be linked to each other via one or more (e.g., about 2-10) amino acids.

Exemplary immunoglobulin constant regions are set forth in SEQ ID NOS:305-309, 321, 323, 341, 342, and 762 of WO2011/090762 (said sequences incorporated by reference herein).

In certain embodiments, the immunoglobulin constant regions of both binding proteins of a polypeptide homodimer or heterodimer are identical to each other. In certain other embodiments, the immunoglobulin constant region of one polypeptide chain of a heterodimeric protein is different from the immunoglobulin constant region of the other polypeptide chain of the heterodimer. For example, one immunoglobulin constant region of a heterodimeric protein can contain a CH3 domain with a “knob” mutation, whereas the other immunoglobulin constant region of the heterodimeric protein can contain a CH3 domain with a “hole” mutation.

In some embodiments, a binding protein used in the methods and compositions of the invention is a multispecific molecule that binds to a T-cell receptor, a T-cell receptor complex, or a component of a T-cell receptor complex and may be capable of redirected T-cell cytotoxicity. The binding protein may be a non-neutralizing antibody. “T-cell receptor” (TCR) is a molecule found on the surface of T-cells that, along with CD3, is generally responsible for recognizing antigens bound to major histocompatibility complex (MHC) molecules. It consists of a disulfide-linked heterodimer of the highly variable α and β chains in most T-cells. In other T-cells, an alternative receptor made up of variable γ and δ chains is expressed. Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Abbas and Lichtman, Cellular and Molecular Immunology (5th Ed.), Editor: Saunders, Philadelphia, 2003; Janeway et al., Immunobiology: The Immune System in Health and Disease, 4^{th} Ed., Current Biology Publications, p 148, 149, and 172, 1999). TCR as used in the present disclosure can be from various animal species, including human, mouse, rat, or other mammals.

“TCR complex,” as used herein, refers to a complex formed by the association of CD3 chains with other TCR chains. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRδ chain.

“A component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Redirected T-cell cytotoxicity” and “RTCC,” as used herein, refer to a T-cell-mediated process in which a cytotoxic T-cell is recruited to a target cell using a multispecific protein that is capable of specifically binding both the cytotoxic T-cell and the target cell, and whereby a target-dependent cytotoxic T-cell response is elicited against the target cell.

A binding protein used in the methods and compositions of the invention may bind to any suitable immunogenic antigen. An antigen may be used in its native conformation. An antigen may be an infectious organism antigen or a tumor cell antigen. In some embodiments, an antigen is a viral antigen. A viral antigen may be a recombinant viral subunit, inactivated virus, or live-attenuated virus. Non-limiting example of viral antigens include antigens from filovirus, human immunodeficiency virus, influenza virus A, influenza virus B, and influenza virus C. The filovirus family (Filoviridae) includes two accepted genera, Ebolavirus and Marburgvirus. The Ebolavirus genus includes EBOV (Ebola virus), SUDV (Sudan virus), BDBV (Bundibugyo virus), TAFV (Tai Forest virus) and RESTV (Reston virus). The Marburgvirus genus includes MARV (Marburg virus). All strains of MARV are contemplated for use in this invention (e.g., Ravn, Angola, Musoke, Popp, and Ci67). A filovirus antigen may be a filovirus glycoprotein (GP), which may comprise the GP2 subunit or the GP1 subunit of the Marburg virus glycoprotein. In other embodiments, an antigen is a bacterial antigen. In some embodiments, a filovirus antigen comprises a glycoprotein or glycoprotein precursor amino acid sequence provided in Table 1 (e.g., one of SEQ ID NOs:173-183) or a portion of these sequences. A bacterial antigen may be a Clostridium difficile antigen (e.g., C. difficile toxin A or C. difficile toxin B). The invention also encompasses biological antigens from fungus, plants or other eukaryotic organisms, such as ricin, from which immunotherapies may offer treatment by directing an immune response against immunorecessive epitopes. The invention also encompasses antigens produced synthetically or recombinantly through heterologous expression. In other aspects the antigen can be derived from diseased tissues including but not limited to cancerous tumors. The antigen may be an autoimmune antigen.

The present invention provides methods for inducing an immune response to at least one non-immunodominant epitope on an immunogenic antigen in a subject or patient in need thereof. As used herein, the term “patient in need” refers to a patient at risk of, or suffering from, a disease, disorder or condition that is amenable to treatment or amelioration with a method or composition provided herein. As used herein, the term “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual, or prevent onset of additional associated diseases.

In the methods of the invention, an immunogenic composition (e.g., an antigen-binding protein complex) may be administered to a subject. In therapeutic applications, compositions or medicants are administered to a patient suspected of, or already suffering from such a disorder in an amount sufficient to cure, or at least partially arrest, the symptoms of the disorder and its complications. An amount adequate to accomplish this is referred to as a therapeutically effective dose or amount. As used herein, the term “therapeutically effective amount (or dose)” or “effective amount (or dose)” of a specific binding molecule or compound refers to that amount of the compound sufficient to result in amelioration of one or more symptoms of the disease being treated in a statistically significant manner or a statistically significant improvement in organ function. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or concurrently in separate formulations).

In prophylactic applications, pharmaceutical compositions or medicants are administered to a patient susceptible to, or otherwise at risk of, a particular disorder in an amount sufficient to eliminate or reduce the risk or delay the onset of the disorder. In both prophylactic and therapeutic regimes, agents are usually administered in several dosages until a sufficient response has been achieved. Typically, the response is monitored and repeated dosages are given if the desired response starts to fade. The methods and compositions of the invention may be used in vaccine applications.

An immunogenic composition used herein may comprise a pharmaceutically acceptable carrier, excipient or diluent. As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.” A carrier is said to be a “pharmaceutically acceptable carrier” if its administration can be tolerated by a recipient patient. Sterile phosphate-buffered saline is one example of a pharmaceutically acceptable carrier. Other suitable carriers, diluents or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition comprising an immunogenic composition as described herein may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

A pharmaceutical composition comprising an immunogenic composition may be administered to a subject in a therapeutically effective amount. According to the methods of the present disclosure, an immunogenic composition can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, an antagonist can be administered to a subject in a single bolus delivery, via continuous delivery (e.g., continuous transdermal delivery) over an extended time period, or in a repeated administration protocol (e.g., on an hourly, daily, weekly, or monthly basis).

An immunogenic composition may comprise at least one adjuvant. Adjuvants that may be used to increase the immunogenicity of an antigen, e.g., an immunodominant epitope, include any compound or compounds that act to increase an immune response to peptides or combination of peptides. Non-limiting examples of adjuvants include alum, aluminum phosphate, aluminum hydroxide, MF59 (4.3% w/v squalene, 0.5% w/v polysorbate 80 (Tween 80), 0.5% w/v sorbitan trioleate (Span 85)), CpG-containing nucleic acid, QS21 (saponin adjuvant), MPL (Monophosphoryl Lipid A), 3DMPL (3-O-deacylated MPL), extracts from Aquilla, ISCOMS (see, e.g., Sjolander et al. (1998) J. Leukocyte Biol. 64:713; WO90/03184; WO96/11711; WO 00/48630; WO98/36772; WO00/41720; WO06/134423 and WO07/026190), LT/CT mutants, poly(D,L-lactide-co-glycolide) (PLG) microparticles, Quil A, interleukins, Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

In one aspect, an immune response induced by the methods and compositions described herein is a B-cell response (e.g, the production of an antibody specific to a non-immunodominant epitope on the immunogenic antigen). In some embodiments, the invention further comprises harvesting the antibody specific to a non-immunodominant epitope on the immunogenic antigen from the subject. The harvested antibody may be a neutralizing antibody. A panel of harvested antibodies may be screened for binding (e.g., by ELISA, surface plasmon resonance or Western blot) to the antigen and/or the binding protein used to immunize the subject. Antibodies that bind to the binding protein may be eliminated or subtracted from the panel. As described in Examples 1-3, antibodies CAN54G1, CAN54G2, and CAN54G3 were generated using the methods and compositions of the invention. The amino acid and nucleic acid sequences of the harvested antibody may be obtained and used to generate a humanized antibody by any of the methods described above or generally known in the art. One or more antibodies produced by a subject after immunization by the methods of the invention may be used in a prophylactic (e.g., vaccine) or therapeutic treatment of a disease or disorder caused by an infectious organism comprising the antigen used in the immunization.

In some embodiments, a subject to be immunized or treated by the methods or compositions of the invention is a vertebrate, e.g., a mammal or a non-mammal, including humans, mice, rats, guinea pigs, hamsters, dogs, cats, cows, horses, goats, sheep, pigs, non-human primates, monkeys, apes, gorillas, chimpanzees, rabbits, ducks, geese, chickens, amphibians, reptiles and other animals. The present compositions and methods may be for veterinary use. A subject may be an experimental animal or a transgenic animal. In one aspect, a subject is transgenic and produces human antibodies, e.g., a mouse expressing the human immunoglobulin gene segments (see, e.g., U.S. Pat. Nos. 8,236,311; 7,625,559 and 5,770,429; Lonberg et al., Nature 368(6474): 856-859, 1994; Lonberg, N., Handbook of Experimental Pharmacology 113:49-101, 1994; Lonberg, N. and Huszar, D., Intern. Rev. Immunol., 13: 65-93, 1995; Harding, F. and Lonberg, N., Ann. N.Y. Acad. Sci., 764:536-546, 1995). The invention further encompasses hybridomas generated from animals immunized by the methods and compositions of the invention. Methods of producing hybridomas are generally known in the art and are also described in the Examples.

The present invention provides methods for inducing an immune response to at least one non-immunodominant epitope on an immunogenic antigen in a subject. In some embodiments, a subject treated by the methods or compositions of the invention may be used to generate a hyperimmune. The term hyperimmune, hyperimmune preparation or hyperimmune composition refers to a composition enriched with antibodies specific to one or more particular epitopes. In another embodiment, a subject treated by the methods or compositions of the invention may be used to generate a hyperimmune composition enriched with antibodies to one or more non-immunodominant epitopes and may contain a high titer or concentration of antibodies to one or more non-immunodominant epitopes. In a further embodiment, the antibodies to the non-immunodominant epitopes enriched in the hyperimmune composition are neutralizing.

A hyperimmune preparation of the present disclosure comprises antibodies that may be derived from human or animal plasma after undergoing a series of processing steps. The first step comprises the screening of donor's plasma to identify and collect plasma that demonstrates high titers or elevated serum levels of polyclonal antibodies, particularly high antibody titers to non-immunodominant epitopes. Plasma donors samples having high antibody titers is pooled and fractionated. The primary component of the fractionated pooled plasma is IgG.

Hyperimmune preparations may be prepared by various methods using animal plasma or serum. As used herein, animals can include both humans, non-human primates, as well as other animals, such as horse, sheep, goat, mouse, rabbit, dog, etc. The animal may be artificially immunized with an immunogenic composition via intramuscular, subcutaneous, intraperitoneal or intraocular injection, with or without adjuvant. Samples of serum are collected and tested for reactivity to the non-immunodominant epitope and if required may be boosted with the immunogenic composition one or more times. Once the titer of the animal has reached a plateau in terms of antigen reactivity, larger quantities of antisera may be obtained readily either by periodic bleeding or exsanguinating the non-human animal.

The present invention provides methods for inducing an immune response to at least one non-immunodominant epitope on an immunogenic antigen in a subject. In some embodiments, the immunogenic composition is a vaccine. A vaccine may comprise the antigen-binding protein complex in combination with a pharmaceutically acceptable adjuvant. In one embodiment, the vaccine may be administered to a patient population at risk for developing a disease or at risk of a disease progressing. In one embodiment, for treatment with a vaccine, subjects may be immunized on a schedule that may vary from once a day to once a week, to once a month, to once a year or longer. The schedule may require a booster injection depending on the immune response and physiological condition of the subject. In one embodiment, the antibodies generated on administration of a vaccine to a subject in need may be therapeutic. Treatment with a vaccine is considered therapeutic if at least one symptom of disease in a subject receiving treatment improves or a treatment can delay worsening of a progressive disease in an individual, or prevent onset of additional associated diseases.

The invention will be further clarified by the following examples, which are intended to be purely exemplary of the invention and in no way limiting.

### EXAMPLES

### Example 1

### Production of Immunogenic Composition

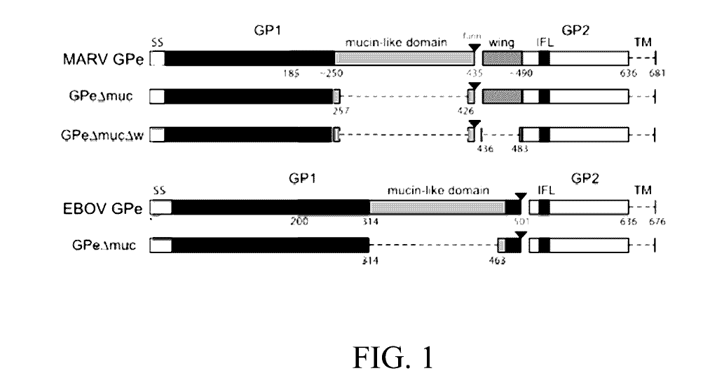

An exemplary immunogenic composition binding to a Marburg virus (Ravn strain) glycoprotein (GP) variant was produced as follows. Ravn GPeΔmuc (SEQ ID NO:169; see FIG. 1 for schematic) was produced in Drosophila S2 cells, purified by streptactin affinity via a C-terminal strep tag, and the trimeric portion isolated on a Superdex 200 sizing column. A CAN30G4 Fab fragment was generated by standard papain digestion of the CAN30G4 IgG antibody (sequences of the CAN30G4 antibody are provided in Table 1) and purified by Mono Q ion-exchange chromatography. (Generation of the CAN30G4 antibody is described in Examples 2 and 3.) Five molar excess Fab was added to the trimeric GPeΔmuc and allowed to bind overnight at 4° C. The complex created was referred to as CAN30G4 Fab-Ravn GPeΔmuc, which was then used to immunize mice (see Example 3). A diagram of the antigen-antibody complex is shown in FIG. 12 (in the absence of a crosslinking reagent for example).

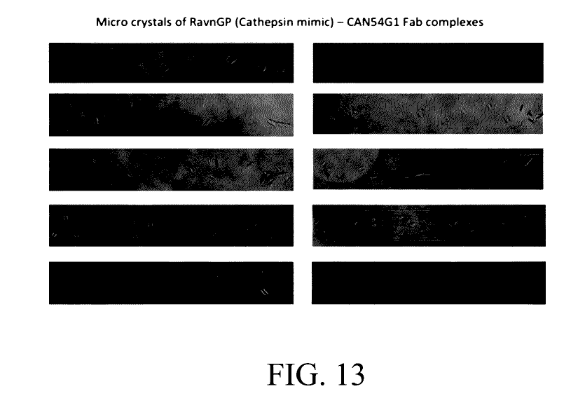

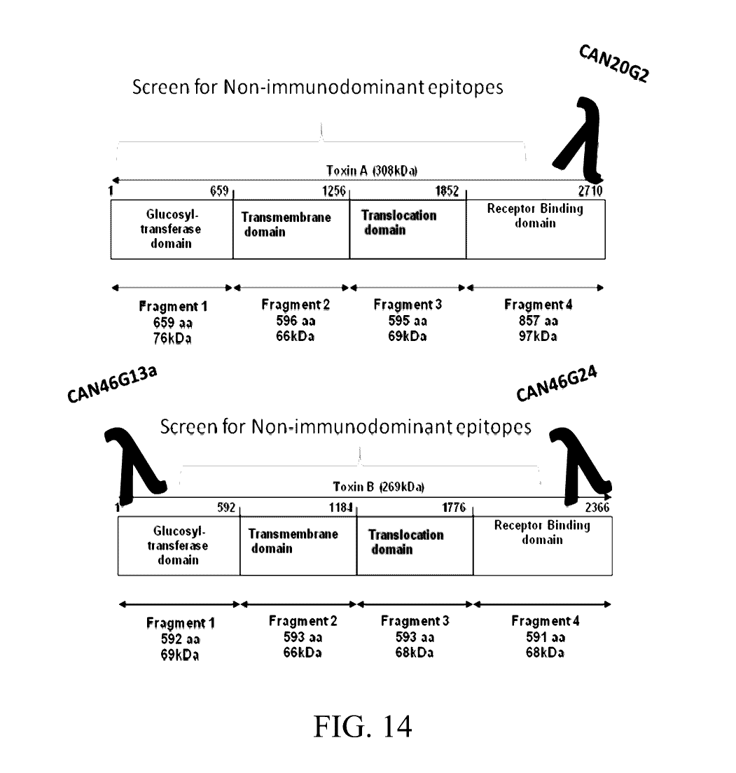

FIG. 13 shows crystals of Marburg virus GP (Ravn) in complex with Fab fragments from mAb CAN54G1. This complex nucleates crystals in multiple conditions.

### Example 2

### Antigen Preparation

Marburgvirus (MARV; Ravn and Angola strains) and ebolavirus glycoprotein (GP) antigens were produced by stable cell line expression in Drosophila S2 and Spodoptera Sf9, or by transient transfection in Gnt−/− HEK293 cells. Proteins were engineered with either a strep or HA tag at the C-terminus to facilitate purification using streptactin (Qiagen) or anti-HA affinity resin (Roche), respectively. GP ectodomain constructs (GPe) lack the transmembrane (TM) domain and consist of residues 1-637. GP ectodomain mucin-deleted constructs (GPeΔmuc) also lack the mucin-like domain: Δ257-425 for all MARV strains, Δ314-463 for EBOV (Ebola virus), SUDV (Sudan virus), BDBV (Bundibugyo virus) and Δ316-470 for RESTV (Reston virus). As a control for epitope-mapping experiments, an additional MARV GP construct was purified from S2 cells lacking both the GP1 mucin domain (Δ257-425) and the GP2-wing (Δ436-483), termed GPeΔmucΔw. GP and GP construct sequences are provided in Table 1. To mimic endosomal cathepsin protease cleavage, cleaved MARV GP (GPcl) was produced by incubation of MARV Ravn strain GPeΔmuc with trypsin a ratio of 1:100 in TBS pH 7.5 at 37° C. for 1 hour. Cleaved EBOV GP was produced by treatment with thermolysin at a ratio of 1:50 overnight at room temperature in TBS pH 7.5 containing 1 mM CaCl₂. GPe proteins were further purified by Superose 6 and all other GP proteins were purified by Superdex 200 size exclusion chromatography. The GP schematic and construct design for the engineered peptides are shown in the diagram in FIG. 1, and a summary of the GP constructs is shown in Table 2.

TABLE 1
Amino acid and nucleotide sequences of antibody domains and antigens

| | | | | SEQ |
|---|---|---|---|---|
| Antibody | Chain, | | | ID |
| Name | Region | Origin | Sequence | NO |
| CAN30G5 | K, | Murine | gatattgtgctgacccaatctccactctccctgcctgtcagtcttgg | 1 |
| | Variable | sequence | agatcaagcctccatctcttgcagatctagtcagagccttgtaca | |
| | region | | cagtaatggaaacacctatttacattggtacctgcagaagccag | |
| | | | gccagtctccaaacctcctgatctacaaagtttccaaccgattttct | |
| | | | ggggtcccagacaggttcagtggcagtggatcagggacagatt | |
| | | | tcacactcaagatcagcagagtggaggctgaggatctgggagt | |
| | | | ttatttctgctctcaaagtacacatgttccgtggacgttcggtggag | |
| | | | gcaccaagctggaaatcaaa | |
| CAN30G5 | K, | Murine | DIVLTQSPLSLPVSLGDQASISCRSSQSLVHSN | 2 |
| | variable | sequence | GNTYLHWYLQKPGQSPNLLIYKVSNRFSGVP | |
| | region | | DRFSGSGSGTDFTLKISRVEAEDLGVYFCSQS | |
| | | | THVPWTFGGGTKLEIK | |
| CAN30G5 | K, | Murine | cagagccttgtacacagtaatggaaacacctat | 3 |
| | CDR1 | sequence | | |
| CAN30G5 | K, | Murine | aaagtttcc | 4 |
| | CDR2 | sequence | | |
| CAN30G5 | K, | Murine | tctcaaagtacacatgttccgtggacg | 5 |
| | CDR3 | sequence | | |
| CAN30G5 | K, | Murine | QSLVHSNGNTY | 6 |
| | CDR1 | sequence | | |
| CAN30G5 | K, | Murine | KVS | 7 |
| | CDR2 | sequence | | |
| CAN30G5 | K, | Murine | SQSTHVPWT | 8 |
| | CDR3 | sequence | | |
| CAN30G5 | K, FR1 | Murine | gatattgtgctgacccaatctccactctccctgcctgtcagtcttgg | 9 |
| | | sequence | agatcaagcctccatctcttgcagatctagt | |
| CAN30G5 | K, FR2 | Murine | ttacattggtacctgcagaagccaggccagtctccaaacctcctg | 10 |
| | | sequence | atctac | |
| CAN30G5 | K, FR3 | Murine | aaccgattttctggggtcccagacaggttcagtggcagtggatca | 11 |
| | | sequence | gggacagatttcacactcaagatcagcagagtggaggctgag | |
| | | | gatctgggagtttatttctgc | |
| CAN30G5 | K, FR4 | Murine | ttcggtggaggcaccaagctggaaatcaaa | 12 |
| | | sequence | | |
| CAN30G5 | H, | Murine | gaggtgcagcttgttgagtctggtggaggattggtgcagcctaaa | 13 |
| | Variable | sequence | ggatcattgaaactctcatgtgccgcctctggtttcgccttcaattcc | |
| | region | | tatgccatgcactgggtctgccaggctccaggaaagggtttgga | |
| | | | atgggttgctcgcataagaattaaaagtggtaattatgcaacatct | |
| | | | tatgccggttcagtgacagacagattcaccgtctccagagatgat | |
| | | | tcacaaaacttgttctatctgcaaatgaacaacctgaaaactgag | |
| | | | gacacagccatgtattactgtgtgagagagtgggaaggggctat | |
| | | | ggactactggggtcaaggaacctcagtcaccgtctcctcag | |
| CAN30G5 | H, | Murine | EVQLVESGGGLVQPKGSLKLSCAASGFAFNS | 14 |
| | variable | sequence | YAMHWVCQAPGKGLEWVARIRIKSGNYATSY | |
| | region | | AGSVTDRFTVSRDDSQNLFYLQMNNLKTEDT | |
| | | | AMYYCVREWEGAMDYWGQGTSVTVSS | |
| CAN30G5 | H, CDR1 | Murine | ggtttcgccttcaattcctatgcc | 15 |
| | | sequence | | |
| CAN30G5 | H, CDR2 | Murine | ataagaattaaaagtggtaattatgcaaca | 16 |
| | | sequence | | |
| CAN30G5 | H, CDR3 | Murine | gtgagagagtgggaaggggctatggactac | 17 |
| | | sequence | | |
| CAN30G5 | H, CDR1 | Murine | GFAFNSYA | 18 |
| | | sequence | | |
| CAN30G5 | H, CDR2 | Murine | IRIKSGNYAT | 19 |
| | | sequence | | |
| CAN30G5 | H, CDR3 | Murine | VREWEGAMDY | 20 |
| | | sequence | | |
| CAN30G5 | H, FR1 | Murine | gaggtgcagcttgttgagtctggtggaggattggtgcagcctaaa | 21 |
| | | sequence | ggatcattgaaactctcatgtgccgcctct | |
| CAN30G5 | H, FR2 | Murine | atgcactgggtctgccaggctccaggaaagggtttggaatgggt | 22 |
| | | sequence | tgctcgc | |
| CAN30G5 | H, FR3 | Murine | tcttatgccggttcagtgacagacagattcaccgtctccagagat | 23 |
| | | sequence | gattcacaaaacttgttctatctgcaaatgaacaacctgaaaact | |
| | | | gaggacacagccatgtattactgt | |
| CAN30G5 | H, FR4 | Murine | tggggtcaaggaacctcagtcaccgtctcctcag | 24 |
| | | sequence | | |
| CAN54G2 | K, | Murine | gatgttgtgatgacccagactcccctcactttgtcggttaccattgg | 25 |
| | Variable | sequence | acagccagcctccatctcttgcacgtcaagtcagagcctcttaaa | |
| | region | | tagtgatggggagacatatttgaattggttgttacagaggccagg | |
| | | | ccagtctccaaagcgcctaatccatctggtgtctaaactggactct | |
| | | | ggagtccctgacaggatcactggcagtggatctgggacagattt | |
| | | | cacactgaaaatcaacagagtggaggctgaggatttgggaattt | |
| | | | attattgctggcaaggtacacattttccgtggacgttcggtggagg | |
| | | | caccaagctggaaatcaaac | |
| CAN54G2 | K, | Murine | DVVMTQTPLTLSVTIGQPASISCTSSQSLLNSD | 26 |
| | variable | sequence | GETYLNWLLQRPGQSPKRLIHLVSKLDSGVPD | |
| | region | | RITGSGSGTDFTLKINRVEAEDLGIYYCWQGT | |
| | | | HFPWTFGGGTKLEIK | |
| CAN54G2 | K, CDR1 | Murine | cagagcctcttaaatagtgatggggagacatat | 27 |
| | | sequence | | |
| CAN54G2 | K, CDR2 | Murine | ctggtgtct | 28 |
| | | sequence | | |
| CAN54G2 | K, CDR3 | Murine | tggcaaggtacacattttccgtggacg | 29 |
| | | sequence | | |
| CAN54G2 | K, CDR1 | Murine | QSLLNSDGETY | 30 |
| | | sequence | | |
| CAN54G2 | K, CDR2 | Murine | LVS | 31 |
| | | sequence | | |
| CAN54G2 | K, CDR3 | Murine | WQGTHFPWT | 32 |
| | | sequence | | |
| CAN54G2 | K, FR1 | Murine | gatgttgtgatgacccagactcccctcactttgtcggttaccattgg | 33 |
| | | sequence | acagccagcctccatctcttgcacgtcaagt | |
| CAN54G2 | K, FR2 | Murine | ttgaattggttgttacagaggccaggccagtctccaaagcgccta | 34 |
| | | sequence | atccat | |
| CAN54G2 | K, FR3 | Murine | aaactggactctggagtccctgacaggatcactggcagtggatc | 35 |
| | | sequence | tgggacagatttcacactgaaaatcaacagagtggaggctgag | |
| | | | gatttgggaatttattattgc | |
| CAN54G2 | K, FR4 | Murine | ttcggtggaggcaccaagctggaaatcaaac | 36 |
| | | sequence | | |
| CAN54G2 | H, | Murine | caggtccagttgcagcagtctggaactgagctggtaaggcctgg | 37 |
| | Variable | sequence | gacttcagtgaagatatcctgcaaggcttctggatacgacttcact | |
| | region | | aacttttggctaggttggataaagcagaggcctggacatggactt | |
| | | | gaatggattggagatatttaccctggaggtgataatacttactaca | |
| | | | atgagaagttcaagggcaaagtcacgctgactgcagacaaat | |
| | | | cctcgaacacagcctatatgcagttcagtagcctgacatctgag | |
| | | | gactctgctgtctatttctgttttatgattctctatactttggactactgg | |
| | | | ggtcaaggaacctcagtcaccgtctcctcag | |
| CAN54G2 | H, | Murine | QVQLQQSGTELVRPGTSVKISCKASGYDFTN | 38 |
| | variable | sequence | FWLGWIKQRPGHGLEWIGDIYPGGDNTYYNE | |
| | region | | KFKGKVTLTADKSSNTAYMQFSSLTSEDSAVY | |
| | | | FCFMILYTLDYWGQGTSVTVSS | |
| CAN54G2 | H, CDR1 | Murine | ggatacgacttcactaacttttgg | 39 |
| | | sequence | | |
| CAN54G2 | H, CDR2 | Murine | atttaccctggaggtgataatact | 40 |
| | | sequence | | |
| CAN54G2 | H, CDR3 | Murine | tttatgattctctatactttggactac | 41 |
| | | sequence | | |
| CAN54G2 | H, CDR1 | Murine | GYDFTNFW | 42 |
| | | sequence | | |
| CAN54G2 | H, CDR2 | Murine | IYPGGDNT | 43 |
| | | sequence | | |
| CAN54G2 | H, CDR3 | Murine | FMILYTLDY | 44 |
| | | sequence | | |
| CAN54G2 | H, FR1 | Murine | caggtccagttgcagcagtctggaactgagctggtaaggcctgg | 45 |
| | | sequence | gacttcagtgaagatatcctgcaaggcttct | |
| CAN54G2 | H, FR2 | Murine | ctaggttggataaagcagaggcctggacatggacttgaatggat | 46 |
| | | sequence | tggagat | |
| CAN54G2 | H, FR3 | Murine | tactacaatgagaagttcaagggcaaagtcacgctgactgcag | 47 |
| | | sequence | acaaatcctcgaacacagcctatatgcagttcagtagcctgaca | |
| | | | tctgaggactctgctgtctatttctgt | |
| CAN54G2 | H, FR4 | Murine | tggggtcaaggaacctcagtcaccgtctcctcag | 48 |
| | | sequence | | |
| CAN30G2 | K, | Murine | gatgttttgatgacccaaactccactctccctgcctgtcagtcttgg | 49 |
| | Variable | sequence | agatcaagcctccatctcttgcagatctagtcagagcattgtacat | |
| | region | | agtaatggagacacctatttagaatggtacctgcagaaaccag | |
| | | | gccagtctccaaagctcctgatctacaaagtttccaaccgattttct | |
| | | | ggggtcccagacaggttcagtggcagtggatcagggacagatt | |
| | | | tcacactcaggatcagcagagtggaggctgaggatctgggagt | |
| | | | ttattactgctttcaaggttcacattttccgtggacgttcggtggagg | |
| | | | caccaagctggaaatcaaac | |
| CAN30G2 | K, | Murine | DVLMTQTPLSLPVSLGDQASISCRSSQSIVHS | 50 |
| | variable | sequence | NGDTYLEWYLQKPGQSPKLLIYKVSNRFSGV | |
| | region | | PDRFSGSGSGTDFTLRISRVEAEDLGVYYCFQ | |
| | | | GSHFPWTFGGGTKLEIK | |
| CAN30G2 | K, CDR1 | Murine | cagagcattgtacatagtaatggagacacctat | 51 |
| | | sequence | | |
| CAN30G2 | K, CDR2 | Murine | aaagtttcc | 52 |
| | | sequence | | |
| CAN30G2 | K, CDR3 | Murine | tttcaaggttcacattttccgtggacg | 53 |
| | | sequence | | |
| CAN30G2 | K, CDR1 | Murine | QSIVHSNGDTY | 54 |
| | | sequence | | |
| CAN30G2 | K, CDR2 | Murine | KVS | 55 |
| | | sequence | | |
| CAN30G2 | K, CDR3 | Murine | FQGSHFPWT | 56 |
| | | sequence | | |
| CAN30G2 | K, FR1 | Murine | gatgttttgatgacccaaactccactctccctgcctgtcagtcttgg | 57 |
| | | sequence | agatcaagcctccatctcttgcagatctagt | |
| CAN30G2 | K, FR2 | Murine | ttagaatggtacctgcagaaaccaggccagtctccaaagctcct | 58 |
| | | sequence | gatctac | |
| CAN30G2 | K, FR3 | Murine | aaccgattttctggggtcccagacaggttcagtggcagtggatca | 59 |
| | | sequence | gggacagatttcacactcaggatcagcagagtggaggctgag | |
| | | | gatctgggagtttattactgc | |
| CAN30G2 | K, FR4 | Murine | ttcggtggaggcaccaagctggaaatcaaac | 60 |
| | | sequence | | |
| CAN30G2 | H, | Murine | gatgtgcagctggtggagtctgggggaggcttagtgcagcctgg | 61 |
| | Variable | sequence | agggtcccggaaactctcctgtacagcctctggattcactttcagt | |
| | region | | agctttggaatgcactgggttcgtcaggctccagagaaggggct | |
| | | | ggagtgggtcgcatacattagtagtggcagtagtaaaatctacta | |
| | | | tgcagacacggtgaagggccgattcaccatctccagagacaat | |
| | | | cccaagaacaccctgttcctgcaaatgaccagtctaaggtctga | |
| | | | ggacacggccatgtattactgtgcaagagggtggtacgagggg | |
| | | | gcctggtttgcttactggggccaagggactctggtcactgtctctg | |
| | | | cag | |
| CAN30G2 | H, | Murine | DVQLVESGGGLVQPGGSRKLSCTASGFTFSS | 62 |
| | variable | sequence | FGMHWVRQAPEKGLEWVAYISSGSSKIYYAD | |
| | region | | TVKGRFTISRDNPKNTLFLQMTSLRSEDTAMY | |
| | | | YCARGWYEGAWFAYWGQGTLVTVSA | |
| CAN30G2 | H, CDR1 | Murine | ggattcactttcagtagctttgga | 63 |
| | | sequence | | |
| CAN30G2 | H, CDR2 | Murine | attagtagtggcagtagtaaaatc | 64 |
| | | sequence | | |
| CAN30G2 | H, CDR3 | Murine | gcaagagggtggtacgagggggcctggtttgcttac | 65 |
| | | sequence | | |
| CAN30G2 | H, CDR1 | Murine | GFTFSSFG | 66 |
| | | sequence | | |
| CAN30G2 | H, CDR2 | Murine | ISSGSSKI | 67 |
| | | sequence | | |
| CAN30G2 | H, CDR3 | Murine | ARGWYEGAWFAY | 68 |
| | | sequence | | |
| CAN30G2 | H, FR1 | Murine | gatgtgcagctggtggagtctgggggaggcttagtgcagcctgg | 69 |
| | | sequence | agggtcccggaaactctcctgtacagcctct | |
| CAN30G2 | H, FR2 | Murine | atgcactgggttcgtcaggctccagagaaggggctggagtggg | 70 |
| | | sequence | tcgcatac | |
| CAN30G2 | H, FR3 | Murine | tactatgcagacacggtgaagggccgattcaccatctccagag | 71 |
| | | sequence | acaatcccaagaacaccctgttcctgcaaatgaccagtctaag | |
| | | | gtctgaggacacggccatgtattactgt | |
| CAN30G2 | H, FR4 | Murine | tggggccaagggactctggtcactgtctctgcag | 72 |
| | | sequence | | |
| CAN40G1 | K, | Murine | gacattgtgctgacccaatctccagcttctttggctgtgtctctagg | 73 |
| | Variable | sequence | gcagagggcctccatctcctgcaaggccagccaaagtgttgat | |
| | region | | catgatggtgatagttatatgaactggtaccaacagaaaccagg | |
| | | | acagccacccaaactcctcatctatgctacatccaatctagaatc | |
| | | | tgggatcccagccaggtttagtggcagtgggtctgggacagact | |
| | | | tcaccctcaacatccatcctgtggaggaggaggatgctgcaac | |
| | | | ctattactgtcagcagagttatgaggttccgctcacgttaggtgctg | |
| | | | ggaccaagctggagctgaaac | |
| CAN40G1 | K, | Murine | DIVLTQSPASLAVSLGQRASISCKASQSVDHD | 74 |
| | variable | sequence | GDSYMNWYQQKPGQPPKLLIYATSNLESGIP | |
| | region | | ARFSGSGSGTDFTLNIHPVEEEDAATYYCQQ | |
| | | | SYEVPLTFGAGTKLELK | |
| CAN40G1 | K, CDR1 | Murine | caaagtgttgatcatgatggtgatagttat | 75 |
| | | sequence | | |
| CAN40G1 | K, CDR2 | Murine | gctacatcc | 76 |
| | | sequence | | |
| CAN40G1 | K, CDR3 | Murine | cagcagagttatgaggttccgctcacg | 77 |
| | | sequence | | |
| CAN40G1 | K, CDR1 | Murine | QSVDHDGDSY | 78 |
| | | sequence | | |
| CAN40G1 | K, CDR2 | Murine | ATS | 79 |
| | | sequence | | |
| CAN40G1 | K, CDR3 | Murine | QQSYEVPLT | 80 |
| | | sequence | | |
| CAN40G1 | K, FR1 | Murine | gacattgtgctgacccaatctccagcttctttggctgtgtctctagg | 81 |
| | | sequence | gcagagggcctccatctcctgcaaggccagc | |
| CAN40G1 | K, FR2 | Murine | atgaactggtaccaacagaaaccaggacagccacccaaact | 82 |
| | | sequence | cctcatctat | |
| CAN40G1 | K, FR3 | Murine | aatctagaatctgggatcccagccaggtttagtggcagtgggtct | 83 |
| | | sequence | gggacagacttcaccdcaacatccatcctgtggaggaggagg | |
| | | | atgctgcaacctattactgt | |
| CAN40G1 | K, FR4 | Murine | ttcggtgctgggaccaagctggagctgaaac | 84 |
| | | sequence | | |
| CAN40G1 | H, | Murine | gaggtccagctgcaacagtctggacctgagctggtgaagcctg | 85 |
| | Variable | sequence | gggcttcagtgaagatatcctgcaggacttctggatacacattca | |
| | region | | ctgaatacaccattcactgggtgaagcagagccgtggaaaga | |
| | | | gccttgagtggattggaggtattaatcctaaccatggtggtactct | |
| | | | ctacaaccagaagttcaaggtcaaggccacattgactgtagac | |
| | | | aagtcctccagcacagcctacatggagctccgcagcctgacat | |
| | | | ctgaggattctgcagtctattactgtgcaagatttacttacgactact | |
| | | | ggggccaaggcaccactctcacagtctcctcag | |
| CAN40G1 | H, | Murine | EVQLQQSGPELVKPGASVKISCRTSGYTFTEY | 86 |
| | variable | sequence | TIHWVKQSRGKSLEWIGGINPNHGGTLYNQK | |
| | region | | FKVKATLTVDKSSSTAYMELRSLTSEDSAVYY | |
| | | | CARFTYDYWGQGTTLTVSS | |
| CAN40G1 | H, CDR1 | Murine | ggatacacattcactgaatacacc | 87 |
| | | sequence | | |
| CAN40G1 | H, CDR2 | Murine | attaatcctaaccatggtggtact | 88 |
| | | sequence | | |
| CAN40G1 | H, CDR3 | Murine | gcaagatttacttacgactac | 89 |
| | | sequence | | |
| CAN40G1 | H, CDR1 | Murine | GYTFTEYT | 90 |
| | | sequence | | |
| CAN40G1 | H, CDR2 | Murine | INPNHGGT | 91 |
| | | sequence | | |
| CAN40G1 | H, CDR3 | Murine | ARFTYDY | 92 |
| | | sequence | | |
| CAN40G1 | H, FR1 | Murine | gaggtccagctgcaacagtctggacctgagctggtgaagcctg | 93 |
| | | sequence | gggcttcagtgaagatatcctgcaggacttct | |
| CAN40G1 | H, FR2 | Murine | attcactgggtgaagcagagccgtggaaagagccttgagtgga | 94 |
| | | sequence | ttggaggt | |
| CAN40G1 | H, FR3 | Murine | ctctacaaccagaagttcaaggtcaaggccacattgactgtaga | 95 |
| | | sequence | caagtcctccagcacagcctacatggagctccgcagcctgaca | |
| | | | tctgaggattctgcagtctattactgt | |
| CAN40G1 | H, FR4 | Murine | tggggccaaggcaccactctcacagtctcctcag | 96 |
| | | sequence | | |
| CAN30G1 | K, | Murine | gaaaatgttctcacccagtctccagcaatcatgtctgcatctccag | 97 |
| | Variable | sequence | gggaaaaggtcaccatgacctgcagtgccagctcaagtgtaac | |
| | region | | ttacatgcactggtaccagcagaagtcaagcacctcccccaaa | |
| | | | ctctggatttatgacacatccaaactggcttctggagtccaggtc | |
| | | | gcttcagtggcagtgggtctggaaactcttactctctcacgatcag | |
| | | | cagcatggaggctgaagatgttgccacttattactgttttcagggg | |
| | | | agtgggtacccgtacacgttcggaggggggaccaagctggaa | |
| | | | ataaaac | |
| CAN30G1 | K, | Murine | ENVLTQSPAIMSASPGEKVTMTCSASSSVTY | 98 |
| | variable | sequence | MHWYQQKSSTSPKLWIYDTSKLASGVPGRFS | |
| | region | | GSGSGNSYSLTISSMEAEDVATYYCFQGSGY | |
| | | | PYTFGGGTKLEIK | |
| CAN30G1 | K, CDR1 | Murine | gccagctcaagtgtaacttac | 99 |
| | | sequence | | |
| CAN30G1 | K, CDR2 | Murine | gacacatcc | 100 |
| | | sequence | | |
| CAN30G1 | K, CDR3 | Murine | tttcaggggagtgggtacccgtacacg | 101 |
| | | sequence | | |
| CAN30G1 | K, CDR1 | Murine | ASSSVTY | 102 |
| | | sequence | | |
| CAN30G1 | K, CDR2 | Murine | DTS | 103 |
| | | sequence | | |
| CAN30G1 | K, CDR3 | Murine | FQGSGYPYT | 104 |
| | | sequence | | |
| CAN30G1 | K, FR1 | Murine | gaaaatgttctcacccagtctccagcaatcatgtctgcatctccag | 105 |
| | | sequence | gggaaaaggtcaccatgacctgcagt | |
| CAN30G1 | K, FR2 | Murine | atgcactggtaccagcagaagtcaagcacctcccccaaactct | 106 |
| | | sequence | ggatttat | |
| CAN30G1 | K, FR3 | Murine | aaactggcttctggagtccaggtcgcttcagtggcagtgggtct | 107 |
| | | sequence | ggaaactcttactctctcacgatcagcagcatggaggctgaaga | |
| | | | tgttgccacttattactgt | |
| CAN30G1 | K, FR4 | Murine | ttcggaggggggaccaagctggaaataaaac | 108 |
| | | sequence | | |
| CAN30G1 | H, | Murine | cagatccagttggtgcagtctggacctgagctgaagaagcctgg | 109 |
| | Variable | sequence | agagacagtcaagatctcctgcaaggcttctgggtataccttcac | |
| | region | | aaactatggaatgaactgggtgaagcaggctccaggaaaggg | |
| | | | tttaaagtggatgggctggataaacacctacactggaaagcca | |
| | | | acatatgttgatgacttcaagggacggtttgccttctctttggaaac | |
| | | | ctctgccaacactgcctatttgcagatcaacaacctcaaaaatg | |
| | | | aggacacggctacatatttctgtgaaagtggaggttacgacgag | |
| | | | gactactggggccaaggcaccactctcacagtctcctcag | |
| CAN30G1 | H, | Murine | QIQLVQSGPELKKPGETVKISCKASGYTFTNY | 110 |
| | variable | sequence | GMNWVKQAPGKGLKWMGWINTYTGKPTYVD | |
| | region | | DFKGRFAFSLETSANTAYLQINNLKNEDTATY | |
| | | | FCESGGYDEDYWGQGTTLTVSS | |
| CAN30G1 | H, CDR1 | Murine | gggtataccttcacaaactatgga | 111 |
| | | sequence | | |
| CAN30G1 | H, CDR2 | Murine | ataaacacctacactggaaagcca | 112 |
| | | sequence | | |
| CAN30G1 | H, CDR3 | Murine | gaaagtggaggttacgacgaggactac | 113 |
| | | sequence | | |
| CAN30G1 | H, CDR1 | Murine | GYTFTNYG | 114 |
| | | sequence | | |
| CAN30G1 | H, CDR2 | Murine | INTYTGKP | 115 |
| | | sequence | | |
| CAN30G1 | H, CDR3 | Murine | ESGGYDEDY | 116 |
| | | sequence | | |
| CAN30G1 | H, FR1 | Murine | cagatccagttggtgcagtctggacctgagctgaagaagcctgg | 117 |
| | | sequence | agagacagtcaagatctcctgcaaggcttct | |
| CAN30G1 | H, FR2 | Murine | atgaactgggtgaagcaggctccaggaaagggtttaaagtgga | 118 |
| | | sequence | tgggctgg | |
| CAN30G1 | H, FR3 | Murine | acatatgttgatgacttcaagggacggtttgccttctctttggaaac | 119 |
| | | sequence | ctctgccaacactgcctatttgcagatcaacaacctcaaaaatg | |
| | | | aggacacggctacatatttctgt | |
| CAN30G1 | H, FR4 | Murine | tggggccaaggcaccactctcacagtctcctcag | 120 |
| | | sequence | | |
| CAN30G3 | K, | Murine | gatgttttgatgacccaaactccactctccctgcctgtcagtcttgg | 121 |
| | Variable | sequence | agatcaagcctccatctcttgcagatctagtcagaacattgtacat | |
| | region | | agtaatggaaacacctatttagaatggtacctgcagaaatcagg | |
| | | | ccagtctccaaagctcctgatctacaaagtttccaaccgattttctg | |
| | | | gggtcccagacaggttcagtggcagtggatcagggacagattt | |
| | | | cacactcaagatcagcagagtggaggctgaggatctgggagtt | |
| | | | tattactgctttcaaggttcacattttccgtggacgttcggtggaggc | |
| | | | accaagctggaaatcaaac | |
| CAN30G3 | K, | Murine | DVLMTQTPLSLPVSLGDQASISCRSSQNIVHS | 122 |
| | variable | sequence | NGNTYLEWYLQKSGQSPKLLIYKVSNRFSGV | |
| | region | | PDRFSGSGSGTDFTLKISRVEAEDLGVYYCFQ | |
| | | | GSHFPWTFGGGTKLEIK | |
| CAN30G3 | K, CDR1 | Murine | cagaacattgtacatagtaatggaaacacctat | 123 |
| | | sequence | | |
| CAN30G3 | K, CDR2 | Murine | aaagtttcc | 124 |
| | | sequence | | |
| CAN30G3 | K, CDR3 | Murine | tttcaaggttcacattttccgtggacg | 125 |
| | | sequence | | |
| CAN30G3 | K, CDR1 | Murine | QNIVHSNGNTY | 126 |
| | | sequence | | |
| CAN30G3 | K, CDR2 | Murine | KVS | 127 |
| | | sequence | | |
| CAN30G3 | K, CDR3 | Murine | FQGSHFPWT | 128 |
| | | sequence | | |
| CAN30G3 | K, FR1 | Murine | gatgttttgatgacccaaactccactctccctgcctgtcagtcttgg | 129 |
| | | sequence | agatcaagcctccatctcttgcagatctagt | |
| CAN30G3 | K, FR2 | Murine | ttagaatggtacctgcagaaatcaggccagtctccaaagctcct | 130 |
| | | sequence | gatctac | |
| CAN30G3 | K, FR3 | Murine | aaccgattttctggggtcccagacaggttcagtggcagtggatca | 131 |
| | | sequence | gggacagatttcacactcaagatcagcagagtggaggctgag | |
| | | | gatctgggagtttattactgc | |
| CAN30G3 | K, FR4 | Murine | ttcggtggaggcaccaagctggaaatcaaac | 132 |
| | | sequence | | |
| CAN30G3 | H, | Murine | caggtgcagctgaaggagtcaggacctggcctggtggcgccct | 133 |
| | Variable | sequence | cacagagcctgtccatcacatgcactgtctcagggttctccttaac | |
| | region | | cgactatggtataacctggattcgccagcctccaggaaagggtc | |
| | | | tggagtggctgggagtaatatggggtggtggaagcgcatactat | |
| | | | aatttagttctcaaatccagactgagcatcagcaaggacaactc | |
| | | | caagagtcaagttttcttaaaaatgaacagtctgcaaactgatga | |
| | | | cacagccatgtactactgtgccaaacatcggactttgattacgac | |
| | | | tgctatggactactggggtcaaggaatttcagtcaccgtctcctca | |
| | | | g | |
| CAN30G3 | H, | Murine | QVQLKESGPGLVAPSQSLSITCTVSGFSLTDY | 134 |
| | variable | sequence | GITWIRQPPGKGLEWLGVIWGGGSAYYNLVL | |
| | region | | KSRLSISKDNSKSQVFLKMNSLQTDDTAMYYC | |
| | | | AKHRTLITTAMDYWGQGISVTVSS | |
| CAN30G3 | H, CDR1 | Murine | gggttctccttaaccgactatggt | 135 |
| | | sequence | | |
| CAN30G3 | H, CDR2 | Murine | atatggggtggtggaagcgca | 136 |
| | | sequence | | |
| CAN30G3 | H, CDR3 | Murine | gccaaacatcggactttgattacgactgctatggactac | 137 |
| | | sequence | | |
| CAN30G3 | H, CDR1 | Murine | GFSLTDYG | 138 |
| | | sequence | | |
| CAN30G3 | H, CDR2 | Murine | IWGGGSA | 139 |
| | | sequence | | |
| CAN30G3 | H, CDR3 | Murine | AKHRTLITTAMDY | 140 |
| | | sequence | | |
| CAN30G3 | H, FR1 | Murine | caggtgcagctgaaggagtcaggacctggcctggtggcgccct | 141 |
| | | sequence | cacagagcctgtccatcacatgcactgtctca | |
| CAN30G3 | H, FR2 | Murine | ataacctggattcgccagcctccaggaaagggtctggagtggct | 142 |
| | | sequence | gggagta | |
| CAN30G3 | H, FR3 | Murine | tactataatttagttctcaaatccagactgagcatcagcaaggac | 143 |
| | | sequence | aactccaagagtcaagttttcttaaaaatgaacagtctgcaaact | |
| | | | gatgacacagccatgtactactgt | |
| CAN30G3 | H, FR4 | Murine | tggggtcaaggaatttcagtcaccgtctcctcag | 144 |
| | | sequence | | |
| CAN30G4 | K, | Murine | gatgttttgatgacccaaactccactctccctgcctgtcagtcttgg | 145 |
| | Variable | sequence | agatcaagcctccatctcttgcagatctagtcagaacattgtacat | |
| | region | | agtgatggaaacacctatttagaatggtacctgcagaaaccag | |
| | | | gccagtctccaaagctcctgatctacaaattttccaaccgattttct | |
| | | | ggggtcccagacaggttcagtggcagtggatcagggacagatt | |
| | | | tcacactcaagatcagcagagtggaggctgaggatctgggagt | |
| | | | ttattactgctttcaaggttcacatgttcctcccacgttcggtgctgg | |
| | | | gaccaagctggagctgaaac | |
| CAN30G4 | K, | Murine | DVLMTQTPLSLPVSLGDQASISCRSSQNIVHS | 146 |
| | variable | sequence | DGNTYLEWYLQKPGQSPKLLIYKFSNRFSGVP | |
| | region | | DRFSGSGSGTDFTLKISRVEAEDLGVYYCFQ | |
| | | | GSHVPPTFGAGTKLELK | |
| CAN30G4 | K, CDR1 | Murine | cagaacattgtacatagtgatggaaacacctat | 147 |
| | | sequence | | |
| CAN30G4 | K, CDR2 | Murine | aaattttcc | 148 |
| | | sequence | | |
| CAN30G4 | K, CDR3 | Murine | tttcaaggttcacatgttcctcccacg | 149 |
| | | sequence | | |
| CAN30G4 | K, CDR1 | Murine | QNIVHSDGNTY | 150 |
| | | sequence | | |
| CAN30G4 | K, CDR2 | Murine | KFS | 151 |
| | | sequence | | |
| CAN30G4 | K, CDR3 | Murine | FQGSHVPPT | 152 |
| | | sequence | | |
| CAN30G4 | K, FR1 | Murine | gatgttttgatgacccaaactccactctccctgcctgtcagtcttgg | 153 |
| | | sequence | agatcaagcctccatctcttgcagatctagt | |
| CAN30G4 | K, FR2 | Murine | ttagaatggtacctgcagaaaccaggccagtctccaaagctcct | 154 |
| | | sequence | gatctac | |
| CAN30G4 | K, FR3 | Murine | aaccgattttctggggtcccagacaggttcagtggcagtggatca | 155 |
| | | sequence | gggacagatttcacactcaagatcagcagagtggaggctgag | |
| | | | gatctgggagtttattactgc | |
| CAN30G4 | K, FR4 | Murine | ttcggtgctgggaccaagctggagctgaaac | 156 |
| | | sequence | | |
| CAN30G4 | H, | Murine | gaagtgaagctggtggagtctgggggaggcttagtgaagtctg | 157 |
| | Variable | sequence | gagggtccctgaaactctcctgtgcagtctctggattcactttcagt | |
| | region | | acctatgccatgtcttgggttcgccagattccggagaagaggctg | |
| | | | gagtgggtcgcaaccattagtaatggtggtagttatatctactattc | |
| | | | agacagtgtgaagggtcgattcaccatctccagagacaatgcc | |
| | | | aagaacaccctgtacctgcaaatgagcagtctgaggtctgagg | |
| | | | acacggccatgtattactgtgcacgacatagggagtcctataggt | |
| | | | acgactggtttgcttactggggccaagggactctggtcactgtctc | |
| | | | tgcag | |
| CAN30G4 | H, | Murine | EVKLVESGGGLVKSGGSLKLSCAVSGFTFSTY | 158 |
| | variable | sequence | AMSWVRQIPEKRLEWVATISNGGSYIYYSDSV | |
| | region | | KGRFTISRDNAKNTLYLQMSSLRSEDTAMYYC | |
| | | | ARHRESYRYDWFAYWGQGTLVTVSA | |
| CAN30G4 | H, CDR1 | Murine | ggattcactttcagtacctatgcc | 159 |
| | | sequence | | |
| CAN30G4 | H, CDR2 | Murine | attagtaatggtggtagttatatc | 160 |
| | | sequence | | |
| CAN30G4 | H, CDR3 | Murine | gcacgacatagggagtcctataggtacgactggtttgcttac | 161 |
| | | sequence | | |
| CAN30G4 | H CDR1 | Murine | GFTFSTYA | 162 |
| | | sequence | | |
| CAN30G4 | H CDR2 | Murine | ISNGGSYI | 163 |
| | | sequence | | |
| CAN30G4 | H, CDR3 | Murine | ARHRESYRYDWFAY | 164 |
| | | sequence | | |
| CAN30G4 | H, FR1 | Murine | gaagtgaagctggtggagtctgggggaggcttagtgaagtctg | 165 |
| | | sequence | gagggtccctgaaactctcctgtgcagtctct | |
| CAN30G4 | H, FR2 | Murine | atgtcttgggttcgccagattccggagaagaggctggagtgggt | 166 |
| | | sequence | cgcaacc | |
| CAN30G4 | H, FR3 | Murine | tactattcagacagtgtgaagggtcgattcaccatctccagagac | 167 |
| | | sequence | aatgccaagaacaccctgtacctgcaaatgagcagtctgaggt | |
| | | | ctgaggacacggccatgtattactgt | |
| CAN30G4 | H, FR4 | Murine | tggggccaagggactctggtcactgtctctgcag | 168 |
| | | sequence | | |
| Ravn | GPeΔmu | Synthetic | MKTIYFLISLILIQSIKTLPVLEIASNSQPQDVDS | 169 |
| GPeΔmuc | cΔTM | Marburg | VCSGTLQKTEDVHLMGFTLSGQKVADSPLEA | |
| | | virus | SKRWAFRTGVPPKNVEYTEGEEAKTCYNISV | |
| | | | TDPSGKSLLLDPPSNIRDYPKCKTVHHIQGQN | |
| | | | PHAQGIALHLWGAFFLYDRVASTTMYRGKVF | |
| | | | TEGNIAAMIVNKTVHRMIFSRQGQGYRHMNLT | |
| | | | STNKYWTSSNETQRNDTGCFGILQEYNSTNN | |
| | | | QTCPPSLKPPSLPTVTPSIHSTNTQINTAKSGT | |
| | | | MRPPIYFRKKRSIFWKEGDIFPFLDGLINTEIDF | |
| | | | DPIPNTETIFDESPSFNTSTNEEQHTPPNISLTF | |
| | | | SYFPDKNGDTAYSGENENDCDAELRIWSVQE | |
| | | | DDLAAGLSWIPFFGPGIEGLYTAGLIKNQNNLV | |
| | | | CRLRRLANQTAKSLELLLRVTTEERTFSLINRH | |
| | | | AIDFLLTRWGGTCKVLGPDCCIGIEDLSKNISE | |
| | | | QIDKIRKDEQKEET | |
| Angola | GPeΔmu | Synthetic | MKTTCLLISLILIQGVKTLPILEIASNIQPQNVDS | 170 |
| GPeΔmuc | cΔTM | Marburg | VCSGTLQKTEDVHLMGFTLSGQKVADSPLEA | |
| | | virus | SKRWAFRAGVPPKNVEYTEGEEAKTCYNISV | |
| | | | TDPSGKSLLLDPPTNIRDYPKCKTIHHIQGQNP | |
| | | | HAQGIALHLWGAFFLYDRIASTTMYRGKVFTE | |
| | | | GNIAAMIVNKTVHKMIFSRQGQGYRHMNLTST | |
| | | | NKYWTSSNGTQTNDTGCFGTLQEYNSTKNQ | |
| | | | TCAPSKKPLPLPTAHPEVKLTSTSTDATKLNTT | |
| | | | QHLVYFRRKRNILWREGDMFPFLDGLINAPID | |
| | | | FDPVPNTKTIFDESSSSGASAEEDQHASPNIS | |
| | | | LTLSYFPKVNENTAHSGENENDCDAELRIWSV | |
| | | | QEDDLAAGLSWIPFFGPGIEGLYTAGLIKNQN | |
| | | | NLVCRLRRLANQTAKSLELLLRVTTEERTFSLI | |
| | | | NRHAIDFLLARWGGTCKVLGPDCCIGIEDLSR | |
| | | | NISEQIDQIKKDEQKEGT | |
| Musoke | GPeΔmu | Synthetic | MKTTCFLISLILIQGTKNLPILEIASNNQPQNVD | 171 |
| GPeΔmuc | cΔTM | Marburg | SCSGTLQKTEDVHLMGFTLSGQKVADSPLE | |
| | | virus | ASKRWAFRTGVPPKNVEYTEGEEAKTCYNIS | |
| | | | VTDPSGKSLLLDPPTNIRDYPKCKTIHHIQGQN | |
| | | | PHAQGIALHLWGAFFLYDRIASTTMYRGKVFT | |
| | | | EGNIAAMIVNKTVHKMIFSRQGQGYRHMNLTS | |
| | | | INKYWTSSNGTQINDTGCFGALQEYNSTKN | |
| | | | QTCAPSKIPPPLPTARPEIKLTSTPTDATKLNT | |
| | | | TQHLVYFRRKRSILWREGDMFPFLDGLINAPI | |
| | | | DFDPVPNTKTIFDESSSSGASAEEDQHASPNI | |
| | | | SLTLSYFPNINENTAYSGENENDCDAELRIWS | |
| | | | VQEDDLAAGLSWIPFFGPGIEGLYTAVLIKNQN | |
| | | | NLVCRLRRLANQTAKSLELLLRVTTEERTFSLI | |
| | | | NRHAIDFLLTRWGGTCKVLGPDCCIGIEDLSK | |
| | | | NISEQIDQIKKDEQKEGT | |
| Ci67 | GPeΔmu | Synthetic | MKTTCLFISLILIQGIKTLPILEIASNNQPQNVDS | 172 |
| GPeΔmuc | cΔTM | Marburg | VCSGTLQKTEDVHLMGFTLSGQKVADSPLEA | |
| | | virus | SKRWAFRTGVPPKNVEYTEGEEAKTCYNISV | |
| | | | TDPSGKSLLLDPPTNIRDYPKCKTIHHIQGQNP | |
| | | | HAQGIALHLWGAFFLYDRIASTTMYRGRVFTE | |
| | | | GNIAAMIVNKTVHKMIFSRQGQGYRHMNLTST | |
| | | | NKYWTSNNGTQTNDTGCFGALQEYNSTKNQ | |
| | | | TCAPSKIPSPLPTARPEIKPTSTPTDATTLNTT | |
| | | | QHLVYFRKKRSILWREGDMFPFLDGLINAPIDF | |
| | | | DPVPNTKTIFDESSSSGASAEEDQHASPNISL | |
| | | | TLSYFPNINENTAYSGENENDCDAELRIWSVQ | |
| | | | EDDLAAGLSWIPFFGPGIEGLYTAGLIKNQNNL | |
| | | | VCRLRRLANQTAKSLELLLRVTTEERTFSLINR | |
| | | | HAIDFLLTRWGGTCKVLGPDCCIGIEDLSRNIS | |
| | | | EQIDQIKKDEQKEGT | |
| Ravn | GP | Marburg | MKTIYFLISLILIQSIKTLPVLEIASNSQPQDVDS | 173 |
| (GenBank | | virus, | VCSGTLQKTEDVHLMGFTLSGQKVADSPLEA | |
| Accession | | Kenya | SKRWAFRTGVPPKNVEYTEGEEAKTCYNISV | |
| No. | | 1987 | TDPSGKSLLLDPPSNIRDYPKCKTVHHIQGQN | |
| ACD13005.1) | | | PHAQGIALHLWGAFFLYDRVASTTMYRGKVF | |
| | | | TEGNIAAMIVNKTVHRMIFSRQGQGYRHMNLT | |
| | | | STNKYWTSSNETQRNDTGCFGILQEYNSTNN | |
| | | | QTCPPSLKPPSLPTVTPSIHSTNTQINTAKSGT | |
| | | | MNPSSDDEDLMISGSGSGEQGPHTTLNVVTE | |
| | | | QKQSSTILSTPSLHPSTSQHEQNSTNPSRHAV | |
| | | | TEHNGTDPTTQPATLLNNTNTTPTYNTLKYN | |
| | | | LSTPSPPTRNITNNDTQRELAESEQTNAQLNT | |
| | | | TLDPTENPTTAQDTNSTTNIIMTTSDITSKHPT | |
| | | | NSSPDSSPTTRPPIYFRKKRSIFWKEGDIFPFL | |
| | | | DGLINTEIDFDPIPNTETIFDESPSFNTSTNEEQ | |
| | | | HTPPNISLTFSYFPDKNGDTAYSGENENDCDA | |
| | | | ELRIWSVQEDDLAAGLSWIPFFGPGIEGLYTA | |
| | | | GLIKNQNNLVCRLRRLANQTAKSLELLLRVTTE | |
| | | | ERTFSLINRHAIDFLLTRWGGTCKVLGPDCCIG | |
| | | | IEDLSKNISEQIDKIRKDEQKEETGWGLGGKW | |
| | | | WTSDWGVLTNLGILLLLSIAVLIALSCICRIFTKY | |
| | | | IG | |
| Angola | GP | Marburg | MKTTCLLISLILIQGVKTLPILEIASNIQPQNVDS | 174 |
| (GenBank | | virus, | VCSGTLQKTEDVHLMGFTLSGQKVADSPLEA | |
| Accessoion | | Angola, | SKRWAFRAGVPPKNVEYTEGEEAKTCYNISV | |
| No. | | Lake | TDPSGKSLLLDPPTNIRDYPKCKTIHHIQGQNP | |
| ABE27064.1) | | Victoria, | HAQGIALHLWGAFFLYDRIASTTMYRGKVFTE | |
| | | 2005 | GNIAAMIVNKTVHKMIFSRQGQGYRHMNLTST | |
| | | | NKYWTSSNGTQTNDTGCFGTLQEYNSTKNQ | |
| | | | TCAPSKKPLPLPTAHPEVKLTSTSTDATKLNTT | |
| | | | DPNSDDEDLTTSGSGSGEQEPYTTSDAATKQ | |
| | | | GLSSTMPPTPSPQPSTPQQGGNNTNHSQGV | |
| | | | VTEPGKTNTTAQPSMPPHNTTTISTNNTSKHN | |
| | | | LSTPSVPIQNATNYNTQSTAPENEQTSAPSKT | |
| | | | ILLPTENPTTAKSTNSTKSPITTVPNTINKYST | |
| | | | SPSPTPNSTAQHLVYFRRKRNILWREGDMFP | |
| | | | FLDGLINAPIDFDPVPNTKTIFDESSSSGASAE | |
| | | | EDQHASPNISLTLSYFPKVNENTAHSGENEND | |
| | | | CDAELRIWSVQEDDLAAGLSWIPFFGPGIEGL | |
| | | | YTAGLIKNQNNLVCRLRRLANQTAKSLELLLR | |
| | | | VTTEERTFSLINRHAIDFLLARWGGTCKVLGP | |
| | | | DCCIGIEDLSRNISEQIDQIKKDEQKEGTGWGL | |
| | | | GGKWWTSDWGVLTNLGILLLLSIAVLIALSCIC | |
| | | | RIFTKYIG | |
| Musoke | GP | Marburg | MKTTCFLISLILIQGTKNLPILEIASNNQPQNVD | 175 |
| (GenBank | | virus. | SVCSGTLQKTEDVHLMGFTLSGQKVADSPLE | |
| Accession | | Kenya | ASKRWAFRTGVPPKNVEYTEGEEAKTCYNIS | |
| No. | | 1980 | VTDPSGKSLLLDPPTNIRDYPKCKTIHHIQGQN | |
| CAA78117.1) | | | PHAQGIALHLWGAFFLYDRIASTTMYRGKVFT | |
| | | | EGNIAAMIVNKTVHKMIFSRQGQGYRHMNLTS | |
| | | | INKYWTSSNGTQINDTGCFGALQEYNSTKN | |
| | | | QTCAPSKIPPPLPTARPEIKLTSTPTDATKLNT | |
| | | | TDPSSDDEDLATSGSGSGEREPHTTSDAVTK | |
| | | | QGLSSTMPPTPSPQPSTPQQGGNNTNHSQD | |
| | | | AVTELDKNNTTAQPSMPPHNTTTISTNNTSKH | |
| | | | NFSTLSAPLQNTTNDNTQSTITENEQTSAPSIT | |
| | | | TLPFIGNPTTAKSTSSKKGPATTAPNTTNEHF | |
| | | | TSPPPTPSSTAQHLVYFRRKRSILWREGDMF | |
| | | | PFLDGLINAPIDFDPVPNTKTIFDESSSSGASA | |
| | | | EEDQHASPNISLTLSYFPNINENTAYSGENEN | |
| | | | DCDAELRIWSVQEDDLAAGLSWIPFFGPGIEG | |
| | | | LYTAVLIKNQNNLVCRLRRLANQTAKSLELLLR | |
| | | | VTTEERTFSLINRHAIDFLLTRWGGTCKVLGP | |
| | | | DCCIGIEDLSKNISEQIDQIKKDEQKEGTGWGL | |
| | | | GGKWWTSDWGVLTNLGILLLLSIAVLIALSCIC | |
| | | | RIFTKYIG | |
| Ci67 | GP | Marburg | MKTTCLFISLILIQGIKTLPILEIASNNQPQNVDS | 176 |
| (GenBank | | virus, | VCSGTLQKTEDVHLMGFTLSGQKVADSPLEA | |
| Accession | | Lake | SKRWAFRTGVPPKNVEYTEGEEAKTCYNISV | |
| No. | | Victoria | TDPSGKSLLLDPPTNIRDYPKCKTIHHIQGQNP | |
| ABS17558.1) | | | HAQGIALHLWGAFFLYDRIASTTMYRGRVFTE | |
| | | | GNIAAMIVNKTVHKMIFSRQGQGYRHMNLTST | |
| | | | NKYWTSNNGTQTNDTGCFGALQEYNSTKNQ | |
| | | | TCAPSKIPSPLPTARPEIKPTSTPTDATTLNTT | |
| | | | DPNNDDEDLITSGSGSGEQEPYTTSDAVTKQ | |
| | | | GLSSTMPPTPSPQPSTPQQEGNNTDHSQGT | |
| | | | VTEPNKTNTTAQPSMPPHNTTAISTNNTSKNN | |
| | | | FSTLSVSLQNTTNYDTQSTATENEQTSAPSKT | |
| | | | TLPPTGNLTTAKSTNNTKGPTTTAPNMTNGHL | |
| | | | TSPSPTPNPTTQHLVYFRKKRSILWREGDMFP | |
| | | | FLDGLINAPIDFDPVPNTKTIFDESSSSGASAE | |
| | | | EDQHASPNISLTLSYFPNINENTAYSGENEND | |
| | | | CDAELRIWSVQEDDLAAGLSWIPFFGPGIEGL | |
| | | | YTAGLIKNQNNLVCRLRRLANQTAKSLELLLR | |
| | | | VTTEERTFSLINRHAIDFLLTRWGGTCKVLGP | |
| | | | DCCIGIEDLSRNISEQIDQIKKDEQKEGTGWGL | |
| | | | GGKWWTSDWGVLTNLGILLLLSIAVLIALSCIC | |
| | | | RIFTKYIG | |
| Zaire Ebola | GP | Ebola | MGVTGILQLPRDRFKRTSFFLWVIILFQRTFSIP | 177 |
| virus | | virus, | LGVIHNSTLQVSDVDKLVCRDKLSSTNQLRSV | |
| Mayinga | | 1976 | GLNLEGNGVATDVPSATKRWGFRSGVPPKVV | |
| strain | | | NYEAGEWAENCYNLEIKKPDGSECLPAAPDGI | |
| (GenBank | | | RGFPRCRYVHKVSGTGPCAGDFAFHKEGAFF | |
| Accession | | | LYDRLASTVIYRGTTFAEGVVAFLILPQAKKDF | |
| No. | | | FSSHPLREPVNATEDPSSGYYSTTIRYQATGF | |
| U23187.1) | | | GTNETEYLFEVDNLTYVQLESRFTPQFLLQLN | |
| | | | ETIYTSGKRSNTTGKLIWKVNPEIDTTIGEWAF | |
| | | | WETKKNLTRKIRSEELSFTVVSNGAKNISGQS | |
| | | | PARTSSDPGTNTTTEDHKIMASENSSAMVQV | |
| | | | HSQGREAAVSHLTTLATISTSPQSLTTKPGPD | |
| | | | NSTHNTPVYKLDISEATQVEQHHRRTDNDSTA | |
| | | | SDTPSATTAAGPPKAENTNTSKSTDFLDPATT | |
| | | | TSPQNHSETAGNNNTHHQDTGEESASSGKL | |
| | | | GLITNTIAGVAGLITGGRRTRREAIVNAQPKCN | |
| | | | PNLHYWTTQDEGAAIGLAWIPYFGPAAEGIYIE | |
| | | | GLMHNQDGLICGLRQLANETTQALQLFLRATT | |
| | | | ELRTFSILNRKAIDFLLQRWGGTCHILGPDCCI | |
| | | | EPHWTKNITDKIDQIIHDFVDKTLPDQGDND | |
| | | | NWWTGWRQWIPAGIGVTGVIIAVIALFCICKFV | |
| | | | F | |
| Ebola Zaire | GP | Ebola | MGVTGILQLPRDRFKRTSFFLWVIILFQRTFSIP | 178 |
| 1995, | | virus, | LGVIHNSTLQVSDVDKLVCRDKLSSTNQLRSV | |
| P87666 | | 1995 | GLNLEGNGVATDVPSATKRWGFRSGVPPKVV | |
| | | | NYEAGEWAENCYNLEIKKPDGSECLPAAPDGI | |
| | | | RGFPRCRYVHKVSGTGPCAGDFAFHKEGAFF | |
| | | | LYDRLASTVIYRGTTFAEGVVAFLILPQAKKDF | |
| | | | FSSHPLREPVNATEDPSSGYYSTTIRYQATGF | |
| | | | GTNETEYLFEVDNLTYVQLESRFTPQFLLQLN | |
| | | | ETIYTSGKRSNTTGKLIWKVNPEIDTTIGEWAF | |
| | | | WETKKNLTRKIRSEELSFTAVSNRAKNISGQS | |
| | | | PARTSSDPGTNTTTEDHKIMASENSSAMVQV | |
| | | | HSQGREAAVSHLTTLATISTSPQPPTTKPGPD | |
| | | | NSTHNTPVYKLDISEATQVEQHHRRTDNDSTA | |
| | | | SDTPPATTAAGPLKAENTNTSKGTDLLDPATT | |
| | | | TSPQNHSETAGNNNTHHQDTGEESASSGKL | |
| | | | GLITNTIAGVAGLITGGRRARREAIVNAQPKCN | |
| | | | PNLHYWTTQDEGAAIGLAWIPYFGPAAEGIYT | |
| | | | EGLMHNQDGLICGLRQLANETTQALQLFLRAT | |
| | | | TELRTFSILNRKAIDFLLQRWGGTCHILGPDCC | |
| | | | IEPHDWTKNITDKIDQIIHDFVDKTLPDQGDND | |
| | | | NWWTGWRQWIPAGIGVTGVIIAVIALFCICKFV | |
| | | | F | |
| Ebola | GP | Sudan | MEGLSLLQLPRDKFRKSSFFVWVIILFQKAFS | 179 |
| Sudan Maleo | | ebolavirus, | MPLGVVTNSTLEVTEIDQLVCKDHLASTDQLK | |
| 1979 | | 1979 | SVGLNLEGSGVSTDIPSATKRWGFRSGVPPQ | |
| | | | VVSYEAGEWAENCYNLEIKKPDGSECLPPPP | |
| | | | DGVRGFPRCRYVHKAQGTGPCPGDYAFHKD | |
| | | | GAFFLYDRLASTVIYRGVNFAEGVIAFLILAKPK | |
| | | | ETFLQSPPIREAANYTENTSSYYATSYLEYEIE | |
| | | | NFGAQHSTTLFKINNNTFVLLDRPHTPQFLFQ | |
| | | | LNDTIQLHQQLSNTTGKLIVVTLDANINADIGEW | |
| | | | AFWENKKNLSEQLRGEELSFETLSLNETEDD | |
| | | | DATSSRTTKGRISDRATRKYSDLVPKDSPGM | |
| | | | VSLHVPEGETTLPSQNSTEGRRVDVNTQETIT | |
| | | | ETTATIIGINGNNMQ1STIGTGLSSSQILSSSPT | |
| | | | MAPSPETQTSTTYTPKLPVMTTEEPTTPPRNS | |
| | | | PGSTTEAPTLTTPENITTAVKTVWAQESTSNG | |
| | | | LITSTVTGILGSLGLRKRSRRQVNTRATGKCN | |
| | | | PNLHYWTAQEQHNAAGIAWIPYFGPGAEGIYT | |
| | | | EGLMHNQNALVCGLRQLANETTQALQLFLRA | |
| | | | TTELRTYTILNRKAIDFLLRRWGGTCRILGPDC | |
| | | | CIEPHDWWTKNITDKINQIIHDFIDNPLPNQDNDD | |
| | | | NWWTGWRQWIPAGIGITGIIIAIIALLCVCKLLC | |
| Makona | Virion | Zaire | MGVTGILQLPRDRFKRTSFFLWVIILFQRTFSIP | 180 |
| Ebola virus | spike GP | ebolavirus | LGVIHNSTLQVSDVDKLVCRDKLSSTNQLRSV | |
| (GenBank | precursor | | GLNLEGNGVATDVPSVTKRWGFRSGVPPKVV | |
| Accession | | | NYEAGEWAENCYNLEIKKPDGSECLPAAPDGI | |
| No. | | | RGFPRCRYVHKVSGTGPCAGDFAFHKEGAFF | |
| AJF38895.1 | | | LYDRLASTVIYRGTTFAEGVVAFLILPQAKKDF | |
| | | | FSSHPLREPVNATEDPSSGYYSTTIRYQATGF | |
| | | | GTNETEYLFEVDNLTYVQLESRFTPQFLLQLN | |
| | | | ETIYASGKRSNTTGKLIWKVNPEIDTTIGEWAF | |
| | | | WETKKNLTRKIRSEELSFTAVSNGPKNISGQS | |
| | | | PARTSSDPETNTTNEDHKIMASENSSAMVQV | |
| | | | HSQGRKAAVSHLTTLATISTSPQPPTTKTGPD | |
| | | | NSTHNTPVYKLDISEATQVGQHHRRADNDST | |
| | | | ASDTPPATTAAGPLKAENTNTSKSADSLDLAT | |
| | | | TTSPQNYSETAGNNNTHHQDTGEESASSGKL | |
| | | | GLITNTIAGVAGLITGGRRTRREVIVNAQPKCN | |
| | | | PNLHYWTTQDEGAAIGLAWIPYFGPAAEGIYT | |
| | | | EGLMHNQDGLICGLRQLANETTQALQLFLRAT | |
| | | | TELRTFSILNRKAIDFLLQRWGGTCHILGPDCC | |
| | | | IEPHDVVTKNITDKIDQIIHDFVDKTLPDQGDND | |
| | | | NWWTGWRQWIPAGIGVTGVIIAVIALFCICKFV | |
| | | | F | |
| Zaire Ebola | Surface | Zaire | MGVTGILQLPRDRFKRTSFFLWVIILFQRTFSIP | 181 |
| virus | GP | ebolavirus, | LGVIHNSTLQVSDVDKLVCRDKLSSTNQLRPV | |
| (GenBank | precurso | 1976 | GLNLEGNGVATDVPSATKRWGFRSGVPPKVV | |
| Accession | | | NYEAGEWAENCYNLEIKKPDGSECLPAAPDGI | |
| No. | | | RGFPRCRYVHKVSGTGPCAGDFAFHKEGAFF | |
| AAM76034) | | | LYDRLASTVIYRGTTFAEGVVAFLILPQAKKDF | |
| | | | FSSHPLREPVNATEDPSSGYYSTTIRYQATGF | |
| | | | GTNETEYLFEVDNLTYVQLEPRFTPQFLLQLN | |
| | | | ETIYTSGKRSNTTGKLIWKVNPEIDTTIGEWAF | |
| | | | WETKKNLTRKIRSEELSFTVVSNTHHQDTGEE | |
| | | | SASSGKLGLITNTIAGVAGLITGGRRTRREAIV | |
| | | | NAQPKCNPNLHYVVTTQDEGAAIGLAWIPYFG | |
| | | | PAAEGIYTEGLMHNQDGLICGLRQLANETTQA | |
| | | | LQLFLRATTELRTFSILNRKAIDFLLQRWGGTC | |
| | | | HILGPDCCIEPHDVVTKNITDKIDQIIHDFVDKTL | |
| | | | PD | |
| Makona | Virion | Zaire | MGVTGILQLPRDRFKRTSFFLWVIILFQRTFSIP | 182 |
| Ebola virus | spike GP | ebolavirus | LGVIHNSTLQVSEVDKLVCRDKLSSTNQLRSV | |
| (GenBank | precursor | | GLNLEGNGVATDVPSATKRWGFRSGVPPKVV | |
| Accession | | | NYEAGEWAENCYNLEIKKPDGSECLPAAPDGI | |
| No. | | | RGFPRCRYVHKVSGTGPCAGDFAFHKEGAFF | |
| AAQ55048.1) | | | LYDRLASTVIYRGTTFAEGVVAFLILPQAKKDF | |
| | | | FSSHPLREPVNATEDPSSGYYSTTIRYQATGF | |
| | | | GTNETEYLFEVDNLTYVQLESRFTPQFLLQLN | |
| | | | ETIYTSGKRSNTTGKLIWKVNPEIDTTIGEWAF | |
| | | | WETKKNLTRKIRSEELSFTAVSNTHHQDTGEE | |
| | | | SASSGKLGLITNTIAGVAGLITGGRRARREAIV | |
| | | | NAQPKCNPNLHYVVTTQDEGAAIGLAWIPYFG | |
| | | | PAAEGIYTEGLMHNQDGLICGLRQLANETTQA | |
| | | | LQLFLRATTELRTFSILNRKAIDFLLQRWGGTC | |
| | | | HILGPDCCIEPHDVVTKNITDKIDQIIHDFVDKTL | |
| | | | PD | |
| Sudan | GP | Sudan | MGGLSLLQLPRDKFRKSSFFVWVIILFQKAFS | 183 |
| ebolavirus | | ebolavirus | MPLGVVTNSTLEVTEIDQLVCKDHLASTDQLK | |
| (GenBank | | | SVGLNLEGSGVSTDIPSATKRWGFRSGVPPK | |
| Accession | | | VVSYEAGEWAENCYNLEIKKPDGSECLPPPP | |
| No. | | | DGVRGFPRCRYVHKAQGTGPCPGDYAFHKD | |
| AGL73446) | | | GAFFLYDRLASTVIYRGVNFAEGVIAFLILAKPK | |
| | | | ETFLQSPPIREAVNYTENTSSYYATSYLEYEIE | |
| | | | NFGAQHSTTLFKIDNNTFVRLDRPHTPQFLFQ | |
| | | | LNDTIHLHQQLSNTTGRLIVVTLDANINADIGEW | |
| | | | AFWENKKNLSEQLRGEELSFEALSNITTAVKT | |
| | | | VLPQESTSNGLITSTVTGILGSLGLRKRSRRQT | |
| | | | NTKATGKCNPNLHYVVTAQEQHNAAGIAWIPY | |
| | | | FGPGAEGIYTEGLMHNQNALVCGLRQLANET | |
| | | | TQALQLFLRATTELRTYTILNRKAIDFLLRRWG | |
| | | | GTCRILGPDCCIEPHDVVTKNITDKINQIIHDFID | |
| | | | NPLPN | |

In Table 1, the CDRs are IMGT numbering. H: heavy chain; K: kappa chain.

### Example 3

### Derivation and Screening of Monoclonal Antibodies Binding to Marburg Virus

Monoclonal antibodies (mAbs) to Marburg virus (MARV) were generated by immunizing mice to produce an antibody directed to an epitope in the glycoprotein (GP) of MARV. Immunization of mice was performed according to standard operating procedures.

Six week-old female BALB/c mice (University of Manitoba, using Animal Use Protocols approved by the Protocol Management and Review Committee) were injected subcutaneously (SC) with 20 μg of inert MARV Ravn GPeΔmuc (SEQ ID NO:169) or MARV Angola GPeΔmuc (SEQ ID NO:170) in Freund's Complete Adjuvant (CFA) (Brenntag Biosector) on day 1. On day 32 the mice received 20 μg of the same MARV GP injected intraperitoneally (I.P.) in Incomplete Freund's Adjuvant (IFA) (Brenntag Biosector) in a total volume of 100 μl. On day 56, the mice received 20 μg of the same antigen in a total volume of 100 μl I.P. with IFA. Serum analysis from test bleeds at this point showed specific serum IgG titers to MARV GP (data not shown). Mice received 1-2 boosters of recombinant MARV GP protein (10 μg in IFA I.P.) prior to a final push of 5 μg purified GP (in PBS by IP) before conducting fusions. Standard protocols were used to produce hybridoma cell lines, and monoclonal antibodies were purified on Protein G resin. Mouse sera were screened on Ravn GPeΔmuc, and spleen fusion and hybridoma screening was performed on those mice with specific and strong interaction with Ravn GP. Spleens were harvested three days after the final push and mice were euthanized by anesthesia overdose and exsanguinated by cardiac puncture. The spleens were subsequently excised under aseptic conditions and cell fusions performed essentially according to standard techniques. Positive selected IgG-secreting clones were subjected to large-scale production and subsequent characterization by immunological methods. Isotyping was performed using a commercial murine isotyping dipstick test (Roche) according to the manufacturer's instructions. Hybridoma culture supernatants were concentrated 5-10 fold using Amicon stirred cell nitrogen concentrators with 30 kDa cutoff Millipore (YM-30) membranes (both from Millipore, Billerica, Mass.). Mice immunized with Angola GPeΔmuc purified from S2 cells yielded antibody 40G1. Mice immunized with Ravn GPeΔmuc purified from Gnt−/− HEK293 cells raised antibodies 30G1, 30G3, 30G4 and 30G5. Mice immunized with Ravn GPeΔmuc, and then boosted with a complex of Ravn GPeΔmuc bound to 30G4 Fab (CAN30G4 Fab-Ravn GPeΔmuc), raised antibodies 54G1, 54G2 and 54G3 as presented in Table 2.

TABLE 2
Summary of the MARV and EBOV GP constructs

| Construct | Description* | Deleted Regions* |
|---|---|---|
| MARV GP | Full length GP (1-681) | |
| MARV GPe | Deleted transmembrane | 638-681 (TM) |
| | (TM) domain | |
| MARV GPeΔmuc | Deleted TM & mucin-like | 257-425 (TM) |
| | domain (muc) | 638-681 (muc) |
| MARV GPeΔmucΔw | Deleted TM, muc & GP2 | 257-425 (TM) |
| | wing (w) | 638-681 (muc) |
| | | 436-483 (w) |
| MARV GPcl | Cleaved GP with deleted | 257-425 (TM) |
| | TM & muc | 638-681 (muc) |
| EBOV GP | Full length (1-676) | |
| EBOV GPe | Deleted TM domain | 638-676 |
| EBOV GPeΔmuc-EBOV, | Deleted TM & mucin-like | 314-463 |
| SUDV, BDBV | domain (muc) | |
| EBOV GPeΔmuc- | Deleted TM & mucin-like | 316-470 |
| RESTV | domain (muc) | |

* Numbering of the amino acids begins at the first amino acid of the MARV or EBOV GP signal sequence, as indicated on FIG. 1.

Antibodies were screened via ELISA method against either Ravn GP or Angola GP. Briefly, 96-well MaxiSorp plates (NUNC) were coated with 200 ng/well of antigen, covered and incubated overnight at 4° C. Plates were washed 5× in Milli-Q water to remove any unbound antigen and then blocked with Blocking Buffer (5% Skim Milk Powder (SMP) in Phosphate Buffered Saline (PBS)). Plates were incubated for 1 hour at 37° C. and then washed 5× in Milli-Q water. Plates were then coated with hybridoma supernatant and serially diluted 2-fold in Dilution Buffer (2.5% SMP in PBS) starting at 1 μg/mL. After a 1 hour incubation period at 37° C., plates were then washed 5× in Milli-Q water. Goat anti-Mouse IgG-HRP was then added to the plate at a 1:2000 dilution in Dilution Buffer and incubated again for 1 hour at 37° C. Plates were then washed and substrate added to the plates. Plates were read using 405 nm wavelength after 15 minutes, 30 minutes or 1 hour (incubation at room temperature).

A list of immunogens and boosting immunogens used for selected anti-Marburg GP mAbs is provided in Table 3.

TABLE 3
List of selected anti-Marburg GP antibodies generated

| mAb ID | Immunogen | Boosting Immunogen | Isotype |
|---|---|---|---|
| CAN30G1 | Ravn GPeΔmuc | Ravn GPeΔmuc | IgG1/kappa |
| CAN30G2 | Ravn GPeΔmuc | Ravn GPeΔmuc | IgG1/kappa |
| CAN30G3 | Ravn GPeΔmuc | Ravn GPeΔmuc | IgG1/kappa |
| CAN30G4 | Ravn GPeΔmuc | Ravn GPeΔmuc | IgG1/kappa |
| CAN30G5 | Ravn GPeΔmuc | Ravn GPeΔmuc | IgG1/kappa |
| CAN54G1 | Ravn GPeΔmuc | Ravn GPeΔmuc-CAN30G4 | IgG1/kappa |
| | | Fab complex | |
| CAN54G2 | Ravn GPeΔmuc | Ravn GPeΔmuc-CAN30G4 | IgG1/kappa |
| | | Fab complex | |
| CAN54G3 | Ravn GPeΔmuc | Ravn GPeΔmuc-CAN30G4 | IgG1/kappa |
| | | Fab complex | |
| CAN40G1 | Angola GPeΔmuc | Angola GPeΔmuc | IgG1/kappa |

### Example 4

### ELISA Testing of Mouse Anti-Marburg GP Monoclonal Antibodies

An ELISA was performed to test the binding of the mAbs against multiple strains of Marburg GP, GPe, GPeΔmuc, and to determine if the mAbs are cross-reactive to various strains of Ebola virus (EBOV) GP, GPe and GpeΔmuc. The ELISA plate was coated with 200 ng/well of antigen. The wells were blocked with 5% skim milk then probed with serially diluted generated mAbs starting (0.1 μg/mL to 1 μg/mL). Binding was detected with commercial goat anti-Mouse IgG-HRP. The plate was read at 405 nm after a minimum of 15 minutes incubation with substrate.

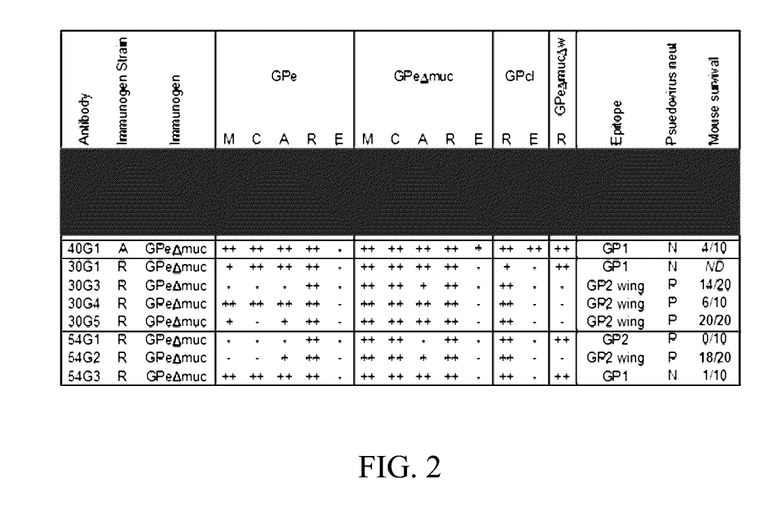

The CAN30, CAN54 and CAN40 series mAbs were all tested for binding to different strains of MARV (Musoke, Ci67, Angola, and Ravn) and EBOV engineered GPs (GPe, GPeΔmuc and GPcl). FIG. 2 lists the results in table form. The results show the binding of all antibodies to MARV GPeΔmuc and GPcl in multiple Marburg strains. CAN30G1, CAN30G4, CAN40G1 and CAN54G3 show binding to the GPe of all MARV strains tested. All other mAbs tested only showed binding to MARV Ravn GPe. CAN30G1, CAN40G1, CAN54G1 and CAN54G3 showed binding to MARV Ravn GPeΔmucΔw. CAN40G1 was the only cross-reactive mAb, showing binding to EBOV GPeΔmuc and EBOV GPcl.

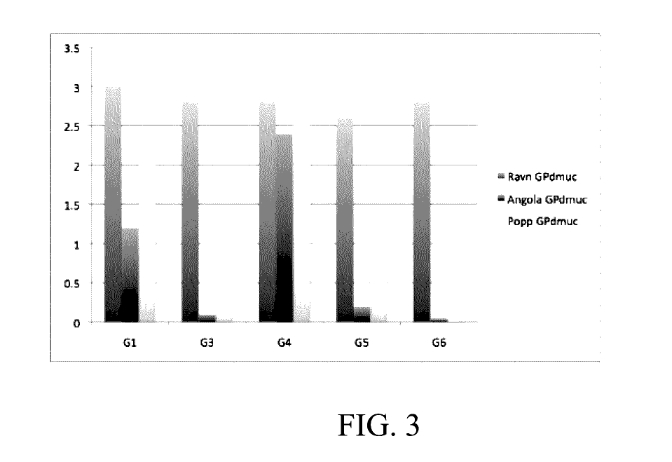

FIG. 3 shows the ELISA results when using the CAN30 series mAbs to test binding to Marburg virus Ravn GPeΔmuc, Angola GPeΔmuc and Popp GPeΔmuc. As shown in FIG. 3, all CAN30 mAbs, except for CAN30G6, showed binding to Ravn GpeΔmuc, Angola GPeΔmuc and Popp GPeΔmuc.

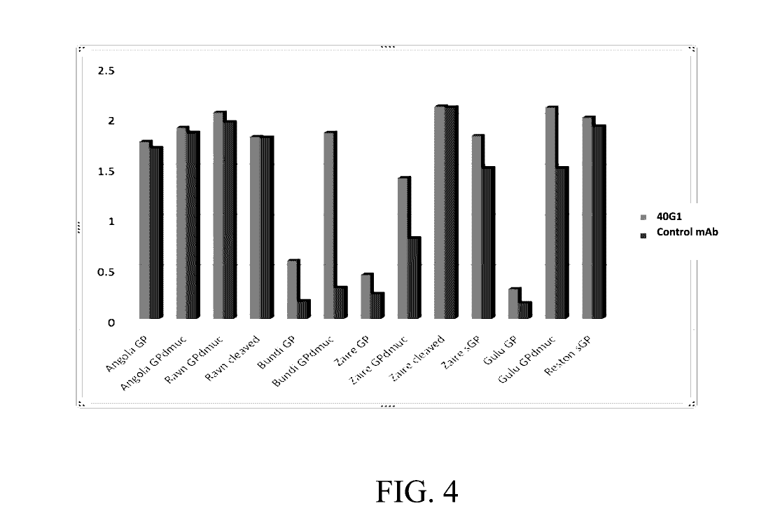

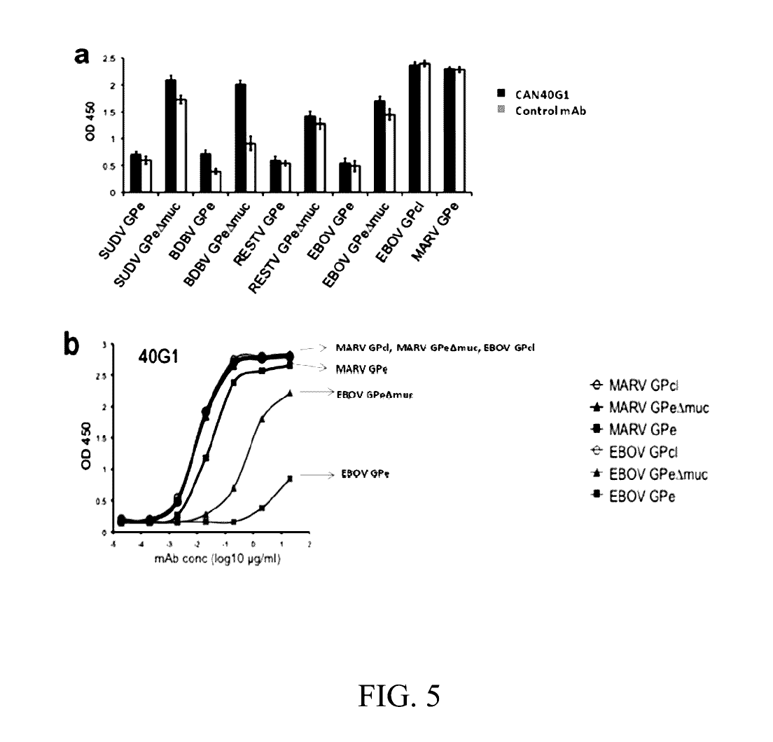

CAN40G1 (anti-MARV Angola mAb) was tested for cross-reactivity to various MARV strains and ebolaviruses. To determine cross-reactivity, ELISAs were performed using the GPeΔmuc of the Ravn, Angola, Popp, and Musoke strains of MARV, the GPeΔmuc of the ebolaviruses EBOV, SUDV, and BDBV, or the cleaved MARV and EBOV GPs (GPcl) as coating antigens. CAN40G1 was further evaluated for binding to the complete, mucin-containing ectodomain of MARV Angola, EBOV, SUDV, and BDBV and to the secreted sGP of EBOV and RESTV. As shown in FIGS. 4, 5A and 5B, CAN40G1 binds to MARV GP and mucin-deleted GP from multiple MARV strains. CAN40G1 is also cross-reactive to EBOV GP as well as EBOV GPeΔmuc.

For animals boosted with CAN30G4 Fab-RavnGPeΔmuc and the CAN54 mAbs that were generated from hybridoma fusions, cross reactivity was directed towards GP1 and GP2 eptitopes that were unique in comparison to CAN40G1 immunized with the Angola strain GPΔmuc. The CAN54 mAbs were also directed away from the immunodominant GP2 wing elicited by immunizations with RavnGPeΔmuc (e.g. CAN30 fusions), however, one mAb clone did result in immunorecognition towards the GP2 wing elicted by several CAN30 mAbs, but specificity against the GPs across the Marburg strains was unique in relation to GPe and GPeΔmuc.

### Example 5

### Western Blots Performed with Mouse Anti-Marburg GP Monoclonal Antibodies

A 4-12% gradient SDS-PAGE is run for 1.5 hours at 200 volts with a combination of MARV and EBOV proteins. The gel is then transferred to a nitrocellulose membrane for a minimum of 1 hour at 45 volts. The membrane is blocked overnight at 4° C. with 5% skim milk in 1×TBST. The next day the mAbs (1° Ab) are diluted in 2.5% skim milk in 1×TBST at concentrations ranging from 2 μg/mL to 5 μg/mL depending on the antibody and used to probe the membrane containing the transferred proteins for 2 hours at room temperature (RT). The membranes are then washed with 1×TBST to remove unbound 1° Ab and probed with anti-mouse IgG-HRP (2° Ab) at a dilution of 1:4000 to 1:5000 for 1.5 hours at RT.

### Example 6

### Pseudovirus Neutralization Assay Performed with Mouse Anti-Marburg GP Monoclonal Antibodies

Antibodies were tested for neutralization of recombinant Vesicular Stomatitis Virus (VSV) pseudotyped with MARV GP. VSV pseudovirions containing a GFP gene in place of the VSV G gene (VSVΔG) and bearing the glycoprotein of MARV Ravn were generated as previously described (Takeda et al. Proc Natl Acad Sci USA, 1997. 94(26): 14764-14769). Experiments were performed in triplicate with VSVΔG bearing either full-length MARV Ravn GP (VSVΔG-GP) or mucin-deleted Δ257-425 GP (VSVΔG-GPΔmuc). Pseudovirions were incubated with anti-VSV G mAb for 1 hour at RT, then incubated with 2.5, 10 or 50 μg/mL of each anti-MARV GP mAb in DMEM-10% FBS for an additional hour. Pseudovirion/mAb complexes were added to Vero cells at a multiplicity of infection (MOI) of 0.01. After 48 hours, infection was evaluated by counting GFP-expressing cells.

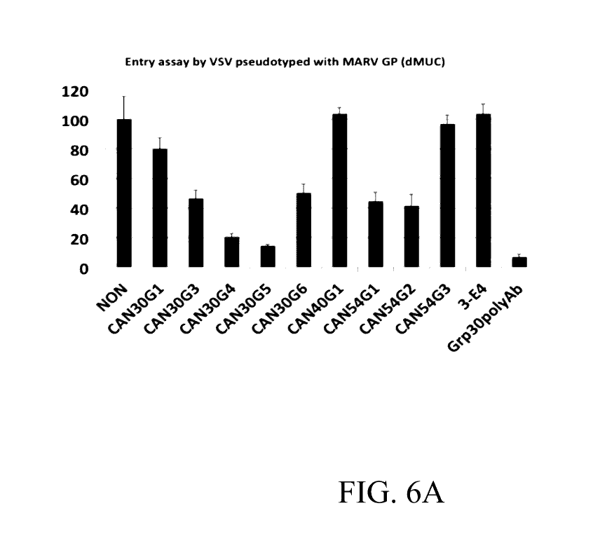

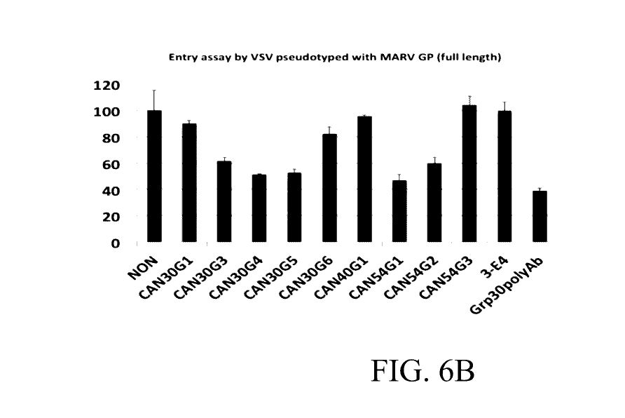

As shown in FIG. 6A, mAbs CAN30G4 and CAN30G5 could suppress infectivity down to 20% or less against mucin-deleted MARV GP. In contrast, polyclonal sera obtained at time of exsanguination from immunized mice (Grp30polyAb) neutralized only slightly better (approximately 10% infectivity remained).

The best neutralizing mAbs in this panel neutralized mucin-containing Marburg virus GP-pseudotypes approximately 50%, while polyclonal sera neutralized the same viruses to approximately 40% infectivity (FIG. 6B). One possible explanation for why the mucin-deleted virus was easier to neutralize than the mucin-containing virus is that the large mucin-like domain could restrict access to antibody epitopes on GP, as has been suggested for Ebola virus. Recent studies in the field suggest that a cocktail of monoclonal antibodies offers better in vivo protection against Ebola virus than individual mAbs alone, and hence, combinations of some of these mAbs may improve upon neutralization capacity over any single mAb alone. mAbs CAN30G3, CAN30G5, CAN54G1 and CAN54G2 neutralized mucin containing (full length) viruses approximately 50% (FIG. 6B). In both examples where antibodies were directed away from the GP2 wing (CAN54G1 and CAN54G3), neutralization was on par with other antibodies directed towards GP1 and GP2 (e.g. CAN30G1 and CAN40G1).

### Example 7

### Epitope Mapping with Pin Peptides

Pin peptides were designed to cover the GP1 and GP2 subunits of Marburg Musoke GP (NCBI Accession number NC_001608) and Marburg Ravn GP (NCBI Accession number AB_04Y1906) by designing 15mers overlapping by 10 amino acids and removing the mucin domain and transmembrane domain along with the signal peptide sequence and cytoplasmic tail (Feldmann et al., 2001, J Gen Virol. 82(Pt 12):2839-2848; Will et al., 1993, J Virol. 67(3):1203-1210). MARV-Angola and MARV-Musoke are approximately 93% identical in GP protein sequence, and MARV and RAVV are approximately 78% identical in GP protein sequence. Because of the similarity between Angola and Musoke, Musoke and Ravn pins were designed. Internal cysteines were replaced by methionine to prevent dimerization of peptide with conserved substitution.

For the assay, pins were activated by rinsing in methanol for a few seconds and allowed to air-dry. Pins were then blocked with 200 μL of Blocking Buffer (1% SMP+1% Tween-20 in PBS) in 96-well round bottom plates and incubated for 2 hours at RT. Pins were then washed with Wash Solution (0.9% w/v NaCl+0.05% Tween-20 in PBS) 3× for ˜1 min/wash. Pins were then immediately coated with 100 μL of a 1/5 dilution of supernatant in Dilution Buffer (0.1% SMP+0.1% Tween-20 in PBS) in new 96-well round bottom plates and left covered overnight at 4° C. The next day, pins were washed 3× in wash solution and then incubated at room temperature for 1 hour in a 1:5000 dilution of Goat anti-mouse IgG-HRP in dilution buffer with 100 μL/well. After incubation, pins were washed 3× in wash solution. ABTS substrate was then applied at 200 μL/well to 96-well flat-bottom MaxiSorp plates and readings taken at 15 minutes, 30 minutes and 1 hour.

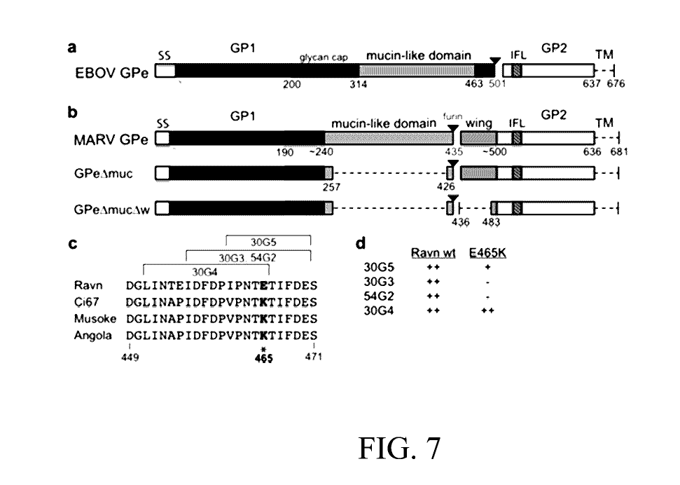

Table 4 shows the results of epitope mapping. FIGS. 7A, 7B, 7C and 7D show the schematics of the MARV GP protein and the epitopes for CAN30G3, CAN30G4, CAN30G5 and CAN54G2. Although the results indicate CAN30G3, CAN30G4, CAN30G5 and CAN54G2 bind to the GP2 subunit of MARV along an overlapping epitope, specificity is modulated more specifically towards the N-terminal residues. These subtle differences are reflected in specificity across the different Marburg strains (FIGS. 2, 6A and 6B). CAN40G1 may bind a conformational epitope. CAN54G1 is shown to bind to GP2 but in a region outside of the N-terminal residues bound by the other GP2 specific antibodies.

TABLE 4
Pin peptide mapping of mAbs to Marburg Ravn GP and Marburg Angola GP

| | Marburg | GP | | Absorbance | |
|---|---|---|---|---|---|
| mAb ID | pins | subunit | Pins | (1 hour) | Peptide Sequence |
| CAN30G1 | Ravn | N/A | N/A | N/A | unreactive to pins |
| | | | | | (tested 2×), likely |
| | | | | | recognizes |
| | | | | | conformational epitope |
| CAN30G2 | Ravn | GP2 | D4 | 0.848 | LINTEIDFDPIPNTE |
| | | | D5 | 0.373 | (SEQ ID NO: 184) |
| | | | | | IDFDPIPNTETIFDE |
| | | | | | (SEQ ID NO: 185) |
| CAN30G3 | Ravn | GP2 | D5 | 1.840 | IDFDPIPNTETIFDE |
| | | | D6 | 1.116 | (SEQ ID NO: 186) |
| | | | | | IPNTETIFDESPSFN |
| | | | | | (SEQ ID NO: 187) |
| CAN30G4 | Ravn | GP2 | D3 | 0.836 | PNLDGLINTEIDFDP |
| | | | D4 | Overflow* | (SEQ ID NO: 188) |
| | | | D5 | 1.966 | LINTEIDFDPIPNTE |
| | | | | | (SEQ ID NO: 189) |
| | | | | | IDFDPIPNTETIFDE |
| | | | | | (SEQ ID NO: 190) |
| CAN30G5 | Ravn | GP2 | D4 | 0.631 | LINTEIDFDPIPNTE |
| | | | D5 | Overflow | (SEQ ID NO: 191) |
| | | | D6 | Overflow | IDFDPIPNTETIFDE |
| | | | | | (SEQ ID NO: 192) |
| | | | | | IPNTETIFDESPSEN |
| | | | | | (SEQ ID NO: 193) |
| CAN30G6 | Ravn | GP1/GP2 | Multiple | N/A | Likely binds |
| | | | | | conformational epitope, |
| | | | | | or repeat sequence |
| CAN54G1 | Ravn | GP1/GP2 | N/A | Multiple | Likely binds |
| | | | | | conformational epitope, |
| | | | | | or repeat sequence |
| CAN54G2 | Ravn | GP2 | D5 | 2.757 | LINTEIDFDPIPNTE |
| | | | D6 | 1.849 | (SEQ ID NO: 194) |
| | | | | | IDFDPIPNTETIFDE |
| | | | | | (SEQ ID NO: 195) |
| CAN54G3 | Ravn | GP2 | D5 | 2.220 | LINTEIDFDPIPNTE |
| | | | D6 | 1.444 | (SEQ ID NO: 196) |
| | | | | | IDFDPIPNTETIFDE |
| | | | | | (SEQ ID NO: 197) |
| CAN40G1 | Ravn | N/A | N/A | N/A | unreactive to pins |
| | | | | | (tested 2×), likely |
| | | | | | recognizes |
| | | | | | conformational epitope |
| CAN40G1 | Musoke | GP1/GP2 | Multiple | N/A | May recognize a repeat |
| | | | | | sequence or |
| | | | | | conformational epitope |

*Note:
Overflow value is greater than 3.9

### Example 8

### Mouse In Vivo Protection Experiments

All procedures with infectious marburgviruses were performed in biosafety level 4 facilities (BSL-4). BALB/c mice were challenged intraperitoneally (IP) with 1000 plaque-forming units (p.f.u.) mouse-adapted MARV. One hour post-exposure, the mice were treated IP with 500 μg (0.5 ml of 1.0 mg/ml mAb in PBS solution) of purified monoclonal antibody or PBS alone. One study also included a negative control group treated with 500 μg of anti-HA IgG at 1.0 mg/mL. Clinical signs for infection were monitored for 28 days post-exposure at which point the study ended, and mice were euthanized.

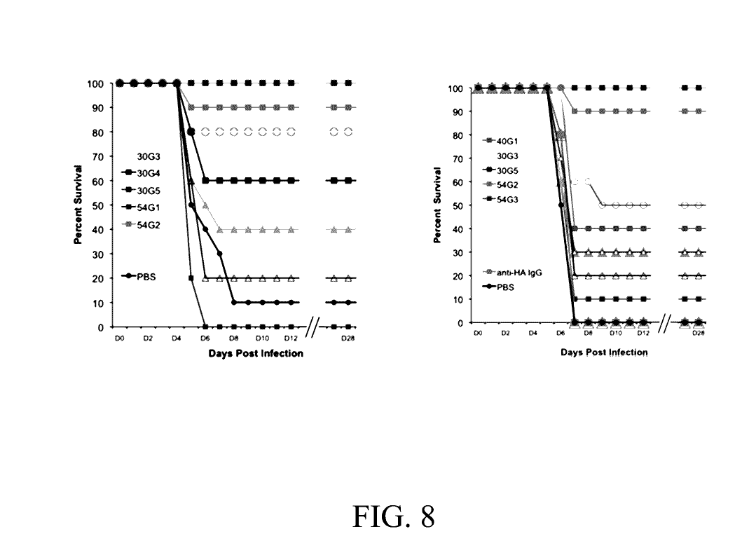

Two separate studies were performed. Table 5 shows the characteristics of the mAbs as well as the results from the in vivo protection studies. FIG. 8 is a line graph showing the results from the two in vivo studies. CAN30G5 and CAN54G2 showed the greatest protection with 100% and 90% survival, respectively, in both studies. CAN30G3 and CAN30G4 showed greater than 50% protection, while CAN40G1 showed 40% protection. In this case, although CAN54G2 and CAN30G3 may be predicted to show equivalent efficacy based on epitope mapping (Example 7) and cross reactivity (FIG. 2) there was a marked difference in vivo against Marburg Ravn challenge in the mouse adapted model.

TABLE 5
Summary of antibodies generated to Marburg Ravn GP, Marburg Angola
GP and Marburg Musoke GP and their protective properties in vivo.

| | | | | Protection in | |
|---|---|---|---|---|---|
| | Cross Reactivity | | VSVdG/GP | BALB/c mice | |
| mAb | with GPeΔmuc | Epitope | Neutralization | Study 1 | Study 2 |
| 30G1 | R, A, M, P | GP1 | 5% | | |
| 30G3 | R, A, M, P | N-term | 40% | 8/10 | 6/10 |
| | | GP2 | | | |
| 30G4 | R, A, M, P | 451-475 | 50% | 6/10 | |
| 30G5 | R, A, M, P | 456-475 | 50% | 10/10 | 10/10 |
| 40G1 | R, A, M, P, Ebov | GP1 | 0% | | 4/10 |
| 54G1 | R, M, P | C-term | 50% | 0/10 | |
| | | GP2 | | | |
| 54G2 | R, A, M, P | N-term | 40% | 9/10 | 9/10 |
| | | GP2 | | | |
| 54G3 | R, A, M, P | GP1 | 0% | 10/10 | 1/10 |
| 61G2 | R, A, M, P | GP1 | 20% | | 1/10 |
| 61G5 | R, A, M, P | unknown | 30% | | |

### Example 9

### V-Gene Sequencing

RNA was isolated from the parental hybridoma clonal cell line using RNeasy Mini Kit. The amplification of V genes from the RNA was performed using the Qiagen OneStep RT-PCR Kit. Several combinations of primer sets were used. The results of the PCR amplification reactions were determined by examining the PCR products on an analytical agarose gel, and the visualized bands at approximately 300-500 bp were gel isolated for cloning. The extracted DNA was directly TA cloned into the pCR2.1-TOPO vector using the low melt agarose method in TOPO TA Cloning manual. The clone reactions were sequenced in both directions using the M13 Forward and M13 Reverse primers. Sequence data was analyzed using DNAStar Lasergene Software.

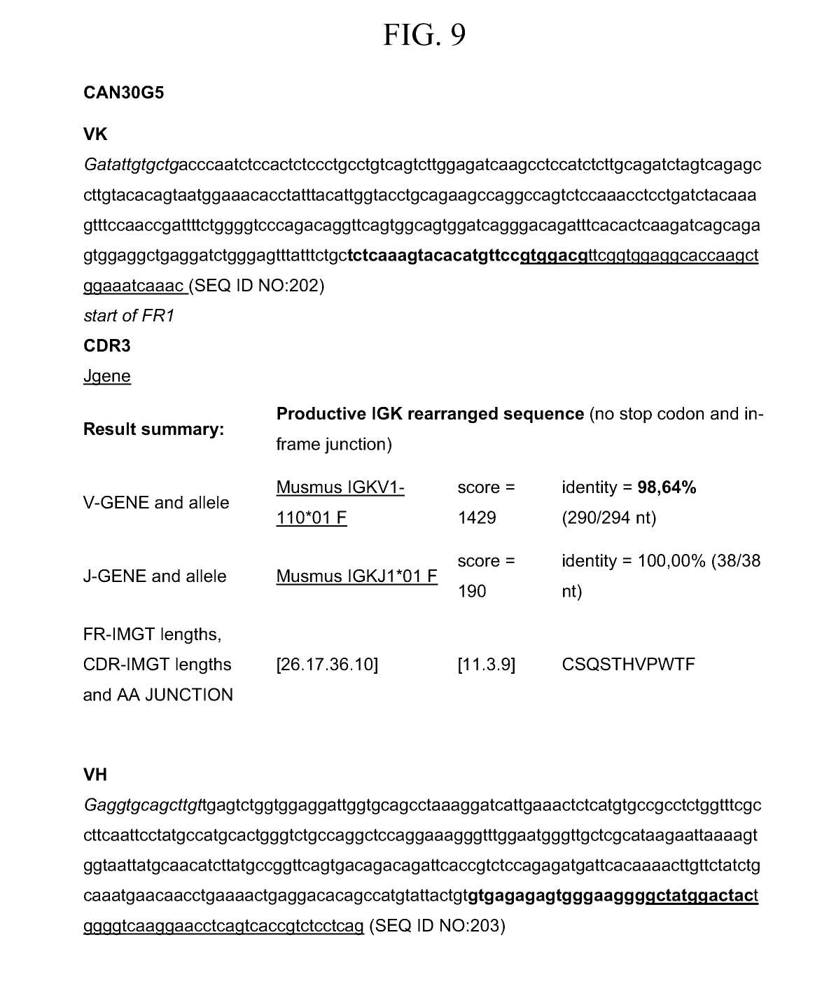

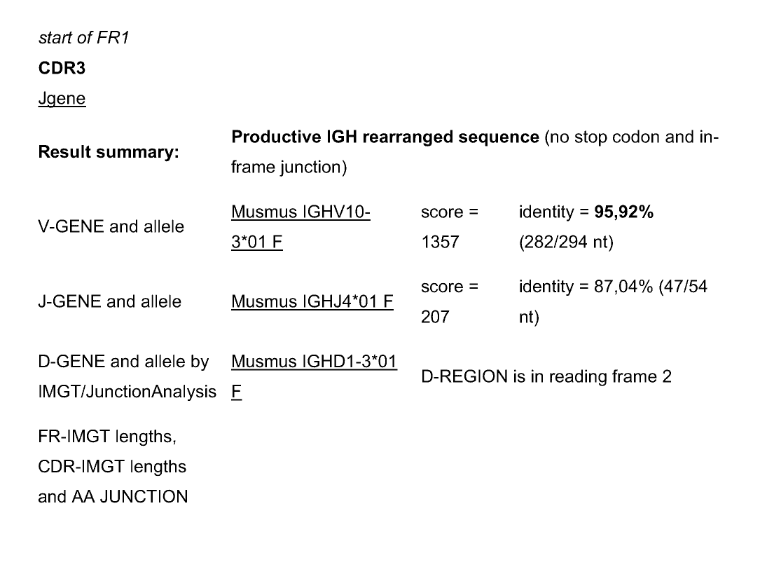

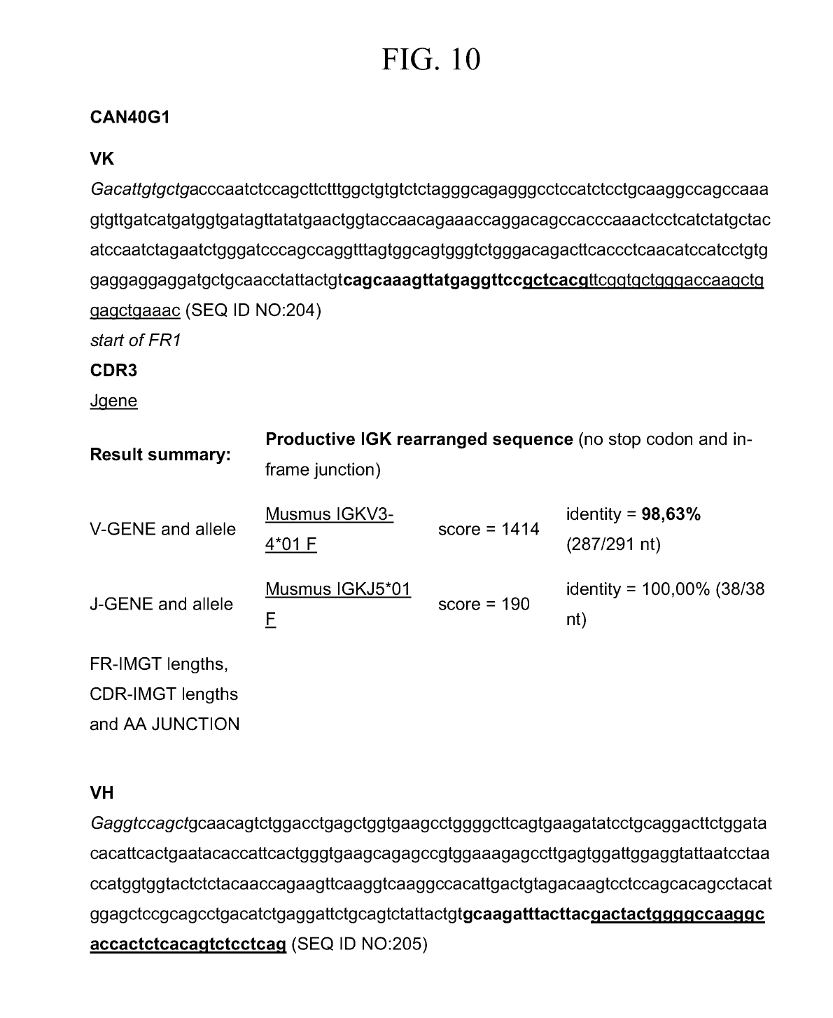

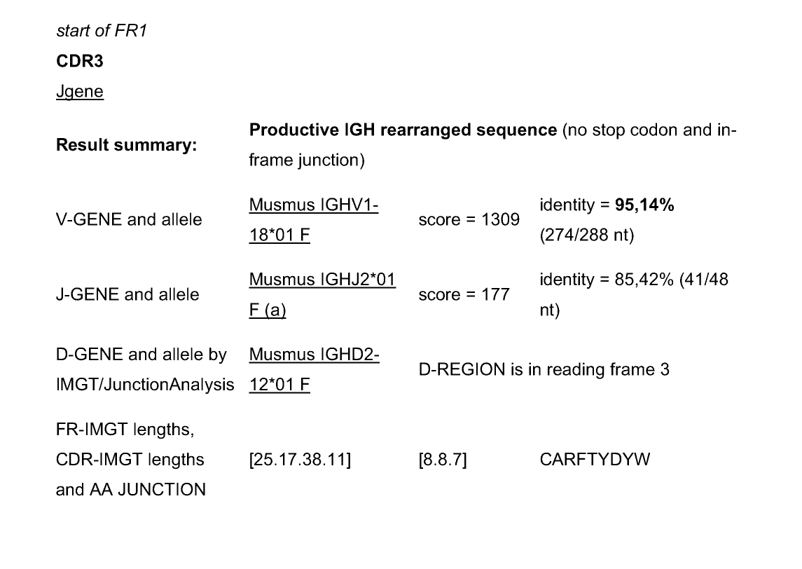

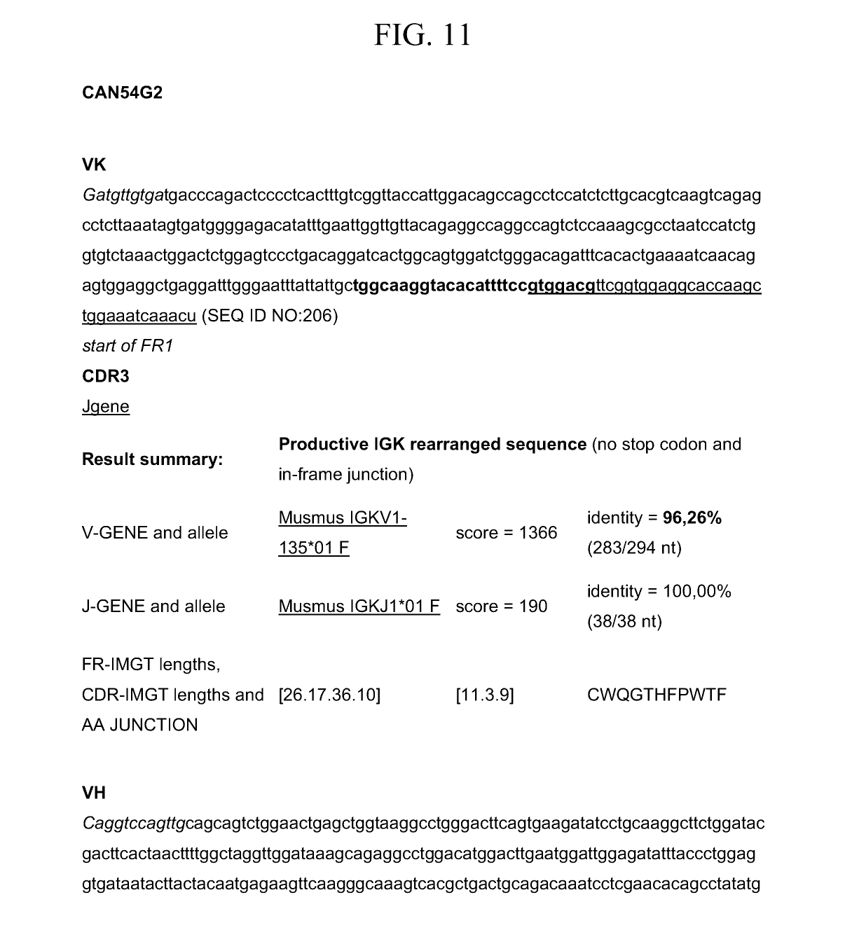

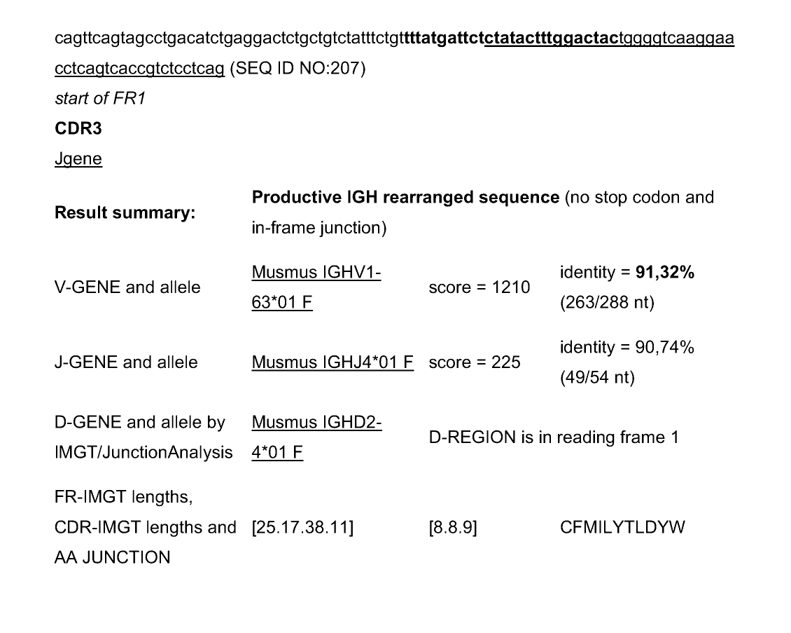

FIGS. 9-11 show the resulting arranged V-gene sequences compared to IMGT/V-Quest reference directory sets and to the NCBI immunoglobulin blast search for CAN30G5, CAN54G2 and CAN40G1. The figures include results for both the VH and VL sequences of the murine parental clone.

### Example 10

### Fab Masking Against Immunodominant Epitope to Modulate Immune Response for Immunotherapy

C. difficile full length or subdomains of Toxins A (TcdA) and B (TcdB) are amplified from Clostridium difficile strain ATCC43255 genomic DNA and ligated into pHis1522 shuttle expression vector with a C terminal poly-His tag (6×His) to facilitate purification. The vectors are then transformed into Bacillus megaterium protoplasts (Mo Bi Tec system, Goettingen, Germany) which are designed for protein expression (Yang G et al, 2008, BMC Microbiology, 8(1):192; Burger S et al, 2003, BBRC, 307(3):584-588). The toxins A and B are expressed in the cells with D-xylose induction and harvested by lysing the cells using a dry ice/ethanol bath. The supernatant is purified on a Ni²⁺ column, eluted by chelation and buffer-exchanged into PBS. Protein concentrations of purified antigen(s) were determined using Pierce BCA assay (Fisher Scientific, Ottawa, Canada).

Purified mAbs for blocking immunodominant epitopes on C. difficile Toxin A Fragment 4, Toxin B Fragment 1 or Toxin B Fragment 4 are derived from hybridoma cell culture (murine variant) or mammalian cell culture (human variant) as described in WO 2013/028810 and WO 2014/085749. Purified mAbs are treated with papain to generate Fab or pepsin to creae F(ab′)₂ and incubated in a molar excess with recombinant TcdA, TcdB or subdomains as described in Example 1. The immunogenic composition (complexed mAb Fab or F(ab′)₂: Toxin) can then be used as an immunogen and/or booster during immunization of mice as described in Example 2 and antisera screened for high titres against the non-immunodominant epitopes. The immunosera of mice immunized and boosted with TcdA:CAN20G2Fab immunogenic complex, is screened for high titers against recombinant TcdAΔF4 to identify mAbs against fragments 1, 2 or 3 of TcdA. Likewise mice immunized and boosted with TcdB:CAN46G4Fab:CAN46G13aFab immunogenic complex, undergoing screening of immunosera for high titers against recombinant TcdBΔF1ΔF4 for the creation of mAbs against fragments 2 and 3 of TcdB. Employing different combinations of immunogenic complexes and screening for the desired immune response allows the identification of immunized mice for the creation of hybridoma cell lines with mAbs directed against non-immunodominant epitopes.

### Example 11

### Modulating Hyperimmune for Passive Immunization

Antibodies play a major role in protective immunity by neutralizing toxins from numerous pathogens and plants. While the exact mechanism of protection is not always fully understood, many vaccines and passive antibody therapies are based on this fact. For ricin, toxin neutralization is believed to involve multiple mechanisms and efforts are often directed towards the A-chain outside the cell or prevention of attachment by raising anti-B chain mAbs. In addition, treatment options for ricin and other toxins often employ xenogeneic antibodies (e.g. IgGs recovered from horses immunized with antigen) for use as a passive immunotherapy often called hyperimmune. In these instances in order to avoid serum sickness, the antibodies are prepared in the form of Fab/F(ab′)₂, and therefore rely on the antibody actions related to the variable region (neutralization) at the loss of the Fc region (effector functionality). For a hyperimmune, the potency is related to the concentration of effective mAbs in the immunoglobulin fraction.

In the current example one would block immunodominant epitopes that are known to be non-neutralizing or non-protective against an antigen used for the production of hyperimmunes to direct the immune response against the known effective epitopes. In this manner the dilution of neutralizing/protective antibodies by high titres against ineffective epitopes can be directed those known to be neutralizing or protective.

The scope of the present inventions is not limited by what has been specifically shown and described herein above. Those skilled in the art will recognize that there are suitable alternatives to the depicted examples of materials, configurations, constructions and dimensions. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

1. A method of inducing an immune response in a subject comprising:
(i) administering to the subject an immunogenic composition at least one time wherein the immunogenic composition masks at least one immunodominant epitope on an immunogenic antigen, and
(ii) inducing an immune response in the subject to at least one non-immunodominant epitope on the immunogenic antigen.

2. The method of claim 1, further comprising administering the immunogenic antigen to a subject at least one time.

3. The method of claim 1 or 2, wherein the immunogenic antigen comprises at least one immunodominant epitope and at least one non-immunodominant epitope.

4. The method of any one of claims 1-3, wherein the immunogenic composition comprises an antigen-binding protein complex.

5. The method of claim 4, wherein the binding protein is an antibody or an antigen-binding portion thereof.

6. The method of claim 4 or 5, wherein the binding protein binds to an immunodominant epitope on the immunogenic antigen.

7. The method of claim 5 or 6, wherein the antibody or antigen-binding portion is selected from the group consisting of: (a) a whole immunoglobulin; (b) an scFv; (c) a Fab fragment; (d) an F(ab′)₂; and (e) a disulfide linked Fv.

8. The method of any one of the preceding claims, wherein the immunogenic antigen is an infectious organism antigen or a tumor cell antigen.

9. The method of claim 8, wherein the infectious organism antigen is a viral antigen or a bacterial antigen.

10. The method of claim 9, wherein the viral antigen is a filovirus antigen.

11. The method of claim 10, wherein the filovirus antigen is a Marburg virus antigen or an Ebola virus antigen.

12. The method of claim 10 or 11, wherein the filovirus antigen is a filovirus glycoprotein.

13. The method of claim 12, wherein the filovirus glycoprotein comprises the GP2 subunit or the GP1 subunit of the Marburg virus glycoprotein.

14. The method of claim 9, wherein the bacterial antigen is a Clostridium difficile antigen.

15. The method of claim 14, wherein the Clostridium difficile antigen is C. difficile toxin A or C. difficile toxin B.

16. The method of any one of the preceding claims, wherein the immune response is a B-cell response.

17. The method of any one of the preceding claims, wherein the immune response is the production of an antibody specific to a non-immunodominant epitope on the immunogenic antigen.

18. The method of claim 17, further comprising harvesting the antibody specific to a non-immunodominant epitope on the immunogenic antigen from the subject.

19. The method of claim 17 or 18, wherein the antibody specific to a non-immunodominant epitope on the immunogenic antigen is a neutralizing antibody.

20. The method of any one of the preceding claims, wherein the subject is non-human.

21. Use of an immunogenic composition that masks at least one immunodominant epitope on an immunogenic antigen for inducing an immune response to at least one non-immunodominant epitope on the immunogenic antigen in a subject.

22. An antigen-binding protein complex comprising at least one binding protein bound to at least one immunodominant epitope on an immunogenic antigen.

23. The antigen-binding protein complex of claim 22, wherein the binding protein is an antibody or an antigen-binding portion thereof.

24. The antigen-binding protein complex of claim 23, wherein the antibody or antigen-binding portion is selected from the group consisting of: (a) a whole immunoglobulin; (b) an scFv; (c) a Fab fragment; (d) an F(ab′)₂; and (e) a disulfide linked Fv.

25. A neutralizing antibody that binds specifically to a non-immunodominant epitope on an immunogenic antigen, wherein the antibody is produced by the method of any one of claims 17-20.
